(19) United States
(12) Patent Application Publication
(10) Pub. No.: US 2023/0201319 A1
(43) Pub. Date: Jun. 29, 2023

(54) ADAMTS13 COMPOSITIONS AND METHODS FOR TREATING AND DIAGNOSING COMPLICATIONS OF CORONAVIRUS DISEASE

(71) Applicant: TAKEDA PHARMACEUTICAL COMPANY LIMITED, Osaka (JP)

(72) Inventors: Peter TURECEK, Klosterneuburg (AT); Bjorn MELLGARD, Cambridge, MA (US); Bruce EWENSTEIN, Brookline, MA (US); Nisha JAIN, North Potomac, MD (US); Wolfhard Werner ERDLENBRUCH, Feldafing (DE)

(73) Assignee: TAKEDA PHARMACEUTICAL COMPANY LIMITED, Osaka (JP)

(21) Appl. No.: 17/999,485

(22) PCT Filed: May 21, 2021
(86) PCT No.: PCT/IB2021/000348

§ 371 Date: Nov. 21, 2022

Related U.S. Application Data
(60) Provisional application No. 63/029,144, filed on May 22, 2020.
(60) Provisional application No. 63/050,645, filed on Jul. 10, 2020.
(60) Provisional application No. 63/078,555, filed on Sep. 15, 2020.

(51) Int. Cl. A61K 38/48; G01N 33/86

(52) U.S. Cl. CPC A61K 38/4886; G01N 33/86; C12Y 304/24087; G01N 2333/755; G01N 2333/96486; G01N 2800/226; G01N 2800/50

(57) ABSTRACT

Provided herein are methods and compositions for treating a coronavirus disease, and particularly its complications, in a subject infected with a pathogenic coronavirus, such as a subject infected with a SARS-CoV-2 or suffering from one or more signs or symptoms of COVID-19. The composition comprises a therapeutically effective amount of an isolated or recombinant disintegrin and metalloproteinase with a thrombospondin type 1 motif (ADAMTS13) protein. The present disclosure further relates to methods for diagnosing a coagulopathy in subject infected with a coronavirus disease, particularly SARS-CoV-2, or suffering from one or more signs or symptoms of COVID-19. The method comprises testing for elevated levels of VWF, depressed levels of ADAMTS13, and the presence of UHMW VWF multimers. These factors, in combination, indicate the presence of a coagulopathy.

### CROSS REFERENCE TO RELATED APPLICATIONS

This patent application claims priority to U.S. Provisional Application No. 63/078,555, filed Sep. 15, 2020, U.S. Provisional Application No. 63/050,645, filed Jul. 10, 2020, and U.S. Provisional Application No. 63/029,144, filed May 22, 2020, the disclosure of each of which is herein incorporated by reference in its entirety.

### TECHNICAL FIELD

Provided herein are methods and compositions for treating and/or diagnosing a coronavirus disease or a condition associated with a coronavirus disease, and particularly its complications, in a subject infected with a pathogenic coronavirus, such as a subject infected with a SARS-CoV-2 or suffering from one or more signs or symptoms of COVID-19. The composition comprises a therapeutically effective amount of an isolated or recombinant A Disintegrin-like And Metalloprotease with Thrombospondin type I motif No. 13 (ADAMTS13) protein. Methods of treatment comprise administering to the subject a therapeutically effective amount of an isolated or recombinant ADAMTS13 at times and dosages sufficient to prevent or reduce one or more conditions, symptoms, risks, or complications of a coronavirus infection or disease, e.g. SARS-CoV-2 or COVID-19. These conditions include, for example and not limited to, elevated levels of von Willebrand factor (VWF) and/or its multimers (especially ultralarge multimers), reduced levels of endogenous ADAMTS13, elevated cytokine levels, coagulopathies, blood-clotting disorders, veno-occlusive disorders, prothrombotic conditions, ARDS, COPD, pneumonia, asthma, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke). Diagnostic methods comprise evaluating a subject or patients for elevated levels of VWF and/or its multimers (especially ultralarge multimers), reduced levels of endogenous ADAMTS13, or combinations thereof.

Administration of ADAMTS13 will advantageously treat, protect, rescue, aid, or maintain recovery of a coronavirus subject, particularly a COVID-19 subject, from adverse consequences of infection, particularly SARS-CoV-2 infection, and particularly in subjects presenting excessive ultralarge VWF multimers, excessive cytokine levels, blood disorders involving undesirable coagulation, infarction, thrombosis, or embolism, or other risk factors.

### BACKGROUND OF THE INVENTION

COVID-19 is an infectious disease caused by severe acute respiratory syndrome coronavirus 2 (SARS-CoV-2). Subjects infected with this virus may or may not develop symptoms. When present, the symptoms of COVID-19 may range from mild to very severe, and the disease is fatal in a substantial portion and number of subjects. The World Health Organization declared COVID-19 a worldwide pandemic, with millions of cases and many thousands of deaths across 187 countries and territories. Common symptoms of COVID-19 include fever, cough, fatigue, shortness of breath, and loss of smell and taste. Symptoms may progress and become life-threatening, including viral pneumonia, cytokine storm, and multi-organ failure. More than 50% of severe COVID-19 cases and almost all fatal cases have coagulopathies (bleeding disorders). Cases have also been described that indicate a clotting disorder, including reports that thrombosis (DVT) and pulmonary embolism (PE) can be a significant complication, particularly in late stages of the disease. Patients developed life-threatening complications, and significant numbers of patients died, despite administration of a prophylactic anticoagulant (heparin). See, Lodigiani et al., Venous and arterial thromboembolic complications in COVID-19 patients admitted to an academic hospital in Milan, Italy, Thromb. Res. 2020 Apr. 23; 191:9-14).

Acute pulmonary embolism (PE) or deep vein thrombosis (DVT) has been reported in cases of COVID-19 pneumonia, including patients with onset of PE after a cytokine storm, and despite DVT prophylaxis (enoxaparin). See, Griffin, et al., Pulmonary Embolism and Increased Levels of d-Dimer in Patients with Coronavirus Disease, Emerg. Infect. Dis. 2020 Apr. 29; 26(8). While the pathophysiology of thrombosis in COVID-19 is unknown, excessively high VWF and FVIII levels have been reported in infected patients. Administration of particularly high doses of one or more of dalteparin and heparin has been described. See, Escher et al., Severe COVID-19 infection associated with endothelial activation, Thromb. Res. 2020 Apr. 15; 190:62 (Escher 190:62). A possible explanation for such high levels of VWF and FVIII is that SARS-CoV-2 directly infects endothelial cells, resulting in endotheliitis. See, Varga, et al., Lancet. 2020 Apr. 20. pii: S0140-6736(20)30937-5. Theoretically, infected cells would release proteins from their storage granules, such as Weibel-Palade bodies, which contain and would release high and ultralarge forms of VWF. In one report, a COVID-19 patient also suffering from immune thrombocytopenic purpura (TTP) was studied, although the investigators did not look for and did not measure any involvement of VWF. See, Zulfiqar, et al., Immune Thrombocytopenic Purpura in a Patient with Covid-19, N. Engl. J. Med. 2020 Apr. 30; 382(18):e43.

Increased levels of VWF may also result from elevated levels of inflammatory cytokines, such as IL-8, tumor necrosis factor alpha (TNF-α), and IL-6, which are thought to be significant components of the acute phase of COVID-19 infection. These cytokines have been demonstrated to induce the release of ultralarge molecular weight forms of VWF (i.e., multimers) and to inhibit VWF cleavage by ADAMTS13. See, Bernado et al., Effects of inflammatory cytokines on the release and cleavage of the endothelial cell-derived ultralarge von Willebrand factor multimers under flow, Blood 2004 Jul. 1; 104(1):100-6.

Excessive VWF in COVID-19 subjects, particularly in ultralarge forms, may contribute to or be the main cause of infarction (anemic or hemorrhagic) leading to thrombosis or embolism, when it occurs. An infarction is the process resulting in a macroscopic area of necrotic tissue in an organ caused by loss of adequate blood supply; it can be serious, sometimes fatal, and is irreversible. According to the invention, administration of an effective amount of ADAMTS13 would reduce or prevent excessive VWF, particularly in its ultralarge form, and would serve as a treatment for COVID-19 related clotting disorders, infarction, thrombosis, embolism, and related disorders and complications.

COVID-19 subjects may be particularly susceptible or subject to a number of conditions or complications, such as elevated levels of VWF and/or its multimers (especially ultralarge multimers), reduced levels of endogenous ADAMTS13, elevated cytokine levels, coagulopathies, blood-clotting disorders, veno-occlusive disorders, prothrombotic conditions, ARDS, COPD, pneumonia, asthma, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof (collectively, “risk factors” or “complications”). Any one or more of these may contribute the severity of COVID-19 and may be life-threatening.

Early experience from the COVID-19 pandemic has shown that in susceptible individuals SARS-CoV-2 infection results in a thrombotic coagulopathy that is a frequent cause of adverse clinical outcomes. See, Tang N, Li D, Wang X, Sun Z. Abnormal coagulation parameters are associated with poor prognosis in patients with novel coronavirus pneumonia. J Thromb Haemost 2020; 18:844-7; Goshua G, Pine A B, Meizlish M L, et al. Endotheliopathy in COVID-19-associated coagulopathy: evidence from a single-centre, cross-sectional study. Lancet Haematol. 2020; 7(8): e575-e582. It is proposed that coagulopathy is primarily the consequence of vascular endothelial dysfunction driven by an exaggerated host inflammatory response to SARS-CoV-2 or from the direct effect of viral replication within endothelial cells. See, G. Goshu et al., supra; Varga, A. J. et al., Lancet 395 (10234) (2020) 1417-1418. In severe COVID-19, this results in the deposition of platelet and fibrin rich thrombi in small pulmonary blood vessels and sometimes thrombosis in large peripheral veins or arteries. See, Wichmann D et al., Ann Intern Med 2020; Cui S et al., J Thromb Haemost 2020; 18:1421-4; Varatharaj A, Thomas N, Ellul M A, et al. Neurological and neuropsychiatric complications of COVID-19 in 153 patients: a UK-wide surveillance study. Lancet Psychiatry 2020.

In line with this model of pathogenesis, plasma markers of endothelial activation are consistently elevated in severe COVID-19 and correlate with adverse outcomes (Gosha, supra). These include von Willebrand factor (VWF) which is a critical mediator of adhesive interactions between circulating platelets and the damaged vessel wall in the normal hemostatic response. See, South K et al., J Thromb Haemost 2018; 16:6-18. VWF is synthesized, stored in Weibel-Palade bodies and released by endothelial cells as biologically highly active ultra-high molecular weight (UHMW) multimers. These UHMW are highly effective mediators of platelets adhesion to other platelets and to subendothelial structures exposed upon vessel damage. However, under normal circumstances UHMW VWF multimers are cleaved to less adhesive small multimers with lower thrombogenic potential by ADAMTS13 (a disintegrin and metalloprotease with a thrombospondin type 1 motif member 13) See, Dong J F et al., Blood 2002; 100:4033-9. Disorders such as thrombotic thrombocytopenic purpura (TTP) in which there is a severe autoimmune-mediated or hereditary deficiency of ADAMTS13 activity result in the persistence of circulating UHMW VWF multimers. This manifests as the formation of abnormal platelet rich thrombi in small arterioles that frequently leads to life-threatening microvascular occlusive crises and ischemic tissue injury. See, Scully M, Yarranton H, Liesner R, et al. Regional UK TTP registry: correlation with laboratory ADAMTS 13 analysis and clinical features. Br J Haematol 2008; 142:819-26.

Since there are some similarities with the micro-occlusive vasculopathy of disorders such as TTP and the manifestations of SAR-CoV-2 infection, it has been proposed that abnormal interactions between platelets and endothelium mediated by VWF may also contribute to thrombosis in severe COVID-19. See, Morici N et al., Haemost 2020; O'Sullivan J M, Gonagle D M, Ward S E, Preston R J S, O'Donnell J S. Endothelial cells orchestrate COVID-19 coagulopathy. Lancet Haematol 2020, 7(8):e553-e555; Escher R, Breakey N, Lammle B. ADAMTS13 activity, von Willebrand factor, factor VIII and D-dimers in COVID-19 inpatients. Thromb Res 2020; 192:174-5 (Escher 192:174). Recent case reports and investigations in small patient cohorts substantiate that severe COVID-19 is associated with a marked elevation in circulating VWF levels, in most reports accompanied by reduced ADAMTS13 See, Gosher, supra.; Blasi A, von Meijenfeldt F A, Adelmeijer J, et al. In vitro hypercoagulability and ongoing in vivo activation of coagulation and fibrinolysis in COVID-19 patients on anticoagulation [published online ahead of print, 2020 Aug. 6]. J Thromb Haemost. 2020; 10.1111/jth.15043. doi:10.1111/jth.15043; Martinelli N, Montagnana M, Pizzolo F, et al. A relative ADAMTS13 deficiency supports the presence of a secondary microangiopathy in COVID 19 [published online ahead of print, 2020 Jul. 18]. Thromb Res. 2020; 193:170-172. doi:10.1016/j.thromres.2020.07.034; Makatsariya A D, Slukhanchuk E V, Bitsadze V O, et al. Thrombotic microangiopathy, DIC-syndrome and COVID-19: link with pregnancy prothrombotic state [published online ahead of print, 2020 Jul. 6]. J Matem Fetal Neonatal Med. 2020; 1-9. doi:10.1080/14767058.2020.1786811). Potential mechanisms through which SARS-CoV-2 infection causes endothelial cell activation and associated intravascular coagulopathy in COVID-19 have been hypothesized but are not yet finally explored. See, Goshua, supra; O'Sullivan, supra; Huertas A et al., Eur Respir J. 2020; 56(1):2001634).

Given the severity of SARS-CoV-2 infection and COVID-19, the life-threatening risk of complications, especially blood clotting, infarction, thrombosis, or embolism, and particularly DVT or PE; and given the lack of therapeutic options; there is a pressing need for treatment (including prophylaxis) in affected subjects. According to the invention, administration of an effective amount of isolated or recombinant ADAMTS13 will be a viable treatment option.

### BRIEF SUMMARY OF THE INVENTION

Provided herein are methods and compositions for treating and/or diagnosing a coronavirus disease or a condition associated with a coronavirus disease, and particularly its complications, in a subject infected with a pathogenic coronavirus, such as a subject infected with a SARS-CoV-2 or suffering from one or more signs or symptoms of COVID-19. The invention provides compositions and methods for treating coronavirus associated diseases (e.g., COVID-19) the their related blood disorders, such as coagulopathies (e.g., thrombotic coagulopathies) or clotting disorders; elevated levels of VWF, such as ultra-high molecular weight (UHMW) VWF multimers; thrombosis, such as DVT; embolism, such as pulmonary embolism and renal embolisms resulting in kidney failure. The composition comprises a therapeutically effective amount of isolated or recombinant ADAMTS13. The method comprises administering to a subject infected with a coronavirus (e.g., SARS-CoV-2), or diagnosed with and/or exhibiting a coronavirus associated disease (e.g., COVID-19), a composition comprising a therapeutically effective amount of an isolated or recombinant ADAMTS13. In certain embodiments, the ADAMTS13 is administered at particular dosages and times after detection of the coronavirus (e.g., SARS-CoV-2) infection or diagnosis of a coronavirus associated disease (e.g., COVID-19). Dosage and timing of administration depends upon one or more factors, including for example, the severity or progression of the coronavirus associated disease (e.g., COVID-19) at the time of diagnosis or ADAMTS13 treatment, the subject's age, the subject's sex, the subject's pre-existing conditions, and the subject's predisposition for, risk of, or vulnerability to one or more symptoms, complications, or risk factors associated with the coronavirus associated disease (e.g., COVID-19).

In certain aspects, the invention provides a method of treating or preventing at least one condition or complication in a subject infected with SARS-CoV-2 or suffering from COVID-19, comprising administering a composition comprising isolated or recombinant ADAMTS13. In certain embodiments, the subject is administered the composition comprising isolated or recombinant ADAMTS13 before the condition or complication is present. In certain embodiments, the subject is administered the composition comprising isolated or recombinant ADAMTS13 after the condition or complication is present.

In certain aspects, the invention provides a method of treating a subject at risk of developing at least one condition or complication associated with SARS-CoV-2 infection or COVID-19, comprising administering a composition comprising isolated or recombinant ADAMTS13. In certain embodiments, the subject is administered the composition comprising isolated or recombinant ADAMTS13 before the condition or complication is present. In certain embodiments, the subject is administered the composition comprising isolated or recombinant ADAMTS13 after the condition or complication is present.

In certain aspects, the invention provides a method of treating or preventing at least one condition or complication in a subject infected with SARS-CoV-2 or suffering from COVID-19, comprising the steps of: a) administering to the subject in need thereof a therapeutically effective amount of ADAMTS13, wherein said therapeutically effective amount is sufficient to: i) reduce circulating ultra-high molecular weight (UHMW) von Willebrand factor (VWF) multimers to a level that is at least about 5%, 10%, 15%, 20%, 25%, 30%, 35%, 40%, 45%, 50%, 55%, 60%, 65%, 70%, 75%, 80%, 85%, 90%, 95%, or 100% decreased compared to a measured level of VWF in the subject's blood prior to administration; ii) reduce circulating UHMW VWF multimers to a level that is no more than about 5%, 10%, 15%, 20%, 25%, 30%, 35%, 40%, 45%, 50%, 55%, 60%, 65%, 70%, 75%, 80%, 85%, 90%, 95%, or 100% above a normal VWF baseline value; iii) reduce circulating VWF to a level that is at least about 5%, 10%, 15%, 20%, 25%, 30%, 35%, 40%, 45%, 50%, 55%, 60%, 65%, 70%, 75%, 80%, 85%, 90%, 95%, or 100% decreased compared to a measured level of VWF in the subject's blood prior to administration; iv) reduce circulating VWF to a level that is no more than about 5%, 10%, 15%, 20%, 25%, 30%, 35%, 40%, 45%, 50%, 55%, 60%, 65%, 70%, 75%, 80%, 85%, 90%, 95%, or 100% above a normal VWF baseline value; v) reduce VWF activity level to a level that is at least about 5%, 10%, 15%, 20%, 25%, 30%, 35%, 40%, 45%, 50%, 55%, 60%, 65%, 70%, 75%, 80%, 85%, 90%, 95%, or 100% decreased compared to a measured level of VWF activity in the subject's blood prior to administration; vi) reduce VWF activity level to a level that is no more than about 5%, 10%, 15%, 20%, 25%, 30%, 35%, 40%, 45%, 50%, 55%, 60%, 65%, 70%, 75%, 80%, 85%, 90%, 95%, or 100% above a normal VWF activity baseline value; vii) increase circulating ADAMTS13 levels from about 100% to about 150% above a normal ADAMTS13 baseline value; or viii) combinations of i)-vii). In certain embodiments, the method further comprises periodically monitoring and adjusting the administered amount to maintain the reduced level of circulating VWF, UHMW VWF multimers, or combinations thereof.

In certain embodiments, the therapeutically effective amount or dose of isolated or recombinant ADAMTS13 is expressed as the number of International Units (IU) of ADAMTS13 activity to be administered, per kg of a subject's body weight (IU/kg). In certain embodiments a therapeutically effective amount or dose will be in the range of about 10-400 IU/kg, about 10-200 IU/kg, preferably about 10-160 IU/kg or 20-160 IU/kg.

Regarding dose, in certain embodiments the therapeutically effective amount of ADAMTS13 is about 10-400 IU/kg, about 10-320 IU/kg, about 10-300 IU/kg, about 10-200 IU/kg, about 10-180 IU/kg, about 10-160 IU/kg, about 20-400 IU/kg, about 20-320 IU/kg, about 20-300 IU/kg, about 20-200 IU/kg, about 20-180 IU/kg, or about 20-160 IU/kg. In further embodiments, the dose is about 10-100 IU/kg, about 10-80 IU/kg, about 10-60 IU/kg, about 10-40 IU/kg, about 10-30 IU/kg, about 10-20 IU/kg, about 20-100 IU/kg, about 20-80 IU/kg, about 20-60 IU/kg, or about 20-40 IU/kg. In yet other embodiments, the dose is about 30-400 IU/kg, about 30-320 IU/kg, about 30-300 IU/kg, 30-180 IU/kg, about 30-160 IU/kg, about 30-150 IU/kg, about 30-80 IU/kg, about 30-60 IU/kg, about 30-40 IU/kg, about 40-400 IU/kg, about 40-320 IU/kg, about 40-300 IU/kg, about 40-180 IU/kg, about 40-160 IU/kg, about 40-150 IU/kg, about 40-80 IU/kg or about 40-60 IU/kg. In certain embodiments, the therapeutically effective amount of ADAMTS13 is about 10-60 IU/kg, about 10-40 IU/kg, or about 10-20 IU/kg. In certain embodiments, the therapeutically effective amount of ADAMTS13 is about 40-320 IU/kg, about 40-160 IU/kg, about 40-80 IU/kg or about 40-60 IU/kg. A therapeutically effective amount or dose may be administered as a single dose, as multiple doses, or as divided doses. In certain embodiments, a therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered once daily, twice daily, or every other day. In certain embodiments, a therapeutically effective amount or dose is administered intravenously, subcutaneously, via intravenous bolus, or via intravenous infusion.

Certain COVID-19 subjects may be over 65 years of age and/or may present with a history, signs, or symptoms of, or a predisposition or susceptibility to, one or more complications or risk factors associated with COVID-19. These include, for example and not limitation, elevated plasma levels of VWF and/or its multimers (especially ultralarge multimers (UHMW)), elevated plasma VWF activity levels, reduced plasma levels of endogenous ADAMTS13, reduced activity of endogenous ADAMTS13, coagulopathy, blood-clotting disorder, veno-occlusive disorder, prothrombotic condition, inherited thrombotic thrombocytopenic purpura (TTP), acquired TTP, disseminated intravascular coagulation (DIC), sepsis, sickle cell, renal failure, respiratory failure, acute respiratory distress syndrome (ARDS), chronic obstructive pulmonary disease (COPD), pneumonia, asthma, pregnancy, menopause, pen-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), cough, shortness of breath, pulmonary infiltrates, respiratory failure, elevated plasma fibrogen, activated hemostasis pathway, intensive care unit (ICU) admission, or symptoms or complications thereof. These conditions are referred to as “risk factors” and such subjects are referred to as subjects or patients “at risk.”

Certain “at risk” COVID-19 subjects may present at earlier stages of the disease, with or without COVID-19 symptoms, for example upon testing the subject for a SARS-CoV-2 infection. These subjects may or may not exhibit elevated levels of VWF and/or its multimers; such levels may appear normal or only slightly elevated. According to the invention, such “at risk earlier stage” subjects, e.g., who exhibit a predisposition or susceptibility to a risk factor, may be treated by administering a composition comprising a therapeutically effective amount of isolated or recombinant ADAMTS13. In certain embodiments, a therapeutically effective amount would be about 10-100 IU/kg, about 10-80 IU/kg, about 10-60 IU/kg, about 10-40 IU/kg, about 10-30 IU/kg, about 10-20 IU/kg, about 20-100 IU/kg, about 20-80 IU/kg, about 20-60 IU/kg, about 20-40 IU/kg, about 20-30 IU/kg, about 30-100 IU/kg, about 30-80 IU/kg, about 30-60 IU/kg, about 30-40 IU/kg, about 40-100 IU/kg, about 40-80 IU/kg, or about 40-60 IU/kg. In certain embodiments, dosing is determined and/or monitored to provide an increase in the subject's circulating ADAMTS13 levels of from about 20-100%, compared to a predetermined normal baseline range (e.g. above the normal range) or predetermined normal baseline value (e.g. above the normal value or reference). The normal range for ADAMTS13 depends upon the method used to measure ADAMTS13 levels or activity. In certain embodiments, the predetermined baseline is based on a normal control population in the testing laboratory with the validated/chosen methods of measurement. In certain embodiments, the normal or baseline range in healthy individuals lies between about 40-160% of the predetermined baseline value. See, Peyvandi et al., ADAMTS13 assays in thrombotic thrombocytopenic purpura, J Thromb Haemost. 2010 April; 8(4):631-40. In certain embodiments, the normal or baseline range in healthy individuals lies between 87-113% of the predetermined baseline value. See, Mancini et al., J Thromb Haemost. 2021 February; 19(2):513-521. In certain embodiments, the composition is administered to “at risk earlier stage” subjects promptly upon detection of SARS-CoV-2, a COVID-19 diagnosis, or hospitalization, or within 24 or 48 hours of detection of SARS-CoV-2, a COVID-19 diagnosis, or hospitalization. In certain embodiments, a therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered once daily, twice daily, or every other day, preferably every other day. According to the invention, treatment as described herein would treat, inhibit, suppress, prevent, reduce, or alleviate severe progression of one or more COVID-19 complications, promote recovery, or maintain a healthier state, particularly regarding the various blood disorders and thrombotic or prothrombotic conditions and complications described herein.

Certain “at risk” COVID-19 patients may present at later stages of the disease, and/or may present with elevated levels of VWF or its multimers (e.g., UHMW VWF multimers). In certain embodiments, elevated VWF or multimer (e.g., UHMW VWF multimers) levels indicating treatment would be levels that are at least about 5%, 10%, 20%, 30%, 40%, 50%, 60%, 70%, 80%, 90%, 100%, 200%, 300%, 400%, 500%, 600%, 700%, 800%, 900% above a predetermined normal baseline value or predetermined baseline range, or higher. VWF is an acute phase protein which also has the tendency to increase with age. There are also different assays measuring different functionalities of VWF. In certain embodiments, the predetermined normal baseline is based on a normal control population in the testing laboratory with the validated/chosen methods of measurement. In certain embodiments, a typical normal baseline range is a range of about 50-200%, compared to an established or predetermined normal reference or predetermined baseline set at 100%. See, Swystun L L, Lillicrap D. J Thromb Haemost. 2018 December; 16(12):2375-2390. In certain embodiments, the normal baseline for VWF levels is one of a VWF:antigen range of about 42-136% or a VWF:activity of about 42-168%. In certain embodiments, treatment is particularly indicated when VWF and/or multimer levels are twice or three times as high, or higher, compared to the normal baseline. According to the invention, “at risk later stage” subjects may be treated by administering a composition comprising a therapeutically effective amount of isolated or recombinant ADAMTS13. In certain embodiments, a therapeutically effective amount would be about 30-200 IU/kg, about 30-180 IU/kg, about 30-160 IU/kg, about 30-80 IU/kg, about 30-60 IU/kg, about 30-40 IU/kg, about 40-180 IU/kg, about 40-160 IU/kg, about 40-80 IU/kg, or about 40-60 IU/kg. In certain embodiments, dosing is determined and/or monitored to provide a reduction in levels of VWF and/or its multimers that are within or approximate a normal range or baseline. In certain embodiments, ultralarge VWF multimers will no longer be observed. In certain embodiments, the ADAMTS13 composition is administered to “at risk later stage” subjects promptly upon detection of SARS-CoV-2, a COVID-19 diagnosis, or hospitalization, or within 24 or 48 hours of detection of SARS-CoV-2, a COVID-19 diagnosis, or hospitalization. In certain embodiments, a therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered daily or every other day, preferably daily. According to the invention, treatment as described herein would treat, inhibit, suppress, prevent, reduce, or alleviate severe progression of one or more COVID-19 complications, promote recovery, or maintain a healthier state, particularly regarding the various blood disorders and thrombotic or prothrombotic conditions and complications described herein.

In certain embodiments, the predetermined normal or established baseline is based on a normal control population in the testing lab with the validated/chosen methods of measurement.

Without wishing to be bound by any theory, it is believed that “at risk later stage” COVID-19 subjects would tend to benefit from administration of higher doses of ADAMTS13, administered more frequently, than doses administered to “at risk earlier stage” COVID-19 subjects. A course of treatment for “at risk earlier stage patients” may be more prolonged than a course of treatment for “at risk later stage patients.” Depending on results and prognosis, these treatments may alternate, or one treatment may be switched to the other.

In certain embodiments, a pharmaceutical composition according to the invention is a stabilized formulation of ADAMTS13 (A13) comprising (a) 0.05 mg/ml to 10.0 mg/ml ADAMTS13; (b) 0 mM to 200 mM of a pharmaceutically acceptable salt; (c) 0.5 mM to 20 mM calcium; (d) a sugar and/or sugar alcohol; (e) a nonionic surfactant; and (f) a buffering agent for maintaining a pH between 6.0 and 8.0. In related embodiments, this is a liquid formulation. In further embodiments, this liquid formulation is lyophilized.

The compositions and methods of the invention will be useful for treating a subject diagnosed with COVID-19, in its earlier stages (e.g. prophylactically) and in its later stages (e.g. as a rescue treatment). The invention will be useful in various aspects, examples of which are described herein. In certain aspects, the invention will be particularly advantageous for: (a) subjects at least 65 years old; (b) subjects exhibiting elevated, abnormally high, or very abnormally high levels of VWF and/or its multimers; (c) subjects exhibiting elevated, abnormally high, or very abnormally high levels of one or more cytokines (e.g. IL-8, TNF-α, and IL-6); (d) subjects exhibiting or at risk for one or more COVID-19 complications, particularly coagulopathy, blood-clotting disorder, veno-occlusive disorder, prothrombotic condition, inherited thrombotic thrombocytopenic purpura (TTP), acquired TTP, disseminated intravascular coagulation (DIC), sepsis, sickle cell, renal failure, respiratory failure, acute respiratory distress syndrome (ARDS), chronic obstructive pulmonary disease (COPD), pneumonia, asthma, pregnancy, menopause, pen-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), cough, shortness of breath, pulmonary infiltrates, respiratory failure, elevated plasma fibrogen, activated hemostasis pathway, intensive care unit (ICU) admission; and (e) any combinations thereof.

In certain embodiments, each of the ADAMTS13, VWF, and cytokine levels and/or activity in a subject would be determined according to suitable laboratory tests, which are known to persons of ordinary skill in the art.

ADAMTS13 activity may be determined, for example, according to Kokame et al., FRETS-VWF73, a first fluorogenic substrate for ADAMTS13 assay, Br J Haematol 2005; 129: 93-100; Tripodi, et al., Measurement of von Willebrand factor cleaving protease (ADAMTS13): results of an international collaborative study involving 11 methods testing the same set of coded plasmas, J. Thromb Haemost 2004 v2 p 1601-9; Tripodi, et. al., Second international collaborative study evaluating performance characteristics of methods measuring the von Willebrand factor cleaving protease (ADAMTS13), J Thromb Haemost. 2008 September; 6(9): 1534-1541, each of which are incorporated herein in their entirety for all intended purposes.

VWF levels may be determined, for example, according to Turecek et al., Comparative Study on Collagen-Binding Enzyme-Linked lmmunosorbent Assay and Ristocetin Cofactor Activity Assays for Detection of Functional Activity of von Willebrand Factor, Seminars In Thrombosis and Hemostasis, 2002 Vol. 28, No. 2, 149-160; Turecek, et al., A Recombinant Von Willebrand Factor Drug Candidate, Seminars In Thrombosis and Hemostasis, 2010 Vol. 36, No. 5, 510-521, each of which are incorporated herein in their entirety for all intended purposes.

Cytokine levels may be determined according to methods known in the art.

In certain aspects, provided herein is a composition and method for treating a subject infected with SARS-CoV-2 or diagnosed with COVID-19 (collectively, a “COVID-19 subject”) by reducing the level of ultralarge VWF multimers in a COVID-19 subject exhibiting at least one of: (a) abnormally high or supernormal levels of endogenous VWF (e.g., more than about 200% measured by the ristocetin cofactor activity assay); (b) abnormally low or ultralow levels of endogenous ADAMTS13 (e.g., less than about 50%), or (c) an abnormal ratio or balance of endogenous VWF:ADAMTS13 (e.g. ≥4:1, or ≥3:1, or >3:1). The method comprises administering to the subject a composition comprising a therapeutically effective amount of an isolated or recombinant ADAMTS13, e.g. to provide a normal or beneficial level of VWF and/or ADAMTS13, or a normal or beneficial ratio of VWF:ADAMTS13.

In certain aspects, the invention provides a composition and method for treating a COVID-19 subject exhibiting or at risk for a sign, symptom, or risk of infarction, thrombosis, or embolism in a COVID-19 subject. The method comprises administering to the subject a composition comprising a therapeutically effective amount of an isolated or recombinant ADAMTS13 to inhibit or suppress a sign or symptom of infarction, thrombosis, or embolism which is achieved when at least one of VWF activity, VWF:ADAMTS13 activity, or any identified sign or symptom, is significantly reduced (e.g., with reference to a predetermined baseline, threshold, or desired statistical measure), and relative to a control, as can be determined by one of skill in the art. Generally, inhibition or suppression is indicated by a reduction in a VWF or VWF:ADAMTS13 activity, or in an evaluation of a sign or symptom, of about 80%, 70%, 60%, 50%, or 25-1% compared to a control.

In certain aspects, the invention provides a composition and method for treating a COVID-19 subject exhibiting or at risk for a coagulopathy or blood-clotting disorder. The method comprises administering to the subject a composition comprising a therapeutically effective amount of an isolated or recombinant ADAMTS13, to prevent or treat the disorder, e.g. by preventing, reducing, or reversing irregular or abnormal blood clots in arteries and/or veins, and/or symptoms or complications thereof.

In certain aspects, the invention provides a composition and method for treating a COVID-19 subject exhibiting or at risk for an arterial or venal thrombosis. The method comprises administering to the subject a composition comprising a therapeutically effective amount of an isolated or recombinant ADAMTS13, e.g. to treat, prevent, reduce or reverse a thrombosis and/or symptoms or complications thereof. In certain embodiments, the thrombosis is a deep vein thrombosis (DVT).

In certain aspects, the invention provides a composition and method for treating a COVID-19 subject exhibiting or at risk for an embolism. The method comprises administering to the subject a composition comprising a therapeutically effective amount of an isolated or recombinant ADAMTS13, e.g. to treat, prevent, reduce or reverse an embolism thrombosis and/or symptoms or complications thereof. In certain embodiments, the embolism is a pulmonary embolism. In certain embodiments, the embolism leads to renal failure.

In certain aspects, the invention provides a composition and method for treating a COVID-19 subject exhibiting or at risk for a veno-occlusive disorder or prothrombotic condition, such as Acute Respiratory Distress Syndrome (ARDS), Chronic Obstructive Pulmonary Disease (COPD), pneumonia, asthma, hypertension (e.g. pulmonary hypertension), myocardial infarction, and stroke (e.g. ischemic or cerebral stroke). The method comprises administering to the subject a composition comprising a therapeutically effective amount of ADAMTS13, e.g. to treat, prevent, reduce, or reverse a veno-occlusive disorder, prothrombotic condition, or symptoms or complications thereof.

In certain aspects, the invention provides a composition and method for treating a human COVID-19 subject that is at least about 65 years of age. The method comprises administering to the subject a composition comprising a therapeutically effective amount of ADAMTS13, e.g. to treat, prevent, reduce, or reverse any symptom, complication, or risk factor for COVID-19, such as a coagulopathy, blood-clotting disorder, veno-occlusive disorder, prothrombotic condition, ARDS, COPD, pneumonia, asthma, menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof.

In certain aspects, the invention provides a composition and method for treating a COVID-19 subject that exhibits elevated cytokine levels. The method comprises administering to the subject a composition comprising a therapeutically effective amount of ADAMTS13, e.g. to reduce the subject's levels of VWF or VWF multimers. In certain embodiments, cytokine levels are determined by measuring one or more of IL-8, TNF-α, and IL-6. Without wishing to be bound by any theory: (a) it is believed that ADAMTS13, by cleaving VWF multimers, will counteract the tendency of ultralarge VWF to increase when cytokine levels increase; (b) elevated levels of IL-8 and TNF-α may lead to the release of ultralarge VWF from Weibel-Palade bodies; and/or (c) IL-6 may interfere with cleavage of ultralarge VWF by ADAMTS13. In certain embodiments, elevated cytokine levels that would indicate treatment include levels that are at least about 10%, 25%, 50%, 100%, 200%, or 300% above a predetermined normal baseline, or higher. In certain embodiments, the elevated cytokine levels are at least about two or three times higher than normal.

In certain aspects, the invention provides a composition and method for treating a human COVID-19 subject that is at least about 65 years old and exhibits an elevated, abnormally high, or supernormal level of VWF protein, VWF multimers, or ratio of VWF to ADAMTS13 (VWF:A13). The method comprises administering to the subject a composition comprising a therapeutically effective amount of ADAMTS13, e.g. to reduce the subject's levels of VWF or VWF multimers, or to treat, prevent, reduce, or reverse any symptom, complication, or risk factor for COVID-19, such as a coagulopathy, blood-clotting disorder, veno-occlusive disorder, prothrombotic condition, ARDS, COPD, pneumonia, asthma, menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof.

In certain aspects, the invention provides a composition and method for treating a human COVID-19 subject that is (a) at least about 65 years old; (b) exhibits or is at risk for any symptom, complication, or risk factor for COVID-19, such as a coagulopathy, blood-clotting disorder, veno-occlusive disorder, prothrombotic condition, ARDS, COPD, pneumonia, asthma, pregnancy, menopause, pen-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof; and (c) exhibits an elevated, abnormally high, or supernormal level of VWF protein, VWF multimers, or ratio of VWF to ADAMTS13 (VWF:A13). The method comprises administering to the subject a composition comprising a therapeutically effective amount of ADAMTS13, e.g. to reduce the subject's levels of VWF or VWF multimers, or to treat, prevent, reduce, or reverse any symptom, complication, or risk factor for COVID-19, such as a coagulopathy, blood-clotting disorder, veno-occlusive disorder, prothrombotic condition, ARDS, COPD, pneumonia, asthma, menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof.

In certain aspects, the invention provides a method of determining whether a subject diagnosed with COVID-19 is at an increased risk for a thrombotic coagulopathy, said method comprising the steps of: a) measuring in a blood plasma sample one or more of: i) a plasma level of VWF protein; ii) an activity level of VWF in the plasma sample; iii) a plasma level of UHMW VWF protein multimers; iv) a plasma level of ADAMTS13 protein; or v) an activity level of ADAMTS13 protein in the plasma sample; and b) comparing the plasma level(s) or activity level(s) measured in step a) to a baseline range or baseline value for the same plasma level(s) or activity level(s); and c) identifying the subject being at risk for a thrombotic coagulopathy when at least one of the following is met: i) the plasma level of VWF protein is increased; ii) the activity level of VWF is increased; iii) plasma UHMW VWF protein multimers are detected or the plasma level of UHMW VWF protein multimers is increased; iv) the plasma level of ADAMTS13 protein is decreased; or v) the activity level of ADAMTS13 protein is decreased, as compared to the baseline range or baseline value for the same plasma level(s) or activity level(s).

In certain embodiments, at least the plasma level of VWF protein is increased. In certain embodiments, at least the activity level of VWF is increased. In certain embodiments, at least UHMW VWF protein multimers are detected or the plasma level of UHMW VWF protein multimers is increased. In certain embodiments, at least the plasma level of ADAMTS13 protein is decreased. In certain embodiments, at least the activity level of ADAMTS13 protein is decreased.

In certain embodiments, thrombotic coagulopathy includes, but is not limited to, platelet aggregation, blood clotting, a thrombosis, a thrombotic microangiopathy, an embolism, an infarction, veno-occlusion, a stroke, renal failure resulting from thrombosis, or combinations thereof. In certain embodiments, the thrombosis is deep vein thrombosis (DVT). In certain embodiments, the embolism is a pulmonary embolism (PE). In certain embodiments, the thrombotic coagulopathy is renal failure resulting from thrombosis.

In certain embodiments, the subject is at risk for developing a thrombotic coagulopathy when the plasma level of VWF protein is about 120% to about 300% of the baseline value for said VWF protein plasma level. In certain embodiments, the subject is at risk for developing a thrombotic coagulopathy when the plasma level of VWF protein is about 300% or more of the baseline value for said VWF protein plasma level. In certain embodiments, the subject is at risk for developing a thrombotic coagulopathy when the activity level of VWF in the plasma sample is about 120% to about 300% of the baseline value for said VWF activity level. In certain embodiments, the subject is at risk for developing a thrombotic coagulopathy when the activity level of VWF in the plasma sample is about 300% or more of the baseline value for said VWF activity level. In certain embodiments, the subject is at risk for developing a thrombotic coagulopathy when the plasma level of ADAMTS13 protein is about 70% to about 100% of the baseline value for said ADAMTS13 protein plasma level. In certain embodiments, the subject is at risk for developing a thrombotic coagulopathy when the plasma level of ADAMTS13 protein is about 70% or less of the baseline value for said ADAMTS13 protein plasma level. In certain embodiments, the subject is at risk for developing a thrombotic coagulopathy when the activity level of ADAMTS13 in the plasma sample is about 70% to about 100% of the baseline value for said ADAMTS13 activity level. In certain embodiments, the subject is at risk for developing a thrombotic coagulopathy when the activity level of ADAMTS13 in the plasma sample is 70% or less of the baseline value for said ADAMTS13 activity level. In certain embodiments, the subject is at risk for developing a thrombotic coagulopathy when the plasma level of UHMW VWF multimers is about 100% to about 110% of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, the subject is at risk for developing a thrombotic coagulopathy when the ratio of VWF:A13 levels in the plasma sample is about 3 or less. In certain embodiments, the subject is at risk for developing a thrombotic coagulopathy when the ratio of VWF:A13 levels in the plasma sample is greater than about 3.

In certain aspects, the invention provides a method of determining whether a subject diagnosed with COVID-19 is at risk for a thrombotic coagulopathy, said method comprising the steps of: a) measuring in a blood plasma sample one or more of: i) a plasma level of VWF protein; ii) an activity level of VWF in the plasma sample; iii) a plasma level of UHMW VWF protein multimers; iv) a plasma level of ADAMTS13 protein; or v) an activity level of ADAMTS13 protein in the plasma sample; and b) identifying the subject being at risk for a thrombotic coagulopathy when at least one of the following is met: i) the plasma level of VWF protein is at least about 1.2 IU/ml; ii) the VWF activity level is at least about 1.2 IU/ml or 1.8 IU/ml; iii) plasma UHMW VWF protein multimers are detected; iv) the plasma level of ADAMTS13 protein is no more than about 0.7 IU/ml; or v) the activity level of ADAMTS13 protein is no more than about 0.8 or about 0.9 IU/ml. In certain embodiments, the method further comprises, administering ADAMTS13. In certain embodiments, the method further comprises, a therapeutically effective amount of the ADAMTS13 is about 10-320 IU/kg, about 10-300 IU/kg, about 10-200 IU/kg, about 10-180 IU/kg, about 10-160 IU/kg, about 10-80 IU/kg, about 10-60 IU/kg, about 10-40 IU/kg, about 10-20 IU/kg, about 20-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, or about 20-30 IU/kg. In certain embodiments, the therapeutically effective amount of the ADAMTS13 is about 10-40 IU/kg, about 10-30 IU/kg, about 10-20 IU/kg, about 20-40 IU/kg, or about 20-30 IU/kg. In certain embodiments, the therapeutically effective amount of the ADAMTS13 is about 10 IU/kg, about 20 IU/kg, about 30 IU/kg, or about 40 IU/kg.

In certain aspects, the invention provides a method of determining whether a subject diagnosed with COVID-19 is at high risk for a thrombotic coagulopathy, said method comprising the steps of: a) measuring in a blood plasma sample one or more of: i) a plasma level of VWF protein; ii) an activity level of VWF in the plasma sample; iii) a plasma level of UHMW VWF protein multimers; iv) a plasma level of ADAMTS13 protein; or v) an activity level of ADAMTS13 protein in the plasma sample; and b) identifying the subject being at high risk for a thrombotic coagulopathy when at least one of the following is met: i) the plasma level of VWF protein is at least about 4.5 IU/ml; ii) the VWF activity level is at least about 3.3 IU/ml or 4.4 IU/ml; iii) the plasma level of ADAMTS13 protein is no more than about 0.4 IU/ml; or iv) the activity level of ADAMTS13 protein is no more than about 0.4 or about 0.5 IU/ml. In certain embodiments, the method further comprises, administering ADAMTS13. In certain embodiments, the method further comprises, a therapeutically effective amount of the ADAMTS13 is about 30-320 IU/kg, about 30-300 IU/kg, about 30-180 IU/kg, about 30-160 IU/kg, about 30-60 IU/kg, about 40-400 IU/kg, about 40-320 IU/kg, about 40-300 IU/kg, about 40-180 IU/kg, about 40-160 IU/kg, about 40-80 IU/kg or about 40-60 IU/kg. In certain embodiments, the therapeutically effective amount of the ADAMTS13 is about 40-400 IU/kg, about 40-320 IU/kg, about 40-300 IU/kg, about 40-180 IU/kg, about 40-160 IU/kg, about 40-80 IU/kg or about 40-60 IU/kg. In certain embodiments, the therapeutically effective amount of the ADAMTS13 is about 40 IU/kg, about 60 IU/kg, about 80 IU/kg, or about 160 IU/kg.

In certain embodiments, the baseline value is a predetermine value based on a normal control population. In certain embodiments, the baseline value is a mean of a predetermine range of a normal control population.

In certain embodiments, the VWF activity level is measured by VWF ristocetin co-factor activity. In certain embodiments, the VWF activity level is measured by VWF collagen binding activity. In certain embodiments, the ADAMTS13 activity level is measured by ELISA. In certain embodiments, the VWF activity level is measured by FRETS.

In certain embodiments, the subject is diagnosed with COVID by detecting SARS-CoV-2 RNA by PCR from a blood or nasal mucus sample taken from the subject. In certain embodiments, the subject is diagnosed with COVID by SARS-CoV-2 seroconversion. In certain embodiments, the subject is diagnosed with COVID by detection of SARS-CoV-2 antibodies in the subject's plasma.

In certain embodiments, the blood sample is treated with an anticoagulant. In certain embodiments, the anticoagulant is EDTA, sodium citrate, or heparin.

In certain aspects, the invention provides a kit for determining whether a subject diagnosed with COVID-19 is at risk for a thrombotic coagulopathy, said kit comprising (i) one or more reagents for determining one or more of the plasma level of VWF protein, activity level of VWF, plasma level of UHMW VWF multimers, plasma level of ADAMTS13 protein, activity level of ADAMTS13, (ii) optionally packaging and/or instructions for use, and (iii) optionally one or more reagents for detecting SARS-CoV-2 or diagnosing COVID-19.

### DESCRIPTION OF THE DRAWINGS

FIGS. 1A-1C shows the alignment between wild-type ADAMTS13 (SEQ ID NO: 1) and ADAMTS13 Q97R variant (SEQ ID NO: 2).

FIG. 2A shows the level of VWF in plasma samples from COVID-19 patients, measured as VWF:UHMVW; VWF:Ag; VWF:CB; and VWF:RCo. FIG. 2B shows levels of ADAMTS13 in the same patients, measured by ELISA, FRETS, and antigen content. Assay results from all patient samples and controls. COVID-19 patient samples (circles); Acute phase TTP control (triangle); Normal Plasma control (squares) and grey boxes represent reference interval derived from historical healthy control populations.

FIG. 3 shows the electrophoretic analysis of VWF multimers from patients with severe COVID-19. COVID-19 plasma samples (B17-B25) were separated by electrophoresis on a 1% agarose gel. VWF multimers were visualized after immunostaining with an anti-VWF primary antibody followed by a secondary goat-anti-rabbit ALP conjugate. Normal control 1—pooled normal plasma after virus inactivation by heat treatment; Normal control 2—pooled normal plasma without heat inactivation; Normal control 3—volunteer healthy donor travelling control plasma; Acute TTP control—sample from a patient with acute autoimmune TTP. The solid lines indicate the fastest migrating band in each lane which corresponds to VWF dimers. The broken lines indicate the upper limit of the stainable part of each lane indicating the largest VWF multimers.

FIGS. 4A-4C show the incubation of plasma from a COVID-19 patient with 0.5 U/ml or 1.0 U/ml of rADAMTS13 compared to an untreated control. For data graphically represented in FIG. 4A, plasma from illustrative severe COVID-19 patient S12 (VWF/ADAMTS13 ratio of 7.4) was incubated with 0.5 IU/mL (triangles) or 1 IU/mL (squares) rADAMTS13 or without ADAMTS13 (diamonds). Sub-samples were taken immediately after addition of rADAMTS13, and at 2 and 5 h and analyzed for VWF activity by the collagen binding assay. FIG. 4B shows pooled absolute VWF:CB results from plasma samples from 10 severe COVID-19 patients labelled as in FIG. 4A. FIG. 4C shows pooled VWF:CBA values labelled as in FIG. 4A but with values expressed as a percentage of the value at 0 h. For both of the pooled data, the points are means and the error bars are standard deviations. There was a significant decrease in VWF:CBA (P<0.001; t-value −7.04) after incubation for 5 h compared with baseline values.

FIGS. 5A-5C show the incubation of plasma from another COVID-19 patient with 0.5 U/ml or 1.0 U/ml of rADAMTS13 compared to an untreated control. FIG. 5B shows a gel patterns of VWF multimers is this patient. Plasma from severe COVID-19 patient B1 (VWF/ADAMTS13 ratio of 13.4) was incubated with 1.0 IU/mL (triangles) or 10.0 IU/mL (squares) rADAMTS13 or without ADAMTS13 (diamonds). Samples were taken immediately after addition of rADAMTS13, and at 2 and 5 hours. FIG. 5A shows VWF activity by the collagen binding assay. FIG. 5B shows multimer composition visualized using the semi-automated electrophoresis gel system (Sebia). FIG. 5C displays the corresponding gel densitograms with the VWF dimer peaks arrowed and high molecular weight VWF multimers to the right of each trace. The vertical axis represents the intensity of gel staining in arbitrary units. The control samples were normal human plasma, B1 plasma without the addition of any reagents and no incubation, and B1 plasma incubated under identical conditions but without the addition of rADAMTS13.

FIG. 6 shows a representative quantitative determination of VWF multimer size in a patient blood sample, using electrophoresis. The plasma sample from the patient was adjusted to 1 IU/mL VWF:Ag and separated on a 1% agarose gel followed by immunostaining and densitometry. The distance between the top of the separation well and the lowest multimer band (VWF dimers) was assigned a migration value of 1.0. In this example the relative migration distance (Rf) of the largest VWF multimer was 0.192 that of the lowest multimer band. The proportion of the total migration distance of the VWF dimer band that is occupied by larger VWF multimers is therefore 1−Rf, in this example calculated as 1.000−0.192=0.808. The 1−Rf value for normal plasma separated on the same gel was 0.729 (not shown). Therefore, the proportion of the patient sample lane containing VWF multimers (UHMW multimer quantitation parameter) is 111% (0.808/0.729*100=111%) that of control reflecting the UHMW multimers near the top of the lane.

FIG. 7 shows a representative densitometric scan of the sample lane for COVID-19 patient (B24), from a patient with acute autoimmune TTP and for a pooled normal plasma sample. The COVID-19 patient had larger multimers than the normal control but less than a patient with acute TTP. The x axis represents the distance in arbitrary units from the upper end of the separation gel (designated 0.0) and the lowest molecular weight band corresponding to the VWF dimers designated as 1.0. They axis is the optical density.

FIGS. 8A-8B show an electrophoretic pattern of VWF multimers from patients with severe COVID-19. FIG. 8A depicts a semi-automated electrophoresis system. Plasma samples of 9 patients with COVID-19 were separated by semi-automated electrophoresis using the HYDRAGEL von WILLEBRAND FACTOR MULTIMERS kit and a HYDRASYS 2 instrument. Each sample was adjusted to 1 IU VWF:Ag per mL, separated and stained for multimers parallel. Control samples from a patient with acute TTP and a healthy volunteer was applied to the same gel. The broken line indicates the largest stainable part of the normal human plasma control. The migration distances of differently sized ultra-high molecular weight multimers were less pronounced as with the home-cast low resolution gels. Although, this prevented reproducible quantitation by densitometry, the UHMW VWF multimers were clearly evident as abnormal immunostaining material above the dotted line. FIG. 8B depicts a home-cast low resolution gel. Patient samples and controls were separated on home cast low-resolution 1% agarose gels and quantified by densitometry to determine to content of UHMW VWF multimers relative to normal plasma as described in the methods section. Electrophoresis lanes shown here cannot be directly compared as they were run on different gels in contrast to A where all samples were separated on the same gel. Vertical lines indicate which samples were separated next to each other. Plasma of COVID-19 patients, adjusted to 1 IU VWF:Ag per mL, was separated and stained for multimers parallel to acute TTP and normal controls.

FIG. 9A shows the electrophoretic VWF multimer pattern upon incubation of severe COVID-19 plasma with ADAMTS13, using a low-resolution gel. FIG. 9B shows the electrophoretic VWF multimer pattern upon incubation of severe COVID-19 plasma with ADAMTS13, using a high-resolution gel.

FIGS. 10A-10D show the relationship between von Willebrand factor laboratory parameters and ADAMTS13 in the COVID-19 samples. FIGS. 10A-10D display the graphical relationship between the laboratory parameters showing lines of best fit from the correlation analysis using the Pearson test. Correlations were classified as strong (r: 0.7 to 1.0), moderate (r: 0.5 to 0.7), weak (r: 0.3 to 0.5) or no correlation (0 to 0.3).

FIGS. 11A-11J show the incubation of each of the 10 severe COVID-19 plasma samples with two different concentrations of rADAMTS13 (in duplicate) compared to an untreated control across incubation time (hrs.), as determined by collagen binding assay. Plasma was incubated with 0.5 IU/mL (triangles) or 1 IU/mL (squares) rADAMTS13 or without ADAMTS13 (diamonds).

FIGS. 12A-12C show electrophoretic VWF multimer analyses of 8 severe COVID-19 plasma samples after incubation with 1 IU/mL ADAMTS13 for 0 hours and 5 hours (FIGS. 12A-12B) or for 0 hours, 2 hours and 5 hours (FIG. 12C).

FIG. 13A-13C shows the alignment between wildtype ADAMTS13 (SEQ ID NO: 1) and wildtype gorilla ADAMTS13 (SEQ ID NO: 3).

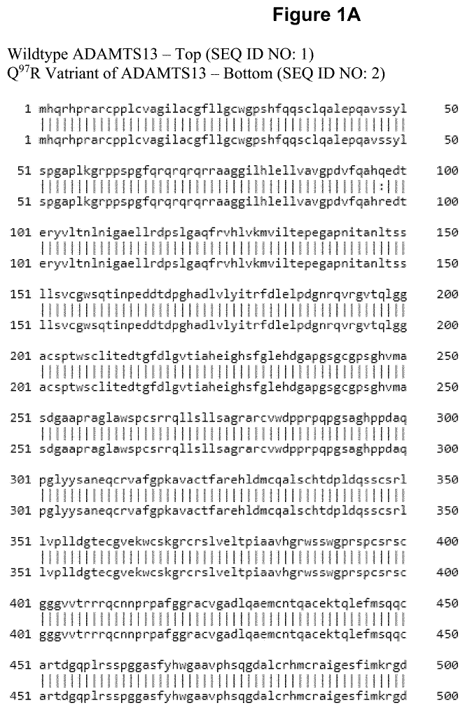

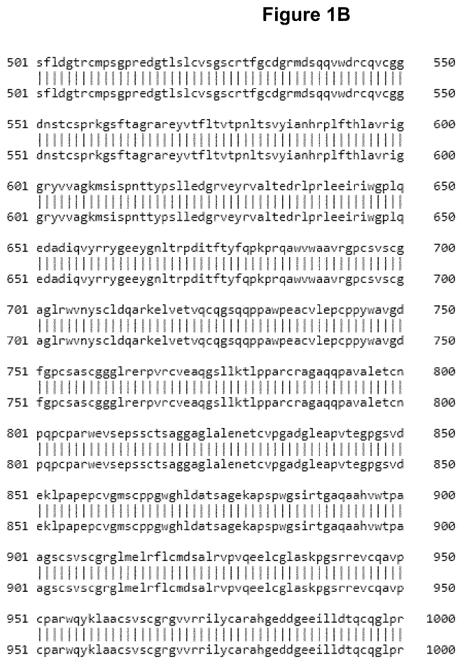

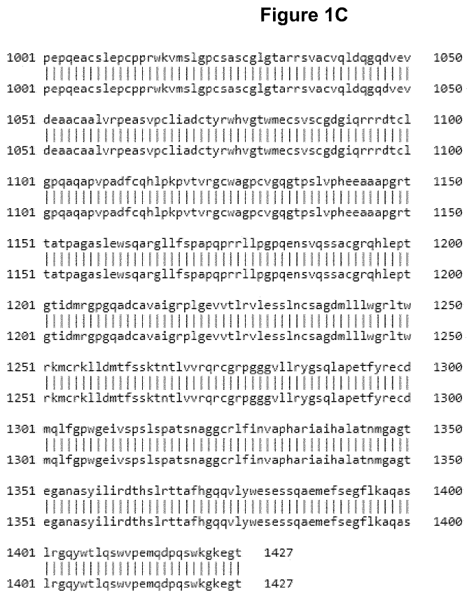

### DETAILED DESCRIPTION

Provided herein are methods for treating a subject suffering from a coronavirus infection, such as a subject infected with a SARS-CoV-2 virus and exhibiting one or more signs or symptoms of SARS-CoV2 infection or COVID-19. More particularly, the inventors have discovered that ADAMTS13 (A Disintegrin-like And Metalloprotease with Thrombospondin type I motif No. 13), in an effective amount, is useful as a treatment for one or more symptoms, risks, or complications of COVID-19. These include, for example and not for limitation, anemic or hemorrhagic infarction, venal or arterial thrombosis, clotting disorders, pulmonary embolism, deep vein thrombosis (DVT), high levels of von Willebrand Factor (VWF), cytokine storm, or any complication or risk factor described herein. ADAMTS13 advantageously exerts its effect in a dose dependent manner. The composition comprises a therapeutically effective amount of an isolated or recombinant (ADAMTS13) protein. The method includes a step of administering to the subject a therapeutically effective amount of an isolated or recombinant ADAMTS13, including embodiments that provide particular dosages at particular times.

ADAMTS13 is a member of the ADAMTS protein family, which includes metalloproteinases containing a number of conserved domains, including a zinc-dependent catalytic domain, a cysteine-rich domain, a disintegrin-like domain, and at least one, and in most cases multiple, thrombospondin type I repeats. See, Nicholson et al., BMC Evol Biol. 2005 Feb. 4; 5(1):11). These proteins are evolutionarily related to the ADAM and MMP families of metalloproteinases. (Jones G C, Curr Pharm Biotechnol. 2006 February; 7(1):25-31). They are secreted enzymes that have been linked to a number of diseases and conditions including thrombotic thrombocytopenic purpura (TTP) (Moake J L, Semin Hematol. 2004 January; 41(1):4-14), connective tissue disorders, cancers, inflammation (Nicholson et al.), and severe Plasmodium falciparum malaria (Larkin et al., PLoS Pathog. 2009 March; 5(3):e1000349). Because of these associations, the ADAMTS enzymes have been recognized as potential therapeutic targets for a number of pathologies (Jones G C, Curr Pharm Biotechnol. 2006 February; 7(1):25-31). Loss of ADAMTS13 activity has been linked to a number of conditions, such as TTP (Moake J L, Semin Hematol. 2004 January; 41(1):4-14), acute and chronic inflammation (Chauhan et al., J Exp Med. 2008 Sep. 1; 205(9):2065-74), and severe Plasmodium falciparum malaria (Larkin et al., PLoS Pathog. 2009 March; 5(3):e1000349).

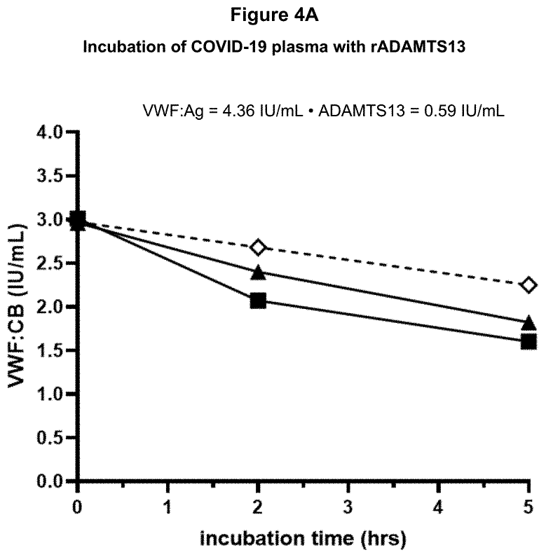

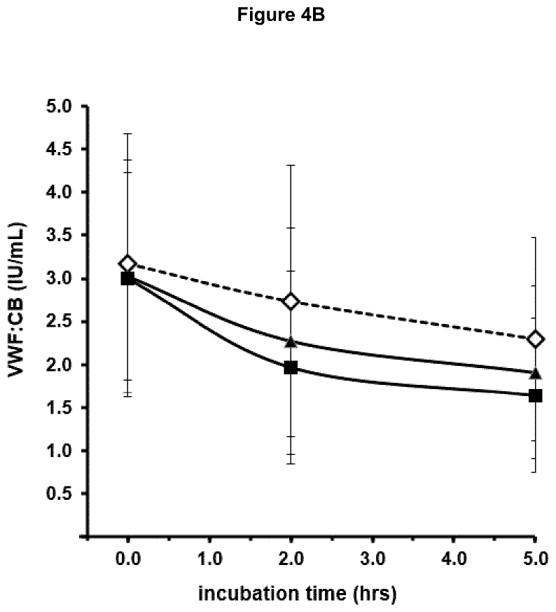

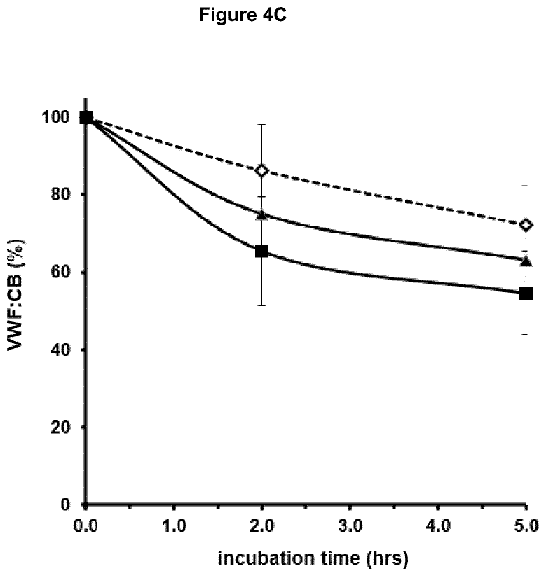

The ADAMTS13 protease is a 190 kDa glycosylated protein produced predominantly by the liver (Levy G G, et al., Nature. 2001; 413:488-494; Fujikawa K, et al., Blood. 2001; 98:1662-1666; Zheng X, et al., J Biol Chem. 2001; 276:41059-41063; Soejima K, et al., J Biochem (Tokyo). 2001; 130:475-480; Gerritsen H E et al., Blood. 2001; 98:1654-1661, each of which is incorporated herein by reference in their entirety for all purposes). ADAMTS13 is expressed as a precursor with an N-terminal propeptide. The mature ADAMTS13 comprises a metalloprotease (M) domain, a disintegrin-like (D) domain, a thrombospondin type1 (T) repeat, a cysteine-rich (C) domain, and a spacer (S) domain, followed by seven consecutive TSP1 repeats (T2-T8) and two CUB domains as illustrated in FIG. 4. Structural information of different domains has been reported on ADAMTS family proteins, including a structure of human ADAMTS13 DTCS (residues 287-685) (Akiyama M., Takeda S., Kokame K., Takagi J., Miyata T. 2009 Crystal structures of the noncatalytic domains of ADAMTS13 reveal multiple discontinuous exosites for von Willebrand factor, Proceedings of the National Academy of Sciences 106: 19274-19279, which is incorporated herein by reference in its entirety for all purposes). Structural analyses indicate that ADAMTS family members share sequence conservation and structure similarity of MDTCS domains (Akiyama et al., 2009 supra; Mosyak L., Georgiadis K., Shane T., Svenson K. et al. 2008 Crystal structures of the two major aggrecan degrading enzymes, ADAMTS4 and ADAMTS5, Protein Science 17: 16-21, each of which is incorporated herein by reference in their entirety for all purposes).

VWF synthesized in megakaryocytes and endothelial cells is stored in platelet granules and Weibel-Palade bodies, respectively, as ultralarge VWF multimers (UHMW or UL-vWF). Moake et al., N Engl J Med. 1982; 307:1432-1435; Wagner et al., J Cell Biol. 1982; 95:355-360; Wagner et al., Mayo Clin Proc. 1991; 66:621-627; Sporn et al., Blood. 1987; 69:1531-1534; Tsai et al., Biochem Biophys Res Commun. 1989; 158:980-985; Tsai et al., Blood. 1989; 73:2074-2076. Once secreted from endothelial cells, these UL-vWF multimers are cleaved by ADAMTS13 in circulation into a series of smaller multimers at specific cleavage sites within the vWF molecule. See, Tsai et al., Biochem Biophys Res Commun. 1989; 158:980-985; Dent et al., J Clin Invest, 1991; 88:774-782; Furlan et al., Proc Natl Acad Sci USA. 1993; 90:7503-7507.

ADAMTS13 cleaves at the Tyr842-Met843 bond in the central A2 domain of the mature vWF subunit and requires zinc or calcium for activity. Dent et al., Proc Natl Acad Sci USA, 1990; 87:6306-6310). The VWF-proteolytic activity of ADAMTS13 is highly dependent on divalent cations, which has also been observed in other the metalloprotease domains in this ADAMTS family (Zheng et al., 2001 supra; Gardner M. D., Chion C. K., de Groot R., Shah A., Crawley J. T. et al. 2009 A functional calcium-binding site in the metalloprotease domain of ADAMTS13, Blood 113: 1149-1157, which is incorporated herein by reference in its entirety for all purposes). Moreover, ADAMTS13 activity undergoes allosteric regulation by binding to VWF and interactions between N-terminal MDTCS and C-terminal CUB domains (Muia J., Zhu J., Gupta G., Haberichter S. L., Friedman K. D. et al. 2014 Allosteric activation of ADAMTS13 by von Willebrand factor, Proceedings of the National Academy of Sciences 111: 18584-18589; South K., Luken B. M., Crawley J. T. B., Phillips R., Thomas M., et al. 2014 Conformational activation of ADAMTS13, Proceedings of the National Academy of Sciences 111: 18578-18583, each of which is incorporated herein by reference in their entirety for all purposes).

Occurrence of supra-physiological levels of VWF, induced by COVID-19, can consume ADAMTS13 by substrate overload, which in turn may lower active levels of native circulating ADAMTS13 to a concentration below a critical threshold. Administration of exogenous isolated or recombinant ADAMTS13 can counteract this dynamic, restore the balance of ADAMTS13 and VWF, and therapeutically reduce the level or concentration of VWF, especially ultralarge VWF proteins. In this way, ADAMTS13 reduces the stickiness of VWF to platelets and avoids or reduces the formation of platelet thrombi, or contributes to dissolving, removing, or alleviating thrombi which may form or be forming. Administration of therapeutically effective amounts of ADAMTS13 in turn can reduce or eliminate clotting disorders, thrombosis, embolism, and other complications and risk factors in COVID-19 subjects. In certain embodiments, the disorder is marked by elevated levels of VWF in the bloodstream. In certain embodiments, the thrombosis is deep vein thrombosis (DVT). In certain embodiments the embolism is pulmonary embolism. In certain embodiments, ADAMTS13 therapy according to the invention is suitable for severe or late-stage cases of COVID-19, including treatment of critically ill patients. Further, administration of ADAMTS13 is well-tolerated and has little risk of side effects.

### I. Definitions

As used herein, “ADAMTS13” or “A13” refer to a metalloprotease of the ADAMTS (a disintegrin and metalloproteinase with thrombospondin type I motifs) family, i.e., No. 13, that cleaves von Willebrand factor (VWF) between residues Tyr 1605 and Met 1606. In the context of the present invention, an ADAMTS13 embraces any ADAMTS13 protein, for example, ADAMTS13 from a mammal such as a primate, human (NP 620594), monkey, rabbit, pig, bovine (XP 610784), rodent, mouse (NP 001001322), rat (XP 342396), hamster, gerbil, canine, feline, frog (NP 001083331), chicken (XP 415435), and biologically active derivatives or fragments thereof. As used herein, “ADAMTS13 proteins” refer to recombinant and isolated or plasma derived ADAMTS13 proteins, variants, or derivatives or fragments thereof. In certain embodiments, the ADAMTS13 is wildtype human ADAMTS13 (hADAMTS13) or fragment thereof as described in U.S. Patent Application Publication No. 2012/0229455, which is incorporated herein by reference for all purposes. In certain embodiments, the amino acid sequence of hADAMTS13 is that of GenBank accession number NP 620594. In certain embodiments, the hADAMTS13 is SEQ ID NO: 1, SEQ ID NO. 2, or a combination or mixture thereof. Mutant and variant ADAMTS13 proteins having activity are also embraced, as are functional fragments and fusion proteins of the ADAMTS13 proteins. Furthermore, the ADAMTS13 proteins of the invention may further comprise tags that facilitate purification, detection, or both. The ADAMTS13 proteins described herein may further be modified with a therapeutic moiety or a moiety suitable imaging in vitro or in vivo.

The term “ADAMTS13 variant” refers to a polypeptide substantially similar in structure and having the same biological activity, albeit in certain instances to a differing degree, to a wildtype molecule (e.g., SEQ ID NO: 1). Variants differ in the composition of their amino acid sequences compared to the wildtype polypeptide from which the variant is derived, based on one or more mutations involving (i) deletion of one or more amino acid residues at one or more termini of the polypeptide (including fragments as described above) and/or one or more internal regions of the wildtype polypeptide sequence, (ii) insertion or addition of one or more amino acids at one or more termini (typically an “addition” variant) of the polypeptide and/or one or more internal regions (typically an “insertion” variant) of the wildtype polypeptide sequence or (iii) substitution of one or more amino acids for other amino acids in the wildtype polypeptide sequence. Substitutions are conservative or non-conservative based on the physico-chemical or functional relatedness of the amino acid that is being replaced and the amino acid replacing it. A variant includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987, incorporated herein by reference for all purposes). In certain embodiments, the ADAMTS13 variant is SEQ ID NO: 2. The term “variant,” in some aspects, is interchangeably used with the term “mutant”.

Human ADAMTS13 proteins include, without limitation, polypeptides comprising the amino acid sequence of GenBank accession number NP 620594 or a processed polypeptide thereof, for example a polypeptide in which the signal peptide (amino acids 1 to 29) and/or propeptide (amino acids 30-74) have been removed. Many natural variants of human ADAMTS13 are known in the art, and are embraced by the compositions of the present invention, some of which include mutations selected from R⁷W, V⁸⁸M, H⁹⁶D, R¹⁰²C, R¹⁹³W, T¹⁹⁶I, H²³⁴Q, A²⁵⁰V, R²⁶⁸P, W³⁹⁰C, R³⁹⁸H, Q⁴⁴⁸E, Q⁴⁵⁶H, P⁴⁵⁷L, P⁴⁷⁵S, C⁵⁰⁸Y, R⁵²⁸G, P⁶¹⁸A, R⁶²⁵H, I⁶⁷³F, R⁶⁹², A⁷³²V, E⁷⁴⁰K, A⁹⁰⁰V, S⁹⁰³L, C⁹⁰⁸Y, C⁹⁵¹G, G⁹⁸²R, C¹⁰²⁴G, A¹⁰³³T, R¹⁰⁹⁵W, R¹⁰⁹⁵W, R¹¹²³C, C¹²¹³Y, T¹²²⁶I, G¹²³⁹V, and R¹³³⁶W. Additionally, ADAMTS13 proteins include natural and recombinant proteins that have been mutated, for example, by one or more conservative mutations at a non-essential amino acid. Preferably, amino acids essential to the enzymatic activity of ADAMTS13 will not be mutated. These include, for example, residues known or presumed to be essential for metal binding such as residues 83, 173, 224, 228, 234, 281, and 284, and residues found in the active site of the enzyme, e.g., residue 225. Similarly, in the context of the present invention, ADAMTS13 proteins include alternate isoforms, for example, isoforms lacking amino acids 275 to 305 and/or 1135 to 1190 of the full-length human protein.

ADAMTS13 proteins may be further modified, for example, by post-translational modifications (e.g., glycosylation at one or more amino acids selected from human residues 142, 146, 552, 579, 614, 667, 707, 828, 1235, 1354, or any other natural or engineered modification site) or by ex vivo chemical or enzymatic modification, including without limitation, glycosylation, modification by water soluble polymer (e.g., PEGylation, sialylation, HESylation, etc.), tagging, and the like.

As used herein, the term “glycosylated” or “glycosylated forms of ADAMTS13” refers to ADAMTS13 proteins that are post-translationally modified by the addition of carbohydrate or glycan residues. An ADAMTS13 protein having more than one glycosylation site can have the same glycan residue attached to each glycosylation site, or can have different glycan residues attached to different glycosylation sites. In this manner, different patterns of glycan attachment can yield different glycoforms of an ADAMTS13 protein. The predominant sugars found on a glycosylated ADAMTS13 are glucose (Glc), galactose (Gal), mannose (Man), fucose (Fuc), N-acetylgalactosamine (GalNAc), N-acetylglucosamine (GlcNAc), and sialic acid (e.g., N-acetyl-neuraminic acid (NeuAc or NANA)). Hexose (Hex) and HexNAc are generic terms that represent classes of monosaccharides such as Man, Glc, and Gal residues, and GlcNAc and GalNAc residues, respectively.

The term “glycosylation” includes the formation of ADAMTS13 glycoproteins where glycan residues are attached either to the side chain of an asparagine (Asn) residue (i.e., N-linked), or a serine (Ser) or threonine (Thr) residue (i.e., O-linked), or a tryptophan (Trp) residue (i.e., C-linked and/or C-mannosylation) of a protein.

The term “N-glycosylation site” refers to any amino acid sequence that includes an amino acid residue having a nitrogen atom, e.g., the amide nitrogen of an asparagine residue. The N-glycans attached to glycoproteins differ with respect to the number of branches (antennae) comprising peripheral sugars (e.g., GlcNAc, Gal, Fuc, and NeuAc) that are added to a common core pentasaccharide: Man3GlcNAc2, that contains a “trimannose” (Man3) component and “chitobiose” (GlcNAc2) component. N-glycans are commonly classified according to their branched constituents (e.g., high mannose, hybrid or complex). A “high-mannose” type N-glycan contains unsubstituted terminal mannose sugars. These glycans typically contain between five and nine mannose residues attached to the chitobiose core. “Hybrid” type N-glycans can contain both unsubstituted terminal mannose residues and substituted mannose residues with a GlcNAc linkage. A “complex” type N-glycan typically has at least one GlcNAc attached to an α1,3 mannose arm and at least one GlcNAc attached to an α1,6 mannose arm of the trimannose core. Complex N-glycans may also have Gal or GalNAc sugar residues that are optionally modified with NeuAc residues. Complex N-glycans may also have intrachain substitutions comprising “bisecting” GlcNAc and core Fuc residues. Complex N-glycans may also have multiple antennae on the trimannose core, often referred to as “multiple antennary glycans.”

“O-linked glycosylation” refers to a form of glycosylation where a carbohydrate residue (e.g., GalNAc, Gal) is added to a hydroxyl amino acid, e.g., serine or threonine. O-linked glycans commonly comprise an O-fucosylation bearing the disaccharide Fuc-Glc or mucin-type structures containing HexNAc-Hex-NeuAco-2. The term “O-glycosylation site” refers to any amino acid sequence that includes an amino acid residue having a hydroxyl group (e.g., serine, threonine or tyrosine side chains).

“C-linked glycosylation” refers to a form of glycosylation where a carbohydrate residue (e.g., Man) is added to a carbon on a tryptophan side chain. The term “C-glycosylation site” or “C-mannosylation site” refers to any amino acid sequence that includes an amino acid residue having a carbon atom, e.g., a carbon atom on a tryptophan side chain.

As used herein, the term “glycosimilarity index” or “glycan index” or “N-glycan index” refers to the conformity degree of a reference glycosylation profile compared to a given target profile.

As used herein, a “biologically active derivative” or “biologically active variant” of ADAMTS13 refers to any polypeptide with substantially the same biological function as ADAMTS13. The polypeptide sequences of the biologically active derivatives may comprise deletions, additions and/or substitution of one or more amino acids whose absence, presence and/or substitution, respectively, do not have any substantial negative impact on the biological activity of polypeptide. The biological activity of said polypeptides may be measured, for example, by the reduction or delay of platelet adhesion to the endothelium, the reduction or delay of platelet aggregation, the reduction or delay of the formation of platelet strings, the reduction or delay of thrombus formation, the reduction or delay of thrombus growth, the reduction or delay of vessel occlusion, the proteolytic cleavage of VWF, the disintegration of thrombi, or by cleavage of a peptide substrate, for example a FRETS-VWF73 peptide. See, Kokame et al., Br J Haematol. 2005 April; 129(1):93-100), or any variant thereof. See also, Tripodi, et al., J. Thromb Haemost 2004 v2 p 1601-9; Tripodi, et. al., J Thromb Haemost. 2008 September; 6(9): 1534-1541, each of which incorporated by reference for all purposes.

Additionally, the terms “ADAMTS13”, “biologically active variant”, and a “biologically active derivative” thereof, include polypeptides obtained via recombinant DNA technology. The recombinant ADAMTS13 (“rADAMTS13”), e.g. recombinant human ADAMTS13 (“r-hu-ADAMTS13”), may be produced by any method known in the art. One specific example is disclosed in WO 02/42441 which is incorporated herein by reference with respect to the method of producing recombinant ADAMTS13. This may include any method known in the art for (i) the production of recombinant DNA by genetic engineering, e.g. via reverse transcription of RNA and/or amplification of DNA, (ii) introducing recombinant DNA into prokaryotic or eukaryotic cells by transfection, i.e. via electroporation or microinjection, (iii) cultivating said transformed cells, e.g. in a continuous or batchwise manner, (iv) expressing ADAMTS13, e.g. constitutively or upon induction, and (v) isolating said ADAMTS13, e.g. from the culture medium or by harvesting the transformed cells, in order to (vi) obtain substantially purified recombinant ADAMTS13, e.g. via anion exchange chromatography or affinity chromatography.

Also included are proteins having ADAMTS13 activity and an amino acid sequence has at least 80% identity, preferably 85%, 90%, 91%, 92%, 93%, 94%, 95%, 96%, 97%, 98%, 99%, or 100% identity to a reference ADAMTS13 sequence, including for example a wild-type ADAMTS13 sequence, the sequence of GenBank accession number NP 620594, or a sequence of SEQ ID NO: 1 or SEQ ID NO: 2.

Also included are chimeric molecules such as, e.g. ADAMTS13 (or a biologically active derivative thereof) in combination with an immunoglobulin (Ig), e.g., an antibody or portion thereof, in order to improve the biological/pharmacological properties such as, e.g; the half-life of ADAMTS13 in the circulation system of a mammal, particularly a human. The Ig could have also the site of binding to an optionally mutated Fc receptor.

As used herein, “ADAMTS13 activity” includes the cleavage of full-length VWF, VWF fragments, or a VWF substrate (e.g., FRETS-VWF73 substrate (Kokame et al., Br J Haematol. 2005 April; 129(1):93-100)). “ADAMTS13 activity” may refer to the activity of ADAMTS13 protein (e.g., wildtype or variant), or combinations or ADAMTS13 proteins. In certain embodiments, when the composition is a mixture of ADAMTS13 variant(s) and/or a ADAMTS13 (e.g., wildtype), “ADAMTS13 activity” refers to the activity of total ADAMTS13 in the composition.

As used herein, “one unit of ADAMTS13 activity” is defined as the amount of activity in 1 ml of pooled normal human plasma, regardless of the assay being used. For example, one unit of ADAMTS13 FRETS-VWF73 activity is the amount of activity needed to cleave the same amount of FRETS-VWF73 substrate as is cleaved by one ml of pooled normal human plasma (a reference or baseline sample). See, Kokame et al., Br J Haematol. 2005 April; 129(1):93-100), or any variant thereof. See also, Tripodi, et al., J. Thromb Haemost 2004 v2 p 1601-9; Tripodi, et. al., J Thromb Haemost. 2008 September; 6(9): 1534-1541, each incorporated herein in their entirety for all purposes. For example, direct ADAMTS13 activity assays can be performed to detect the cleavage of either full-length VWF molecules or VWF fragments using SDS agarose gel electrophoresis and indirect detection of ADAMTS13 activity can be detected with collagen binding assays. The term “one unit of ADAMTS13 activity” can be used interchangeably with “Activity unit”, “U”, “international unit”, “IU”, or “UFV73”. In certain embodiments, the international unit is based on the use of a WHO standard that was calibrated against plasma using the VWF FRETS assay (i.e., “UFV73” or “IU”).

As used herein, the terms “treat” and “prevent” are not intended to be absolute terms. Treatment can refer to any delay in onset, amelioration of symptoms, improvement in subject or patient survival, reduction in frequency or severity, etc. Treatment also encompasses any improvement in the condition or state of a subject, including any sign, symptom, or complications thereof, whether or not the treatment directly or indirectly affects the underlying causes or etiology of a condition or disease. The term “prevent” includes prophylaxis and prevention. The effect of treatment can be compared to a control, e.g., a subject or pool of subjects not receiving the treatment, an untreated tissue in the same patient, or the same subject prior to treatment.

A “subject” or “patient,” as used herein, means any mammal, including a human subject, that exhibits or is at risk for a condition or disease, or any signs, symptoms or complications thereof. A “COVID-19 subject” or “COVID-19 patient” means any mammal, including a human subject, that exhibits signs or symptoms of COVID-19, is diagnosed with COVID-19, or exhibits an infection with SARS-CoV-2, for example by testing positive for SARS-CoV-2 in a clinical or laboratory test.

As used herein, a “therapeutically effective amount or dose” or “sufficient amount or dose” refers to a dose that produces effects for which it is administered. The exact dose will depend on the purpose of the treatment and will be ascertainable by one skilled in the art using known techniques. See, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins.

The term “gene” means the segment of DNA (nucleic acid or polypeptide) involved in producing a polypeptide chain. It can include regions preceding and following the coding region (leader and trailer) as well as intervening sequences (introns) between subject coding segments (exons).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues. See, Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994). The term nucleic acid encompasses a gene, cDNA, and mRNA encoded by a gene.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid. There are various known methods in the art that permit the incorporation of an unnatural amino acid derivative or analog into a polypeptide chain in a site-specific manner, see, e.g., WO 02/086075.

“Conservatively modified variants” applies to both amino acid and nucleic acid sequences. With respect to particular nucleic acid sequences, conservatively modified nucleic acids refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variants. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, “conservatively modified variants” refers to substitutions, deletions or additions to a nucleic acid, peptide, polypeptide, or protein sequence which alters, adds or deletes a single amino acid or a small percentage of amino acids in the encoded sequence, resulting in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are examples of conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). See, e.g., Creighton, Proteins, W. H. Freeman and Co., N. Y. (1984).

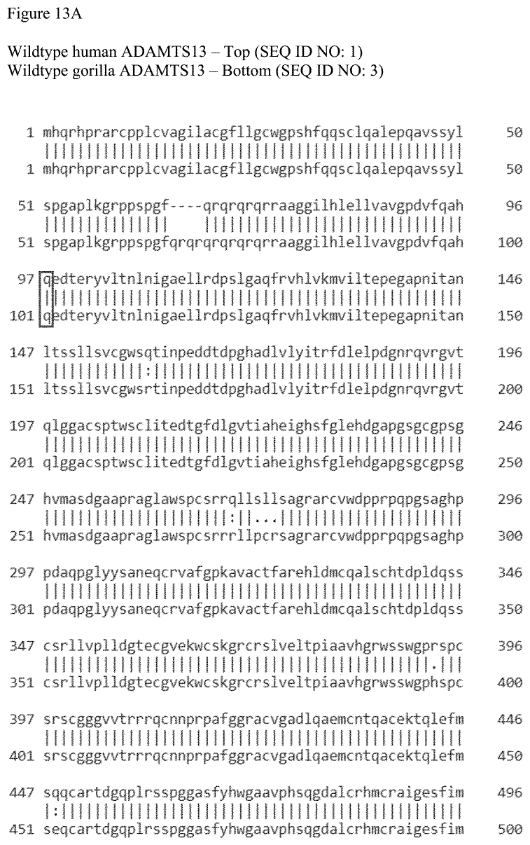

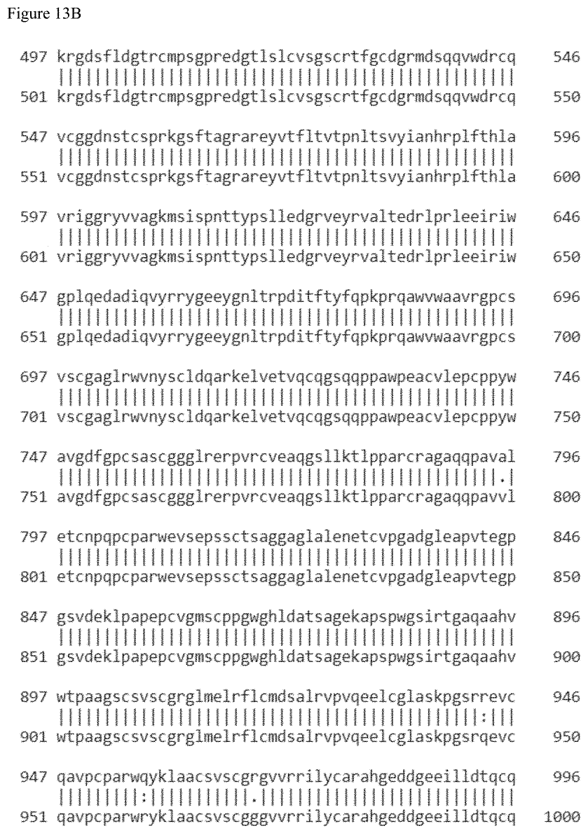

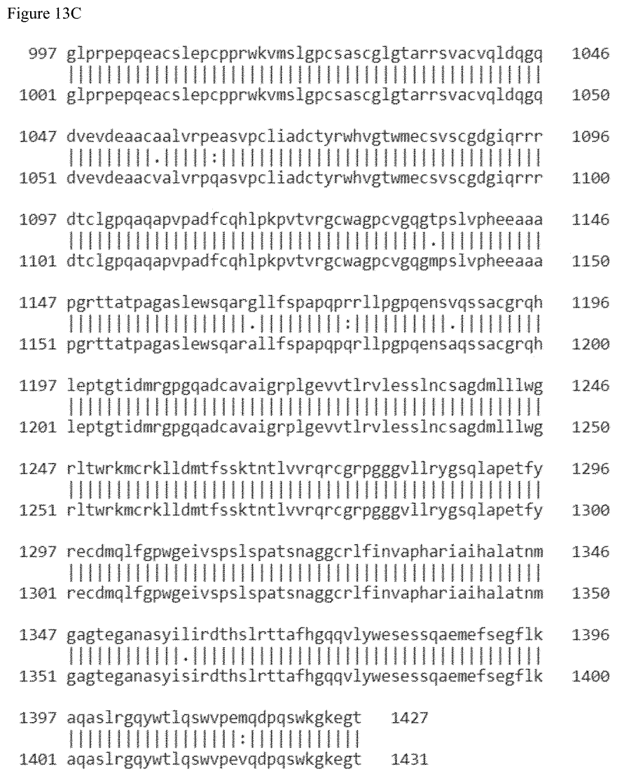

As used herein, an “equivalent position” (for example, an “equivalent amino acid position” or “equivalent residue position”) is defined herein as a position (such as, an amino acid position or a residue position) of an amino acid sequence which aligns with a corresponding position of a reference amino acid sequence (e.g., SEQ ID NO: 1), using an alignment algorithm (e.g., Clustal Needleman-Wunsch algorithm, Vector NTI). The equivalent amino acid position of the amino acid sequence need not have the same numerical position number as the corresponding position of the reference amino acid sequence. As an example, FIG. 13 shows the sequence of a human wildtype ADAMTS13 (SEQ ID NO: 1) aligned with a gorilla wildtype ADAMTS13 (SEQ ID NO: 3). In this example, amino acid position number 97 of SEQ ID NO:1 is considered to be an equivalent amino acid position to (i.e. is “equivalent to”) that of amino acid position number 101 of SEQ ID NO: 3, as amino acid number 97 of SEQ ID NO: 1 aligns with amino acid number 101 of SEQ ID NO: 3. In other words, amino acid position 97 of SEQ ID NO: 1 corresponds to amino acid position 101 of SEQ ID NO: 3.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds. In the present application, the amino acid residues of a polypeptide, peptide, or protein are numbered according to their relative positions from the left most residue, which is numbered 1, for example by reference to an unmodified wild-type polypeptide sequence.

As used herein, a “fragment” of a polypeptide refers to any portion of the polypeptide smaller than the full-length polypeptide or protein expression product. Fragments are typically deletion analogs of the full-length polypeptide, wherein one or more amino acid residues have been removed from the amino terminus and/or the carboxy terminus of the full-length polypeptide. Accordingly, “fragments” are a subset of deletion analogs described below.

The term “recombinant” or “recombinant expression system” when used with reference, e.g., to a cell, indicates that the cell has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example at least 80% identity, preferably 85%, 90%, 91%, 92%, 93%, 94%, 95%, 96%, 97%, 98%, 99%, or 100% identity, to a reference sequence, e.g., SEQ ID NO:1 or SEQ ID NO: 2), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, sub-sequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and default parameters may be used.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

As used herein, phrases “total amount of ADAMTS13” or “total ADAMTS13” in a composition includes the sum total of all ADAMTS13 proteins (e.g., wildtype and variant(s)) in the composition. For example, if a composition comprises ADAMTS13 wildtype and Q⁹⁷R ADAMTS13 variant, the “total amount of ADAMTS13” or “total ADAMTS13” would be the sum total of ADAMTS13 wildtype and Q⁹⁷R ADAMTS13 in the composition. Likewise, if a composition comprises only Q⁹⁷R ADAMTS13, the total amount of ADAMTS13 or total ADAMTS13 would be the sum total of Q⁹⁷R ADAMTS13 in the composition.

A “biological sample” can be obtained from a subject or patient, e.g., a biopsy, from an animal, such as an animal model, or from cultured cells, e.g., a cell line or cells removed from a patient and grown in culture for observation. Biological samples include tissue such as colorectal tissue or bodily fluids, e.g., blood, blood fractions, lymph, saliva, urine, feces, etc.

As used herein, a “physiological concentration” of salt refers to a salt concentration of between about 100 mM and about 200 mM of a pharmaceutically acceptable salt. Non-limiting examples of pharmaceutically acceptable salts include, without limitation, sodium and potassium chloride, sodium and potassium acetate, sodium and potassium citrate, sodium and potassium phosphate.

As used herein, a “sub-physiological concentration” of salt refers to a salt concentration of less than about 100 mM of a pharmaceutically acceptable salt. In preferred embodiments, a sub-physiological concentration of salt is less than about 80 mM of a pharmaceutical salt. In another preferred embodiment, a sub-physiological concentration of salt is less than about 60 mM of a pharmaceutical salt.

As used herein, the term “about” means within an acceptable deviation or error range for the particular variable or value, as determined by one of ordinary skill in the art, which will depend on the context and how the variable or value is measured or determined (such as the limitations of the measurement system). For example, “about” can mean within an acceptable standard deviation, per the practice in the art. Alternatively, “about” can mean a range of up to ±20%, preferably up to ±10%, more preferably up to ±5%, and more preferably still up to ±1% of a given variable or value, again per the practice of the art for that. For example, the stated or indicated dose or dosage range for a drug or pharmaceutical composition may, in practice, vary within acceptable ranges or limits, as understood by a person or ordinary skill in the art. Also, when “about” appears before a range, a list of numbers, or a list of ranges, it should be interpreted that the term “about” appears before each number in that list or range.

If aspects of the disclosure are described as “comprising”, or versions thereof (e.g., comprises), a feature, embodiments also are contemplated “consisting of” or “consisting essentially of” the feature.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

### II. Compositions and Methods

A. Recombinant ADAMTS13 Proteins

The subject methods provided herein include a step of administering to a subject infected with SARS-CoV2 or diagnosed with COVID-19 a pharmaceutical composition that includes a therapeutically effective amount of an isolated or recombinant ADAMTS13 protein.

The human ADAMTS13 gene contains 29 exons and spans approximately 37 kb on chromosome 9q34. The 4.7 kb transcript is predominantly synthesized in hepatic stellate cells, but also in vascular endothelial cells and platelets and encodes a primary translation product of 1427 amino acid residues. The precursor ADAMTS13 polypeptide consists of a signal peptide and a propeptide that C-terminally ends in a potential furin site for cleavage, followed by the sequence of the mature VWF-cleaving protease. The mature ADAMTS13 polypeptide (1353 amino acid residues) comprises the structural features characteristic of all ADAMTS family members: a reprolysin-like metalloprotease domain, a disintegrin-like domain, a central thrombospondin type 1 (TSP1) repeat, a cysteine-rich domain harboring a RGD motif possibly important for integrin interactions, and a spacer domain, thereafter followed by a unique combination of 7 consecutive TSP1 repeats (TSP1/#2-8) and two CUB domains.

The mature ADAMTS13 has a calculated molecular mass of about 145 kDa whereas purified plasma-derived ADAMTS13 has an apparent molecular mass of about 180 kDa probably due to post-translational modifications consisting with present consensus sequences for 10 potential N-glycosylation sites, and several O-glycosylation sites and one C-mannosylation site in the TSP1 repeats. The VWF-proteolytic activity of ADAMTS13 is highly dependent on divalent cations. The active site motif in the metalloprotease domain contains the highly conserved HEXXHXXGXXHD motif with three histidine residues that coordinate a catalytic Zn2+ ion and a predicted Calcium binding site proposed to be coordinated by Glu 83, Asp 173, Cys 281 and Asp 284. The functional roles of the ADAMTS13 domains have been studied mainly using in vitro assay systems, showing that the N-terminal regions from the metalloprotease to the spacer domain are crucial for VWF-cleavage. C-terminal TSP1 repeats and the CUB domains seem to be important for VWF substrate recognition and binding to potential surface receptors like CD36 on endothelial cells.

The ADAMTS13 may be a full-length, truncated, or modified isolated or recombinant ADAMTS13. In certain embodiments, recombinant ADAMTS13 (rADAMTS13) is preferred. The rADAMTS13 may correspond to or be derived from the native ADAMTS13 sequence of any mammal, including for example a rodent (e.g. a mouse) or a human. In certain embodiments, a human rADAMTS13 sequence is preferred.

In certain embodiments of the formulations provided herein, the ADAMTS13 is rADAMTS13, obtained by methods as described in the state of the art. In certain embodiments, the ADAMTS13 is a human ADAMTS13 (hA13), a recombinant human ADAMTS13 (rhA13), or a biologically active derivative or fragment thereof. In certain embodiments the amino acid sequence of hA13 is that of GenBank accession number NP 620594. In other embodiments, the amino acid sequence of hA13 comprises amino acids 75 to 1427 of NP 620594, a natural or conservative variant thereof, or a biologically active fragment thereof. These sequences may be employed in the production of recombinant human ADAMTS13 proteins.

In some embodiments, the ADAMTS13 comprises the amino acid sequence set forth in SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the nucleotide sequence that encodes the ADAMTS13 comprises the nucleotide sequence that encodes the amino acid sequence of SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the ADAMTS13 comprises the amino acid sequence set forth in SEQ ID NO: 1. In certain embodiments, the ADAMTS13 consists of the amino acid sequence set forth in SEQ ID NO: 1. In certain embodiments, the ADAMTS13 consists essentially of the amino acid sequence set forth in SEQ ID NO: 1.

In certain embodiments, the present disclosure provides variants of ADAMTS13. In certain embodiment, an ADAMTS13 variant can include one or more amino acid substitutions, deletions, insertions and/or frame shifts as compared to the amino acid sequence of a natural/wildtype ADAMTS13 (e.g., SEQ ID NO: 1) and includes naturally occurring allelic variants or alternative splice variants. For example, the ADAMTS13 variant can include at least one single amino acid substitution as compared to the wildtype ADAMTS13. The amino acid substitution(s) can be within the catalytic domain, the disintegrin domain, and/or the first thrombospondin type 1 domains (C1 and C2).

In certain embodiments, the ADAMTS13 variant comprises at least one single amino acid substitution as compared to the wildtype amino acid (e.g., SEQ ID NO: 1). In certain embodiments, the single amino acid substitution is within the catalytic domain of ADAMTS13 (e.g., amino acids 80 to 286 of SEQ ID NO: 1). In certain embodiments, the single amino acid substitution is at least one of I⁷⁹M, V⁸⁸M, H⁹⁶D, Q⁹⁷R, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13. In certain embodiments, the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13.

In certain embodiments, the ADAMTS13 variant comprises a single amino acid substitution at Q⁹⁷ as denoted in SEQ ID NO: 1, or the equivalent amino acid position in an ADAMTS13. In certain embodiments, the amino acid change is from a Q to a D, E, K, H, L, N, P, or R. In certain embodiments, the amino acid change is from a Q to an R. In certain embodiments, the ADAMTS13 variant is ADAMTS13 Q⁹⁷R (SEQ ID NO: 2, or an amino acid sequence having at least 80% sequence identity thereof while still maintaining R⁹⁷). In some embodiments, the ADAMTS13 variant comprises the amino acid sequence set forth in SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the nucleotide sequence that encodes the ADAMTS13 variant comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2, while still maintaining R⁹⁷. In certain embodiments, the ADAMTS13 variant comprises the amino acid sequence set forth in SEQ ID NO: 2. In certain embodiments, the ADAMTS13 variant consists of the amino acid sequence set forth in SEQ ID NO: 2. In certain embodiments, the ADAMTS13 variant consists essentially of the amino acid sequence set forth in SEQ ID NO: 2.

In certain embodiments, the ADAMTS13 variant is R⁷W, Q⁴⁴X, T¹⁶⁷M, Y³⁰⁴C, C³¹¹Y, T³³⁹R, P³⁴¹L, C³⁴⁷S, R³⁴⁹C, P³⁵³L, W³⁹⁰X, W³⁹⁰C, R³⁹⁸H, Q⁴⁴⁸E, Q⁴⁴⁹X, Q⁴⁵⁶H, P⁴⁵⁷L, P⁴⁷⁵S, R⁵⁰⁷Q, C⁵⁰⁸Y, G⁵²⁵D, R⁵²⁸G, A⁵⁹⁶V, A⁶⁰⁶P, P⁶¹⁸A, R⁶²⁵H, P⁶⁷¹L, I⁶⁷³F, R⁶⁹²C, Q⁷²³K, A⁷³²V, E⁷⁴⁰K, C⁷⁵⁸R, V⁸³²M, A⁹⁰⁰V, S⁹⁰³L, C⁹⁰⁸S, C⁹⁰⁸Y, R⁹¹⁰X, Q⁹²⁹X, C⁹⁵¹G, G⁹⁸²R, A¹⁰³³T, W¹⁰¹⁶X, c¹⁰²⁴G, A¹⁰³³T, R¹⁰³⁴X, S¹⁰³⁶X, R¹⁰⁶⁰W, R¹¹²³C, R¹¹⁴⁹W, R¹²⁰⁶X, C¹²¹³Y, I¹²¹⁷T, R¹²¹⁹W, T¹²²⁶I, G¹²³⁹V, W¹²⁴⁵X, Q¹³⁰²X, S¹³¹⁴L, and/or R¹³³⁶W, or the equivalent amino acid position in an ADAMTS13. In certain embodiments, the ADAMTS13 variant is not R⁷W, Q⁴⁴X, T¹⁶⁷M, Y³⁰⁴C, C³¹¹Y, T³³⁹R, P³⁴¹L, C³⁴⁷S, R³⁴⁹C, P³⁵³L, W³⁹⁰X, W³⁹⁰C, R³⁹⁸H, Q⁴⁴⁸E, Q⁴⁴⁹X, Q⁴⁵⁶H, P⁴⁵⁷L, P⁴⁷⁵S, R⁵⁰⁷Q, C⁵⁰⁸Y, G⁵²⁵D, R⁵²⁸G, A⁵⁹⁶V, A⁶⁰⁶P, P⁶¹⁸A, R⁶²⁵H, P⁶⁷¹L, I⁶⁷³F, R⁶⁹²C, Q⁷²³K, A⁷³²V, E⁷⁴⁰K, C⁷⁵⁸R, V⁸³²M, A⁹⁰⁰V, S⁹⁰³L, C⁹⁰⁸S, C⁹⁰⁸Y, R⁹¹⁰X, Q⁹²⁹X, C⁹⁵¹G, G⁹⁸²R, A¹⁰³³T, W¹⁰¹⁶X, c¹⁰²⁴G, A¹⁰³³T, R¹⁰³⁴X, S¹⁰³⁶X, R¹⁰⁶⁰W, R¹¹²³C, R¹¹⁴⁹W, R¹²⁰⁶X, C¹²¹³Y, I¹²¹⁷T, R¹²¹⁹W, T¹²²⁶I, G¹²³⁹V, W¹²⁴⁵X, Q¹³⁰²X, S¹³¹⁴L, and/or R¹³³⁶W as denoted in SEQ ID NO: 1, or the equivalent amino acid position in an ADAMTS13.

In certain embodiments, the ADAMTS13 variants provided herein retain significant ADAMTS13 activity. In certain embodiments, the ADAMTS13 variants provided equal ADAMTS13 activity as a wildtype ADAMTS13. In certain embodiments, the ADAMTS13 variants provided greater ADAMTS13 activity than a wildtype ADAMTS13 on its own.

In certain embodiments, the present invention provides compositions of ADAMTS13 variant(s), such as compositions with the constituents described in U.S. Patent Application Publication No. 2011/0229455 and/or in U.S. Patent Application Publication No. 2014/0271611, each of which are incorporated herein by reference in their entirety and for all purposes. In other aspects, the present invention provides compositions of ADAMTS13 variant(s) in combination with plasma derived ADAMTS13 and/or recombinant ADAMTS13 (rADAMTS13) proteins. In certain embodiments, the ADAMTS13 is human ADAMTS13 or a biologically active derivative or fragment thereof as described in U.S. Patent Application Publication No. 2011/0229455 and/or in U.S. Patent Application Publication No. 2014/0271611, each of which are incorporated herein by reference in their entirety and for all purposes. In one embodiment, the amino acid sequence of hA13 is that of GenBank accession number NP_620594. In certain embodiments, the amino acid sequence of hADAMTS13 is SEQ ID NO: 1 or an amino acid sequence having at least 80% sequence identity thereof. In another embodiment, the amino acid sequence of hA13 comprises amino acids 75 to 1427 of NP_620594, a natural or conservative variant thereof, or a biologically active fragment thereof. In certain embodiments, the ADAMTS13 variant is ADAMTS13 Q⁹⁷R (SEQ ID NO: 2) or an amino acid sequence having at least 80% sequence identity thereof, while still maintaining R⁹⁷.

In certain embodiments, the pharmaceutical composition comprises a combination of at least one ADAMTS13 variant and ADAMTS13 protein (e.g., wildtype). In certain embodiments, the relative abundance (e.g., percentage) of ADAMTS13 variant present in the total amount of ADAMTS13 in the composition (i.e., including all ADAMTS13 variant(s) and wildtype) is between about 5% to about 95%, about 10% to about 90%, about 15% to about 85%, about 20% to about 80%, about 25% to about 75%, about 30% to about 70%, about 35% to about 65%, about 40% to about 60%, to about 45% to about 55%. In certain embodiments, the percentage of ADAMTS13 variant present in the total amount of ADAMTS13 in the composition is between about 40% to about 90%, about 40% to about 80%, about 45% to about 75%, about 50% to about 80%, about 50% to about 70%, or about 55% to about 65%. In certain embodiments, the percentage of ADAMTS13 variant present in the total amount of ADAMTS13 in the composition is between about 50% to about 75%, about 52% to about 72%, about 55% to about 70%, about 59% to about 72%. In certain embodiments, the percentage of ADAMTS13 variant present in the total amount of ADAMTS13 in the composition is between about 45% to about 85% or about 47% to about 84%. In certain embodiments, the percentage of ADAMTS13 variant present in the total amount of ADAMTS13 in the composition is between about 47% to about 84%. In certain embodiments, the percentage of ADAMTS13 variant present in the total amount of ADAMTS13 in the composition is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In certain embodiments, the percentage of ADAMTS13 variant present in the total amount of ADAMTS13 in the composition is about 52%, about 60%, about 61%, about 62%, about 63%, about 64%, about 65%, about 66%, or about 72%. In certain embodiments, the percentage of ADAMTS13 variant present in the total amount of ADAMTS13 in the composition is about 52%, about 65%, or about 72%.

In certain embodiments, the pharmaceutical composition comprises a combination of at least one ADAMTS13 variant and ADAMTS13 protein (e.g., wildtype). In certain embodiments, the ratio of ADAMTS13 variant to ADAMTS13 protein is about 4:1 to about 1:4, about 3:1 to about 1:3, about 2:1 to about 1:2. In certain embodiments, the ratio of ADAMTS13 variant to ADAMTS13 protein is about 3:1 to about 1:3, about 2:1 to about 1:2, or about 2:1 to about 1:3, or about 1:1 to about 1:3, or about 1:1.1 to about 1:2.9, or about 1:1.2 to about 1:2.8, or about 1:1.3 to about 1:2.7, or about 1:1.4 to about 1:2.6, or about 1:1.5 to about 1:2.5, or about 1:1.6 to about 1:2.4, or about 1:1.7 to about 1:2.3, or about 1:1.8 to about 1:2.2, or about 1:1.9 to about 1:2.1. In certain embodiments, the ratio of ADAMTS13 variant to ADAMTS13 protein is about 1.1:1 to about 2.9:1, or about 1.2:1 to about 2.8:1, or about 1.3:1 to about 2.7:1, or about 1.4:1 to about 2.6:1, or about 1.5:1 to about 2.5:1, or about 1.6:1 to about 2.4:1, or about 1.7:1 to about 2.3:1, or about 1.8:1 to about 2.2:1, or about 1.9:1 to about 2.1:1. In certain embodiments, the ratio of ADAMTS13 variant to ADAMTS13 wildtype is about 1:1 to about 1:3. In certain embodiments, the ratio of ADAMTS13 variant to ADAMTS13 wildtype is about 3:1 to about 1:1. In certain embodiments, the ratio of ADAMTS13 variant to ADAMTS13 wildtype is about 1:1.1 to about 1:2.5. In certain embodiments, the ratio of ADAMTS13 variant to ADAMTS13 wildtype is about 4:1, about 4:1.5, about 4:2, about 4:2.5, about 4:3, about 4:3.5, about 3:1, about 3:1.5, about 3:2, about 3:2.5, about 2:1, or about 2:1.5. In certain embodiments, the ratio of ADAMTS13 variant to ADAMTS13 wildtype is about 1:1.5, about 1:2, about 1:2.5, about 1:3, about 1:3.5, about 1:4, about 2:2.5, about 2:3, about 2:3.5, about 2:4, about 3:3.5, or about 3:4. In certain embodiments, the ratio of ADAMTS13 variant to ADAMTS13 wildtype is about 1:3. In certain embodiments, the ratio of ADAMTS13 variant to ADAMTS13 wildtype is about 3:1. In certain embodiments, the ratio of ADAMTS13 variant to ADAMTS13 wildtype is about 2:1. In certain embodiments, the ratio of ADAMTS13 variant to ADAMTS13 wildtype is about 1:2. In certain embodiments, the ratio of ADAMTS13 variant to ADAMTS13 wildtype is about 1:1. In certain embodiments, the ratio of ADAMTS13 variant to ADAMTS13 wildtype is about 3:2. In certain embodiments, the ADAMTS13 variant comprises a single amino acid substitution at Q97 as denoted in SEQ ID NO: 1, or the equivalent amino acid in an ADAMTS13. In certain embodiments, the ADAMTS13 variant is ADAMTS13 Q97R (SEQ ID NO: 2). In certain embodiments, the wildtype ADAMTS13 is human ADAMTS13 or a biologically active derivative or fragment thereof as described in U.S. Patent Application Publication No. 2011/0229455, which is incorporated herein by reference for all purposes. In one embodiment, the amino acid sequence of hADAMTS13 is that of GenBank accession number NP_620594. In certain embodiments, the hADAMTS13 is SEQ ID NO: 1.

In exemplary embodiments, the isolated or recombinant ADAMTS13 protein or derivative is glycosylated. In certain embodiments, the ADAMTS13 protein or derivative has a plasma half-life of at least one hour, e.g., 2, 3, 4, 5, 6, or more hours.

The rADAMTS13 can be produced by expression in a suitable prokaryotic or eukaryotic host system characterized by producing a pharmacologically effective ADAMTS13 molecule. Examples of eukaryotic cells are mammalian cells, such as CHO, COS, HEK 293, BHK, SK-Hep, and HepG2. Glycosylation may correspond, for example, to patterns produced by the host cell. In some embodiments, HEK293 cells are preferred. In other embodiments, CHO cells are preferred. There is no particular limitation to the reagents or conditions used for producing, isolating, or purifying ADAMTS13 according to the present invention and any system known in the art or commercially available can be employed.

A wide variety of vectors can be used for the preparation of the rADAMTS13 and can be selected from eukaryotic and prokaryotic expression vectors. Examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, AcS, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

In certain embodiments, an ADAMTS13 used in the formulations provided herein may be expressed, produced, or purified according to a method disclosed previously, for example, in U.S. Pat. No. 6,926,894, US 2005/0266528 (now U.S. Pat. No. 7,501,117), US 2007/0015703, U.S. patent application Ser. No. 12/437,384, U.S. patent application Ser. No. 12/847,999 (now U.S. Pat. No. 8,313,926), WO 2002/42441, U.S. Pat. Nos. 9,458,222 and 10,238,720, each of which is incorporated by reference.

In certain embodiments, the concentration of a relatively pure ADAMTS13 formulation may be determined by spectroscopy (i.e., total protein measured at A280) or other bulk determination (e.g., Bradford assay, silver stain, weight of a lyophilized powder, etc.). In other embodiments, the concentration of ADAMTS 13 may be determined by an ADAMTS13 ELISA assay (e.g., mg/mL antigen).

B. Compositions and Formulations

Provided herein are pharmaceutical compositions useful for treating a COVID-19 subject, for example to treat abnormal high levels of VWF and/or complications arising therefrom, such as deep vein thrombosis or pulmonary embolism. Such compositions comprise an effective amount of ADAMTS13 or its biologically active derivatives or variants.

The pharmaceutical composition can comprise one or more pharmaceutically acceptable carriers and/or diluents. The pharmaceutical composition can also comprise one or more additional active ingredients such as agents that stimulate ADAMTS13 production or secretion by the treated patient/subject, agents that inhibit the degradation of ADAMTS13 and thus prolong its half-life (or alternatively glycosylated variants of ADAMTS13), agents that enhance ADAMTS13 activity (for example by binding to ADAMTS13, thereby inducing an activating conformational change), or agents that inhibit ADAMTS13 clearance from circulation, thereby increasing its plasma concentration.

Formulation of the composition or pharmaceutical composition will vary according to the route of administration selected (e.g., solution or emulsion). An appropriate composition comprising the composition to be administered is prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles, in certain embodiments, include sodium chloride solution, Ringer's dextrose, dextrose and sodium chloride, lactated Ringer's or fixed oils. Intravenous vehicles, in certain aspects, include various additives, preservatives, or fluid, nutrient or electrolyte replenishers.

Compositions or pharmaceutical compositions useful in the compounds and methods of the disclosure containing at least one ADAMTS13 protein (e.g., wildtype and/or variant) as an active ingredient contain, in various aspects, pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form.

A variety of aqueous carriers, e.g., water, buffered water, 0.4% saline, 0.3% glycine, or aqueous suspensions contain, in various aspects, the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents, in some instances, are a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions, in certain embodiments, contain one or more preservatives, for example ethyl, or n-propyl, p-hydroxybenzoate.

In some certain embodiments, ADAMTS13 compositions are lyophilized for storage, and reconstituted in a suitable carrier prior to use. Any suitable lyophilization and reconstitution techniques known in the art are employed. It is appreciated by those skilled in the art that lyophilization and reconstitution leads to varying degrees of protein activity loss and that use levels are often adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In certain embodiments, the ADAMTS13 compositions, provided herein may further comprise one or more pharmaceutically acceptable excipients, carriers, and/or diluents as described in U.S. Patent Application No. 20110229455 and/or in U.S. Patent Application Publication No. 2014/0271611, each of which are incorporated herein by reference in their entirety for all purposes.

In certain embodiments, the ADAMTS13 compositions provided herein will have a tonicity in a range as described in U.S. Patent Application Publication No. 2011/0229455 and/or in U.S. Patent Application Publication No. 2014/0271611, each of which are incorporated herein by reference in their entirety for all purposes.

In some embodiments, ADAMTS13 or its biologically active variants are administered with one or more additional active ingredients such as agents that stimulate ADAMTS13 production or secretion by the treated patient/subject, agents that inhibit the degradation of ADAMTS13 and thus prolong its half-life, agents that enhance ADAMTS13 activity (for example, by binding to ADAMTS13, thereby inducing an activating conformational change), or agents that inhibit ADAMTS13 clearance from circulation, thereby increasing its plasma concentration. Another ingredient that can be co-administered include blood thinners (e.g., aspirin), anti-platelet agents, and tissue plasminogen activator (tPA), a thrombolytic serine protease that activates plasmin to cleave fibrin.

In one aspect, the present invention provides stabilized formulations of ADAMTS13 (A13) and rADAMTS13 (rA13) proteins. In one embodiment, the formulations of the invention are stable when stored at temperatures up to at least about 40° C. for at least about 6 months. In other embodiments, the formulations provided herein retain significant ADAMTS13 activity when stored for extended periods of time. In yet other embodiments, the formulations of the invention reduce or retard dimerization, oligomerization, and/or aggregation of an ADAMTS13 protein. In one embodiment, the present invention provides formulations of ADAMTS13 comprising a therapeutically effective amount or dose of an ADAMTS13 protein, a sub-physiological to physiological concentration of a pharmaceutically acceptable salt, a stabilizing concentration of one or more sugars and/or sugar alcohols, a non-ionic surfactant, a buffering agent providing a neutral pH to the formulation, and optionally a calcium and/or zinc salt. Generally, the stabilized ADAMTS13 formulations provided herein are suitable for pharmaceutical administration. In a preferred embodiment, the ADAMTS13 protein is human ADAMTS13 or a biologically active derivative or fragment thereof.

In one embodiment, the present invention provides a method comprising administering to a subject in need thereof an ADAMTS13 formulation comprising (a) at least 100 units ADAMTS13 activity (i.e., FRETS-vWF73 activity) per mg ADAMTS13; (b) 0 mM to 200 mM or 0 mM to 100 mM of a pharmaceutically acceptable salt; (c) 0.5 mM to 20 mM calcium; (d) a sugar and/or sugar alcohol; (e) a nonionic surfactant; and (f) a buffering agent for maintaining a pH between 6.0 and 8.0. In one embodiment, the stabilized formulation of ADAMTS13 comprises at least 200 units A13 activity per mg ADAMTS13. In another embodiment, the stabilized formulation of ADAMTS13 comprises at least 400 units A13 activity per mg ADAMTS13. In a preferred embodiment, the stabilized formulation of ADAMTS13 comprises at least 600 units A13 activity per mg ADAMTS13. In a more preferred embodiment, the stabilized formulation of ADAMTS13 comprises at least 800 units A13 activity per mg ADAMTS13. In yet another preferred embodiment, the stabilized formulation of ADAMTS13 comprises at least 1000 units A13 activity per mg ADAMTS13. In one embodiment, the stabilized formulation of ADAMTS13 comprises between about 100 units and about 2000 units of ADAMTS13 activity per mg ADAMTS13. In a specific embodiment, the present invention provides a stabilized formulation of ADAMTS13 (A13) comprising (a) at least 100 units ADAMTS13 activity per mg ADAMTS13; (b) 0 to 200 mM or 0 mM to 100 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In certain embodiments, the composition comprises at least about 200 units, about 200 to about 400 units, about 200 to about 300 units, about 300 units, or about 294 units ADAMTS13 activity per mg or per ml.

C. Methods of Treatment

In certain aspects, the invention provides a method of treating or preventing at least one condition or complication in a subject infected with SARS-CoV-2 or suffering from COVID-19, comprising administering a composition comprising isolated or recombinant ADAMTS13. In certain embodiments, the subject is administered the composition comprising isolated or recombinant ADAMTS13 before the condition or complication is present. In certain embodiments, the subject is administered the composition comprising isolated or recombinant ADAMTS13 after the condition or complication is present.

In certain aspects, the invention provides a method of treating a subject at risk of developing at least one condition or complication associated with SARS-CoV-2 infection or COVID-19, comprising administering a composition comprising isolated or recombinant ADAMTS13. In certain embodiments, the subject is administered the composition comprising isolated or recombinant ADAMTS13 before the condition or complication is present. In certain embodiments, the subject is administered the composition comprising isolated or recombinant ADAMTS13 after the condition or complication is present.

Provided herein are pharmaceutical compositions and methods useful for treating a COVID-19 subject, for example to treat abnormally high levels of VWF and/or complications or risk factors arising from COVID-19, as described herein, and especially deep vein thrombosis or pulmonary embolism. Such compositions comprise an effective amount of isolated or recombinant ADAMTS13 or its biologically active derivatives or variants thereof. In certain embodiments, the compositions comprise rADAMTS13. In certain embodiments, the compositions comprise hrADAMTS13 (e.g., at least 80%, 85%, 90%, 91%, 92%, 93%, 94%, 95%, 96%, 97%, 98%, 99%, or 100% identity, to a reference sequence, e.g., SEQ ID NO:1). In certain embodiments, the compositions comprise a variant of ADAMTS13 (e.g., at least 80%, 85%, 90%, 91%, 92%, 93%, 94%, 95%, 96%, 97%, 98%, 99%, or 100% identity, to a reference sequence, e.g., SEQ ID NO:2).

The ADAMTS13 can be administered to mammals, particularly humans, for prophylactic and/or therapeutic purposes. In some embodiments, the invention is used to reduce the harmful effects of a coronavirus-related blood clotting disorder, blood vessel occlusion, or infarction, e.g. DVT or pulmonary embolism, without increasing the likelihood of hemorrhage or disabling the peripheral immune system. In some embodiments, ADAMTS13 is administered prophylactically, e.g., to an subject at risk of a coronavirus-related blood clotting disorder or blood vessel occlusion. Examples of subjects that can be treated according to the invention include those that have experienced or are experiencing such a disorder, thrombosis or embolism. This is especially true if the condition is severe, or if the ADAMTS13 can be administered soon after a COVID-19 diagnosis and a diagnosis of any relevant disorder, history, or risk factor.

ADAMTS13 contributes to the regulation and breakdown of von Willebrand Factor (VWF). The VWF protein is a large multimeric glycoprotein that is present in blood plasma and plays a major role in blood coagulation. VWF is stored in an ultra large form 5 (UL-VWF, >20 million Da) in platelet a-granules and Weibel-Palade bodies of endothelial cells from which it is released during injury or inflammation. If not immediately consumed for platelet adhesion, the UL-VWF is cleaved by ADAMTS13 to smaller less adhesive multimers that circulate in plasma. Ischemia, such as occurs after thrombolysis, is a potent inducer of Weibel-Palade body secretion, thus making the infarct area highly thrombogenic. The basic VWF monomer is a 2050-amino acid protein that includes a number of specific domains with a specific function: (1) the D′/D3 domain, which binds to Factor VIII; (2) the A1 domain, which binds to platelet GPlb-receptor, heparin, and possibly collagen; (3) the A3 domain, which binds to collagen; (4) the Cl domain, in which the R-G-D motif binds to platelet integrin αIIbβ3 when this is activated; and (5) the “cysteine knot” domain located at the C terminus, which VWF shares with platelet-derived growth factor (PDGF), transforming growth factor-β(TGFβ), and β-human chorionic gonadotropin (βHCG). Multimers of VWF can be extremely large, consisting of over 80 monomers with molecular weight exceeding 20,000 kDa. These large VWF multimers are most biologically functional, capable of mediating the adhesion of platelets to sites of vascular injury, as well as binding and stabilizing the procoagulant protein Factor VIII. Deficiency in VWF or altered VWF is known to cause various bleeding disorders.

According to the invention, administration of isolated or recombinant ADAMTS13, preferably rADAMTS13, will treat earlier stage and later stage COVID-19 subjects, particularly subjects who present “at risk,” because of age (e.g. at least about 65 years of age) and/or because of one or more complications or risk factors associated with COVID-19. These include, for example and not for limitation, elevated levels of VWF and/or its multimers (especially ultralarge multimers (UHMW)), elevated activity levels of VWF, reduced plasma levels of endogenous ADAMTS13, reduced activity of endogenous ADAMTS13, elevated cytokine levels, coagulopathies, blood-clotting disorders, veno-occlusive disorders, prothrombotic conditions, inherited thrombotic thrombocytopenic purpura (TTP), acquired TTP, disseminated intravascular coagulation (DIC), sepsis, sickle cell, renal failure, respiratory failure, acute respiratory distress syndrome (ARDS), chronic obstructive pulmonary disease (COPD), pneumonia, asthma, pregnancy, menopause, peri-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof (collectively, “risk factors” or “complications”).

Without wishing to be bound by any theory, ADAMTS13 will be safe and effective for “at risk” COVID-19 subjects, including treatment or prophylaxis for earlier stage patients, and treatment or rescue therapy for later stage and critically ill patients, for at least the following reasons: (1) ADAMTS13 is capable of rapidly cleaving VWF, with a reduction of the molecular size of VWF (notably, a loss of ultralarge multimers); (2) ADAMTS13-mediated cleavage of VWF will reduce the adhesivity of VWF to platelet and vascular proteins thus reducing the formation of platelet thrombi; (3) occurrence of supra-physiological levels of VWF could consume ADAMTS13 by substrate overload and lower active levels of circulatory ADAMTS13 below an effective or critical threshold; (4) ADAMTS13 is a highly selective proteolytic enzyme, its only known function is cleavage of VWF. VWF and ADAMTS13 do not consume each other and do not form a precipitate when bound to each other. In vivo, it is believed that sheer forces are required to elongate part of the VWF protein structure, so that it is accessible to VWF. This provides additional regulation of VWF and ADAMTS13 equilibrium and balance, and further reduces sensitivity to overdose. ADAMTS13 is likely to be well-tolerated with few, if any, adverse side effects, including an advantageous lack of adverse hemorrhaging. Currently, COVID-19 patients are often given an anticoagulant, such as heparin, which can cause serious hemorrhaging problems.

In certain embodiments, a pharmaceutical composition comprising a therapeutically effective amount of ADAMTS13 is administered upon a finding or diagnosis of coronavirus infection, e.g., a SARS-CoV-2 infection, or a diagnosis of a coronavirus disease, e.g., COVID-19. In certain embodiments, diagnosis of infection and/or disease is based on a suitable laboratory test. In certain embodiments, the composition is administered to a coronavirus subject, e.g. a SARS-CoV-2 infected or COVID-19 patient, upon a finding or diagnosis that the subject exhibits signs or symptoms of a coagulopathy, clotting disorder, infarction, thrombosis, or embolism. In certain embodiments the thrombosis is deep vein thrombosis (DVT). In certain embodiments, the embolism is pulmonary embolism (PE).

In certain embodiments, the composition is administered to a coronavirus, SARS-CoV-2 infected, or COVID-19 subject upon a finding or diagnosis of an abnormally high level of VWF protein or VWF multimers (e.g., UHMW). This determination can be made using a suitable laboratory test, such as an ELISA assay using materials and methods described in Turecek et al., Seminars In Thrombosis and Hemostasis, 2002 Vol. 28, No. 2, 149-160; Turecek, et al., Seminars In Thrombosis and Hemostasis, 2010 Vol. 36, No. 5, 510-521, each incorporated by reference in their entirety for all intended purposes.

In certain embodiments an abnormally high level of VWF or its multimers (e.g., UHMW) is shown when levels significantly exceed, by at least about 5%, 10%, 15%, 20%, 25%, 30%, 35%, 40%, 45%, 50%, 55%, 60%, 65%, 70%, 75%, 80%, 85%, 90%, 95%, or 100% one or both of a) a normal VWF:antigen range of about 50-200% of the predetermined baseline value or about 42-136% of the predetermined baseline value or b) a normal VWF:activity of about 42-168% of the predetermined baseline value. See e.g., Swystun supra and Escher 190:62, supra, each incorporated by reference in their entirety for all intended purposes. In certain embodiments, the level of VWF or its multimers (e.g., UHMW) exceeds at least about 5%, 10%, 15%, 20%, 25%, 30%, 35%, 40%, 45%, 50%, 55%, 60%, 65%, 70%, 75%, 80%, 85%, 90%, 95%, or 100% one or both of a) a normal VWF:antigen predetermined baseline range or value or b) a normal VWF:activity predetermined baseline range or value. In certain embodiments, the level of VWF or its multimers (e.g., UHMW) exceeds at least about 5%, 10%, or 20% one or both of a) a normal VWF:antigen predetermined baseline range or value or b) a normal VWF:activity predetermined baseline range or value.

In certain embodiments, the composition is administered upon a finding or diagnosis of abnormally low levels of ADAMTS13. This determination can be made using a suitable laboratory test. Suitable methods are described in Kokame et al., Br J Haematol 2005; 129: 93-100; Tripodi, J Thromb Haemost 2004 v2 p 1601-9; Tripodi, et. al., J Thromb Haemost. 2008 September; 6(9): 1534-1541, each incorporated by reference in their entirety for all intended purposes. In certain embodiments, the normal or baseline range of ADAMTS13 levels in healthy individuals lies between 40-160% or between 87-113% of a predetermined baseline value. See e.g., Peyvandi, supra and Mancini, supra, each incorporated by reference in their entirety for all intended purposes.

In certain embodiments an abnormally low level of ADAMTS13 is shown when ADAMTS13 level and/or activity is 20%, 30%, 40%, 50%, 60%, 70%, 80%, or 90% of the normal range. In certain embodiments an abnormally low level of ADAMTS13 is shown when ADAMTS13 level and/or activity is 20%, 30%, 40%, 50%, 60%, or 70% of the normal baseline. In certain embodiments, the composition is administered within 24 hours of any one of these findings or diagnoses. In other embodiments, the composition is administered within 12 hours of any of these findings or diagnoses. In other embodiments, the composition is administered within 8, 6, 4 or 2 hours of any of these findings or diagnoses. In other embodiments, the composition is administered within one hour of any of these findings or diagnoses.

In certain embodiments, the predetermined normal baseline is based on a normal control population in the testing laboratory with the validated/chosen methods of measurement. In certain embodiments, when a baseline range is provided, the subject's sample is compared to the upper limit of the range when evaluating an increase compared to normal control. In certain embodiments, when a baseline range is provided, the subject's sample is compared to the lower limit of the range when evaluating an decrease compared to normal control. In certain embodiments, when a baseline range is provided, the subject's sample is compared to the mean, median, or mode of the predetermined baseline range. In certain embodiments, when a baseline range is provided, the subject's sample is compared to the mean of the predetermined baseline range.

The route of administration does not exhibit a specific limitation and can be, for example, subcutaneous, intraarterial, or intravenous. Oral administration of ADAMTS13 is also a possibility. In certain embodiments, intravenous administration is preferred. In certain embodiments, subcutaneous administration is preferred. For example, the rADAMTS13 composition may be administered intravenously when a suitable liquid or a liquid reconstituted from a lyophilized formulation is provided. The intravenous A13 dose for a COVID-19 may be indicated as IU/kg. The subcutaneous A13 dose for a COVID-19 may be indicated as IU/kg.

D. Methods of Determining a Subject is at Risk

In certain aspects, the invention provides a method of determining whether a subject diagnosed with COVID-19 is at an increased risk for a thrombotic coagulopathy, said method comprising the steps of: a) measuring in a blood plasma sample one or more of: i) a plasma level of VWF protein; ii) an activity level of VWF in the plasma sample; iii) a plasma level of UHMW VWF protein multimers; iv) a plasma level of ADAMTS13 protein; or v) an activity level of ADAMTS13 protein in the plasma sample; and b) comparing the plasma level(s) or activity level(s) measured in step a) to a baseline range or baseline value for the same plasma level(s) or activity level(s); and c) identifying the subject being at risk for a thrombotic coagulopathy when at least one of the following is met: i) the plasma level of VWF protein is increased; ii) the activity level of VWF is increased; iii) plasma UHMW VWF protein multimers are detected or the plasma level of UHMW VWF protein multimers is increased; iv) the plasma level of ADAMTS13 protein is decreased; or v) the activity level of ADAMTS13 protein is decreased, as compared to the baseline range or baseline value for the same plasma level(s) or activity level(s).

In certain embodiments, a thrombotic coagulopathy includes, but is not limited to, platelet aggregation, blood clotting, a thrombosis, a thrombotic microangiopathy, an embolism, an infarction, veno-occlusion, a stroke, renal failure resulting from thrombosis, or combinations thereof. In certain embodiments, the thrombosis is deep vein thrombosis (DVT). In certain embodiments, embolism is a pulmonary embolism (PE). In certain embodiments, the thrombotic coagulopathy is renal failure resulting from thrombosis.

In certain embodiments, at least the plasma level of VWF protein is increased. In certain embodiments, at least the activity level of VWF is increased. In certain embodiments, at least UHMW VWF protein multimers are detected. In certain embodiments, at least the plasma level of UHMW VWF protein multimers is increased. In certain embodiments, at least the plasma level of ADAMTS13 protein is decreased. In certain embodiments, at least the activity level of ADAMTS13 protein is decreased.

In certain embodiments, thrombotic coagulopathy includes, but is not limited to, platelet aggregation, blood clotting, a thrombosis, a thrombotic microangiopathy, an embolism, an infarction, veno-occlusion, a stroke, renal failure resulting from thrombosis, or combinations thereof. In certain embodiments, the thrombosis is deep vein thrombosis (DVT). In certain embodiments, the embolism is a pulmonary embolism (PE). In certain embodiments, the thrombotic coagulopathy is renal failure resulting from thrombosis.

In certain embodiments, the subject is at risk for developing a thrombotic coagulopathy when the plasma level of VWF protein and/or VWF activity is, separately, about 100-600%, about 100-500%, about 100-400%, about 100-300%, about 100-250%, about 100-200%, about 110-600%, about 110-500%, about 110-400%, about 110-300%, about 110-250%, about 110-200%, about 115-600%, about 115-500%, about 115-400%, about 115-300%, about 115-250%, about 115-200%, about 120-600%, about 120-500%, about 120-400%, about 120-300%, about 120-250%, or about 120-200% of the baseline value for said VWF protein plasma level and/or VWF activity. In certain embodiments, the subject is at risk for developing a thrombotic coagulopathy when the plasma level of VWF protein and/or VWF activity is, separately, about 100-400%, about 110-350%, about 120-300%, about 130-250%, or about 140-200% of the baseline value for said VWF protein plasma level and/or VWF activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of VWF protein and/or VWF activity is, separately, about 100-400%, about 100-350%, about 100-300%, about 100-250%, about 100-200%, about 100-140%, about 100-130%, about 100-120%, or about 100-110% of the baseline value for said VWF protein plasma level and/or VWF activity. In certain embodiments, the subject is at risk for developing a thrombotic coagulopathy when the plasma level of VWF protein and/or VWF activity is, separately, about 110-400%, about 110-350%, about 110-300%, about 110-250%, about 110-200%, about 110-140%, about 110-130%, or about 110-120% of the baseline value for said VWF protein plasma level and/or VWF activity. In certain embodiments, the subject is at risk for developing a thrombotic coagulopathy when the plasma level of VWF protein and/or VWF activity is, separately, about 120-400%, about 120-350%, about 120-300%, about 120-250%, about 120-200%, about 120-140%, or about 120-130% of the baseline value for said VWF protein plasma level and/or VWF activity. In certain embodiments, the subject is at risk for developing a thrombotic coagulopathy when the plasma level of VWF protein and/or VWF activity is, separately, about 130-400%, about 130-350%, about 130-300%, about 130-250%, about 130-200%, or about 130-140% of the baseline value for said VWF protein plasma level and/or VWF activity. In certain embodiments, the subject is at risk for developing a thrombotic coagulopathy when the plasma level of VWF protein and/or VWF activity is, separately, about 140-400%, about 140-350%, about 140-300%, about 140-250%, or about 140-200% of the baseline value for said VWF protein plasma level and/or VWF activity. In certain embodiments, the subject is at risk for developing a thrombotic coagulopathy when the plasma level of VWF protein and/or VWF activity is, separately, about 200-400%, about 200-350%, about 200-300%, or about 200-250% of the baseline value for said VWF protein plasma level and/or VWF activity. In certain embodiments, the subject is at risk for developing a thrombotic coagulopathy when the plasma level of VWF protein and/or VWF activity is, separately, about 250-400%, about 250-350, or about 250-300% of the baseline value for said VWF protein plasma level and/or VWF activity. In certain embodiments, the subject is at risk for developing a thrombotic coagulopathy when the plasma level of VWF protein and/or VWF activity is, separately, about 300-400%, about 300-350%, or about 350-400% of the baseline value for said VWF protein plasma level and/or VWF activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of VWF protein and/or VWF activity is, separately, about or at least about 100%, 105%, 110%, 115%, 120%, 125%, 130%, 135%, 140%, 145%, 150%, 155%, 160%, 165%, 170%, 175%, 180%, 185%, 190%, 200%, 225%, 250%, 275%, 300%, 325%, 350%, 375%, 400%, 425%, 450%, 475%, 500%, 525%, 550%, 575%, or 600% of the baseline value for said VWF protein plasma level and/or VWF activity. In certain embodiments, the subject is at risk for developing a thrombotic coagulopathy when the plasma level of VWF protein and/or VWF activity is, separately, at least about 100%, at least about 120%, at least about 200% or at least about 300% or more of the baseline value for said VWF protein plasma level and/or VWF activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of VWF protein and/or VWF activity is, separately, about 100-400%, about 110-350%, about 120-300%, about 130-250%, or about 140-200% of the baseline value for said VWF protein plasma level and/or VWF activity. In certain embodiments, the subject is at risk for developing a thrombotic coagulopathy when the plasma level of VWF protein and/or VWF activity is, separately, about 100-300%, about 110-300%, about 120-300%, about 130-300%, or about 140-300% of the baseline value for said VWF protein plasma level and/or VWF activity. In certain embodiments, the subject is at risk for developing a thrombotic coagulopathy when the plasma level of VWF protein and/or VWF activity is, separately, about 100-250%, about 110-250%, about 120-250%, about 130-250%, or about 140-250% of the baseline value for said VWF protein plasma level and/or VWF activity. In certain embodiments, the subject is at risk for developing a thrombotic coagulopathy when the plasma level of VWF protein and/or VWF activity is, separately, about 100-200%, about 110-200%, about 120-200%, about 130-200%, or about 140-200% of the baseline value for said VWF protein plasma level and/or VWF activity. In certain embodiments, the subject is at risk for developing a thrombotic coagulopathy when the plasma level of VWF protein and/or VWF activity is, separately, about 200-220%, about 220-240%, about 240-260%, about 260-280%, or about 280-300% of the baseline value for said VWF protein plasma level and/or VWF activity. In certain embodiments, the subject is at risk for developing a thrombotic coagulopathy when the plasma level of VWF protein and/or VWF activity is, separately, about 120-300% of the baseline value for said VWF protein plasma level and/or VWF activity. In certain embodiments, subjects with this risk are administered lower doses of ADAMTS13 (e.g., about 10-40 IU/kg).

In certain embodiments, the subject is at risk for developing a thrombotic coagulopathy when the plasma level of VWF protein and/or VWF activity is, separately, about or at least about 100%, 105%, 110%, 115%, 120%, 125%, 130%, 135%, 140%, 145%, 150%, 155%, 160%, 165%, 170%, 175%, 180%, 185%, 190%, 200%, 225%, 250%, 275%, 300% of the baseline value for said VWF protein plasma level and/or VWF activity. In certain embodiments, the subject is at risk for developing a thrombotic coagulopathy when the plasma level of VWF protein and/or VWF activity is, separately, about 100-120%, about 120-140%, about 140-160%, about 160-180%, about 180-200%, about 200-220%, about 220-240%, about 240-260%, about 260-280%, or about 280-300% of the baseline value for said VWF protein plasma level and/or VWF activity. In certain embodiments, subjects with this risk are administered lower doses of ADAMTS13 (e.g., about 10-40 IU/kg).

In certain embodiments, the subject is at high risk for developing a thrombotic coagulopathy when the plasma level of VWF protein and/or VWF activity is, separately, about or at least about 300%, 325%, 350%, 375%, 400%, 425%, 450%, 475%, 500%, 525%, 550%, 575%, 600% or more of the baseline value for said VWF protein plasma level and/or VWF activity. In certain embodiments, the subject is at high risk for developing a thrombotic coagulopathy when the plasma level of VWF protein and/or VWF activity is, separately, at least about 250, 275, 300, 325, or 350% of the baseline value for said VWF protein plasma level and/or VWF activity. In certain embodiments, the subject is at high risk for developing a thrombotic coagulopathy when the plasma level of VWF protein and/or VWF activity is, separately, at least about 300% of the baseline value for said VWF protein plasma level and/or VWF activity. In certain embodiments, subjects with this risk are administered higher doses of ADAMTS13 (e.g., about 40-400 IU/kg).

In certain embodiments, the subject is at risk for developing a thrombotic coagulopathy when the plasma level of ADAMTS13 protein and/or ADAMTS13 activity is, separately, about 50-100%, about 55-100%, about 60-100%, about 65-100%, about 70-100%, about 75-100%, about 80-100%, about 85-100%, or about 90-100% of the baseline value for said ADAMTS13 protein plasma level and/or ADAMTS13 activity. In certain embodiments, the subject is at risk for developing a thrombotic coagulopathy when the plasma level of ADAMTS13 protein and/or ADAMTS13 activity is about 70-100% of the baseline value for said ADAMTS13 protein plasma level and/or ADAMTS13 activity. In certain embodiments, the subject is at risk for developing a thrombotic coagulopathy when the plasma level of ADAMTS13 protein and/or ADAMTS13 activity is no more than about 60, 65, 70, or 75% of the baseline value for said ADAMTS13 protein plasma level and/or ADAMTS13 activity. In certain embodiments, the subject is at risk for developing a thrombotic coagulopathy when the plasma level of ADAMTS13 protein and/or ADAMTS13 activity is no more than about 70% of the baseline value for said ADAMTS13 protein plasma level and/or ADAMTS13 activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of ADAMTS13 protein and/or ADAMTS13 activity is, separately, about 100%, about 95%, about 90%, about 85%, about 80%, about 75%, about 70%, about 65%, about 60%, about 55%, about 50%, about 45%, about 40%, about 35%, about 30%, about 25%, or about 20% of the baseline value for said ADAMTS13 protein plasma level and/or ADAMTS13 activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of ADAMTS13 protein and/or ADAMTS13 activity is, separately, no more than about 100%, about 95%, about 90%, about 85%, about 80%, about 75%, about 70%, about 65%, about 60%, about 55%, about 50%, about 45%, about 40%, about 35%, about 30%, about 25%, about 20%, or less of the baseline value for said ADAMTS13 protein plasma level and/or ADAMTS13 activity. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the plasma level of ADAMTS13 protein and/or ADAMTS13 activity is, separately, about 65-100%, about 70-100%, about 75-100%, about 80-100%, about 85-100%, or about 90-100% of the baseline value for said ADAMTS13 protein plasma level and/or ADAMTS13 activity. In certain embodiments, the subject is at risk for developing a thrombotic coagulopathy when the plasma level of ADAMTS13 protein and/or ADAMTS13 activity is about 70-100% of the baseline value for said ADAMTS13 protein plasma level and/or ADAMTS13 activity. In certain embodiments, subjects with this risk are administered lower doses of ADAMTS13 (e.g., about 10-40 IU/kg).

In certain embodiments, the subject is at high risk for developing a thrombotic coagulopathy when the plasma level of ADAMTS13 protein and/or ADAMTS13 activity is, separately, no more than about 70%, about 65%, about 60%, about 55%, about 50%, about 45%, about 40%, about 35%, about 30%, about 25%, about 20%, or less of the baseline value for said ADAMTS13 protein plasma level and/or ADAMTS13 activity. In certain embodiments, the subject is at high risk for developing a thrombotic coagulopathy when the plasma level of ADAMTS13 protein and/or ADAMTS13 activity is no more than about 60, 65, 70, or 75% or less of the baseline value for said ADAMTS13 protein plasma level and/or ADAMTS13 activity. In certain embodiments, the subject is at high risk for developing a thrombotic coagulopathy when the plasma level of ADAMTS13 protein and/or ADAMTS13 activity is about 70% or less of the baseline value for said ADAMTS13 protein plasma level and/or ADAMTS13 activity. In certain embodiments, subjects with this risk are administered higher doses of ADAMTS13 (e.g., about 40-400 IU/kg).

In certain embodiments, the subject is at risk for developing a thrombotic coagulopathy when the plasma level of UHMW VWF multimers is about 100-200%, about 100-190%, about 100-180%, about 100-170%, about 100-160%, about 100-150%, about 100-140%, about 100-130%, about 100-120%, about 100-115%, about 100-114%, about 100-113%, about 100-112%, about 100-111%, about 100-110%, about 100-109%, about 100-108%, about 100-109%, about 100-106%, about 100-105%, about 100-104%, about 100-103%, about 100-102%, or about 100-101% of the baseline value for said UHMW VWF multimer plasma level. In certain embodiments, the subject is at risk for developing a thrombotic coagulopathy when the plasma level of UHMW VWF multimers is about 100-102%, about 100-105%, about 100-110%, about 100-115%, or about 100-120% of the baseline value for said UHMW VWF multimer plasma level. In certain embodiments, the subject is at risk for developing a thrombotic coagulopathy when the plasma level of UHMW VWF multimers is about 100-110% of the baseline value for said UHMW VWF multimer plasma level. In certain embodiments, the subject is at risk for developing a thrombotic coagulopathy when the plasma level of UHMW VWF multimers at least 110% of the baseline value for said UHMW VWF multimer plasma level. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, subject is at risk for developing a thrombotic coagulopathy when the plasma level of UHMW VWF multimers is about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 130%, about 140%, about 150%, about 160%, about 170%, about 180%, about 190%, about 200%, about 250%, about 300%, about 350%, about 400%, or more of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, subject is at risk for developing a thrombotic coagulopathy when the plasma level of UHMW VWF multimers is about 100%, about 101%, about 102%, about 103%, about 104%, about 105%, about 106%, about 107%, about 108%, about 109%, about 110%, about 111%, about 112%, about 113%, about 114%, or about 115% of the baseline value for said UHMW VWF multimer plasma level. In certain embodiments, subject is at risk for developing a thrombotic coagulopathy when the plasma level of UHMW VWF multimers is at least about 100%, about 101%, about 102%, about 103%, about 104%, about 105%, about 106%, about 107%, about 108%, about 109%, about 110%, about 111%, about 112%, about 113%, about 114%, or about 115% of the baseline value for said UHMW VWF multimer plasma level. In certain embodiments, the subject is at risk for developing a thrombotic coagulopathy when the plasma level of UHMW VWF multimers is about 100-120%, about 100-115%, about 100-114%, about 100-113%, about 100-112%, about 100-111%, about 100-110%, about 100-109%, about 100-108%, about 100-109%, about 100-106%, about 100-105%, about 100-104%, about 100-103%, about 100-102%, or about 100-101% of the baseline value for said UHMW VWF multimer plasma level. In certain embodiments, the subject is at risk for developing a thrombotic coagulopathy when the plasma level of UHMW VWF multimers is about 100-102%, about 100-105%, about 100-110%, or about 100-115% of the baseline value for said UHMW VWF multimer plasma level. In certain embodiments, the subject is at risk for developing a thrombotic coagulopathy when the plasma level of UHMW VWF multimers is about 100-110% of the baseline value for said UHMW VWF multimer plasma level. In certain embodiments, subjects with this risk are administered lower doses of ADAMTS13 (e.g., about 10-40 IU/kg).

In certain embodiments, subject is at high risk for developing a thrombotic coagulopathy when the plasma level of UHMW VWF multimers is at least about 110%, about 111%, about 112%, about 113%, about 114%, about 115%, about 116%, about 117%, about 118%, about 119%, about 120%, about 130%, about 140%, about 150%, about 160%, about 170%, about 180%, about 190%, about 200%, about 250%, about 300%, about 350%, about 400%, or more of the baseline value for said UHMW VWF multimer plasma level. In certain embodiments, subject is at risk for developing a thrombotic coagulopathy when the plasma level of UHMW VWF multimers is about 105, 110, or 115% or more of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, subjects with this risk are administered higher doses of ADAMTS13 (e.g., about 40-400 IU/kg).

In certain embodiments, the subject is at risk for developing a thrombotic coagulopathy when the ratio of VWF to ADAMTS13 (VWF:A13) in the plasma sample is about 5.0, 4.75, 4.5, 4.25, 4.0, 3.75, 3.5, 3.25, 3.0, 2.75, 2.5, 2.25, 2.0, 1.75, 1.5, 1.25, 1.0 or less. In certain embodiments, the subject is at risk for developing a thrombotic coagulopathy when the ratio of VWF to ADAMTS13 (VWF:A13) in the plasma sample is about 2.0, 3.0, or 4.0 or less. In certain embodiments, the subject is at risk for developing a thrombotic coagulopathy when the ratio of VWF to ADAMTS13 (VWF:A13) in the plasma sample is about 3.0 or less. In certain embodiments, the subject is at risk for developing a thrombotic coagulopathy when the ratio of VWF to ADAMTS13 (VWF:A13) in the plasma sample is greater than about 2.0, 3.0, or 4.0. In certain embodiments, the subject is at risk for developing a thrombotic coagulopathy when the ratio of VWF to ADAMTS13 (VWF:A13) in the plasma sample is greater than about 3.0. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the subject is at risk for developing a thrombotic coagulopathy when the ratio of VWF to ADAMTS13 (VWF:A13) in the plasma sample is less than or equal to about 3.5, 3.25, 3.0, 2.75, 2.5, 2.25, 2.0, 1.75, 1.5, 1.25, 1.0 or less. In certain embodiments, the subject is at risk for developing a thrombotic coagulopathy when the ratio of VWF to ADAMTS13 (VWF:A13) in the plasma sample is about 3.0 or less, about 2.0 or less, or about 1.0 or less. In certain embodiments, the subject is at risk for developing a thrombotic coagulopathy when the ratio of VWF to ADAMTS13 (VWF:A13) in the plasma sample is about 3.0 or less. In certain embodiments, subjects with this risk are administered lower doses of ADAMTS13 (e.g., about 10-40 IU/kg).

In certain embodiments, the subject is at high risk for developing a thrombotic coagulopathy when the ratio of VWF:A13 levels in the plasma sample is greater than about 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, or 10.0. In certain embodiments, the subject is at high risk for developing a thrombotic coagulopathy when the ratio of VWF:A13 levels in the plasma sample is greater than about 4.0. In certain embodiments, the subject is at high risk for developing a thrombotic coagulopathy when the ratio of VWF:A13 levels in the plasma sample is greater than about 3.0. In certain embodiments, subjects with this risk are administered higher doses of ADAMTS13 (e.g., about 40-400 IU/kg).

In certain embodiments, the baseline value is a predetermine value based on a normal control population. In certain embodiments, the baseline value is a mean of a predetermine range of a normal control population.

In certain embodiments, the VWF activity level is measured by VWF ristocetin co-factor activity. In certain embodiments, the VWF activity level is measured by VWF collagen binding activity. In certain embodiments, the ADAMTS13 activity level is measured by ELISA. In certain embodiments, the VWF activity level is measured by FRETS.

In certain aspects, the invention provides a method of determining whether a subject diagnosed with COVID-19 is at risk for a thrombotic coagulopathy, the method comprising the steps of: a) measuring in a blood plasma sample one or more of: i) a plasma level of VWF protein; ii) an activity level of VWF in the plasma sample; iii) a plasma level of UHMW VWF protein multimers; iv) a plasma level of ADAMTS13 protein; and/or v) an activity level of ADAMTS13 protein in the plasma sample.

In certain embodiments, the method comprises identifying the subject being at risk for a thrombotic coagulopathy when the plasma level of VWF protein is at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 6.0, 7.0, 8.0, 9.0, or 10.0 IU/ml. In certain embodiments, the method comprises identifying the subject being at risk for a thrombotic coagulopathy when the plasma level of VWF protein is at least about 1.2, 1.4, 1.6, 1.8, 2.0, 2.2, 2.4, 2.6, 2.8, 3.0, 3.2, 3.4, 3.6, 3.8, 4.0, 6.0, 8.0, 9.0, or 10.0 IU/ml. In certain embodiments, the method comprises identifying the subject being at risk for a thrombotic coagulopathy when the plasma level of VWF protein is at least about 1.2, 4.0, 4.5, 6.0, 10.0, or 10.3 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the plasma level of VWF protein is about 1.0 IU/ml to about 1.2 IU/ml, about 1.0 IU/ml to about 2.0 IU/ml, about 1.0 IU/ml to about 4.0 IU/ml, about 1.0 IU/ml to about 4.5 IU/ml, about 1.0 IU/ml to about 10.3 IU/ml, about 1.0 IU/ml to about 10.0 IU/ml, about 1.2 IU/ml to about 2.0 IU/ml, about 1.2 IU/ml to about 4.0 IU/ml, about 1.2 IU/ml to about 4.5 IU/ml, about 1.2 IU/ml to about 10.3 IU/ml, about 1.2 IU/ml to about 10.0 IU/ml, about 1.4 IU/ml to about 2.0 IU/ml, about 1.4 IU/ml to about 4.0 IU/ml, about 1.4 IU/ml to about 4.5 IU/ml, about 1.4 to about 10.3 IU/ml, or about 1.4 IU/ml to about 10 IU/ml, about 1.6 IU/ml to about 2.0 IU/ml, about 1.6 IU/ml to about 4.0 IU/ml, about 1.6 IU/ml to about 4.5 IU/ml, about 1.6 to about 10.3 IU/ml, or about 1.6 IU/ml to about 10 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the plasma level of VWF protein is at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 IU/ml. In certain embodiments, the method comprises identifying the subject being at risk for a thrombotic coagulopathy when the plasma level of VWF protein is at least about 1.2, 1.4, 1.6, 1.8, 2.0, 2.2, 2.4, 2.6, 2.8, 3.0, 3.2, 3.4, 3.6, 3.8, 4.0, 4.2, or 4.4 IU/ml. In certain embodiments, the method comprises identifying the subject being at risk for a thrombotic coagulopathy when the plasma level of VWF protein is about 1.0 IU/ml to about 5.5 IU/ml, about 1.0 IU/ml to about 5.0 IU/ml, about 1.0 IU/ml to about 4.5 IU/ml, about 1.0 IU/ml to about 4.0 IU/ml, or about 1.0 IU/ml to about 3.5 IU/ml. In certain embodiments, the method comprises identifying the subject being at risk for a thrombotic coagulopathy when the plasma level of VWF protein is about 1.1 IU/ml to about 5.5 IU/ml, about 1.1 IU/ml to about 5.0 IU/ml, about 1.1 IU/ml to about 4.5 IU/ml, about 1.1 IU/ml to about 4.0 IU/ml, or about 1.1 IU/ml to about 3.5 IU/ml. In certain embodiments, the method comprises identifying the subject being at risk for a thrombotic coagulopathy when the plasma level of VWF protein is about 1.2 IU/ml to about 5.5 IU/ml, about 1.2 IU/ml to about 5.0 IU/ml, about 1.2 IU/ml to about 4.5 IU/ml, about 1.2 IU/ml to about 4.0 IU/ml, or about 1.2 IU/ml to about 3.5 IU/ml. In certain embodiments, the method comprises identifying the subject being at risk for a thrombotic coagulopathy when the plasma level of VWF protein is about 1.3 IU/ml to about 5.5 IU/ml, about 1.3 IU/ml to about 5.0 IU/ml, about 1.3 IU/ml to about 4.5 IU/ml, about 1.3 IU/ml to about 4.0 IU/ml, or about 1.3 IU/ml to about 3.5 IU/ml. In certain embodiments, the method comprises identifying the subject being at risk for a thrombotic coagulopathy when the plasma level of VWF protein is about 1.4 IU/ml to about 5.5 IU/ml, about 1.4 IU/ml to about 5.0 IU/ml, about 1.4 IU/ml to about 4.5 IU/ml, about 1.4 IU/ml to about 4.0 IU/ml, or about 1.4 IU/ml to about 3.5 IU/ml. In certain embodiments, the method comprises identifying the subject being at risk for a thrombotic coagulopathy when the plasma level of VWF protein is at least about 1.0, 1.1, 1.2, 1.3, 1.4, 1.5, 1.6, 1.7, or 1.8 IU/ml. In certain embodiments, the method comprises identifying the subject being at risk for a thrombotic coagulopathy when the plasma level of VWF protein is at least about 1.2 IU/ml. In certain embodiments, subjects with this risk are administered lower doses of ADAMTS13 (e.g., about 10-40 IU/kg).

In certain embodiments, the subject is at a high risk for a thrombotic coagulopathy when the plasma level of VWF protein is at least about 2.0, 2.5, 3.0, 3.5, 4.0, 4.5, 5.0, 5.5, 6.0, 6.5, 7.0, 7.5, 8.0, 8.5, 9.0, 9.5, or 10.0 IU/ml. In certain embodiments, the subject is at a high risk for a thrombotic coagulopathy when the plasma level of VWF protein is at least about 4.0, 4.1, 4.2, 4.3, 4.4, 4.5, 4.6, 4.7, 4.8, 4.9, or 5.0 IU/ml. In certain embodiments, the subject is at a high risk for a thrombotic coagulopathy when the plasma level of VWF protein is at least about 3.5, 4.0, 4.5, or 5.0 IU/ml. In certain embodiments, the subject is at a high risk for a thrombotic coagulopathy when the plasma level of VWF protein is at least about 4.5 IU/ml. In certain embodiments, subjects with this risk are administered higher doses of ADAMTS13 (e.g., about 40-400 IU/kg).

In certain embodiments, the method comprises identifying the subject being at high risk for a thrombotic coagulopathy when the plasma level of VWF protein is at least about 4.0, 5.0, 6.0, 7.0, 8.0, 9.0, or 10.0 IU/ml. In certain embodiments, the method comprises identifying the subject being at high risk for a thrombotic coagulopathy when the plasma level of VWF protein is at least about 4.0, 6.0, 8.0, 9.0, or 10.0 IU/ml. In certain embodiments, the method comprises identifying the subject being at high risk for a thrombotic coagulopathy when the plasma level of VWF protein is at least about 4.0, 4.5, 6.0, 10.0, or 10.3 IU/ml. In certain embodiments, the method comprises identifying the subject being at high risk for a thrombotic coagulopathy when the plasma level of VWF protein is at least about 4.0, 4.5, or 5.0 IU/ml. In certain embodiments, the method comprises identifying the subject being at high risk for a thrombotic coagulopathy when the plasma level of VWF protein is at least about 4.5 IU/ml. In certain embodiments, the method comprises identifying the subject being at high risk for a thrombotic coagulopathy when the plasma level of VWF protein is at least about 10.0 IU/ml. In certain embodiments, the method comprises identifying the subject being at high risk for a thrombotic coagulopathy when the plasma level of VWF protein is at least about 10.3 IU/ml. In certain embodiments, subjects with this risk are administered higher doses of ADAMTS13 (e.g., about 40-400 IU/kg).

In certain embodiments, the method comprises identifying the subject being at risk for a thrombotic coagulopathy when the VWF activity level is at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 6.0, 7.0, 8.0, 9.0, or 10.0 IU/ml. In certain embodiments, the method comprises identifying the subject being at risk for a thrombotic coagulopathy when the VWF activity level is at least about 1.2, 1.4, 1.6, 1.8, 2.0, 2.2, 2.4, 2.6, 2.8, 3.0, 3.2, 3.4, 3.6, 3.8, 4.0, 6.0, 8.0, 9.0, or 10.0 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the VWF activity level is about 1.0 IU/ml to about 9.0 IU/ml, about 1.0 IU/ml to about 4.0 IU/ml, about 1.0 IU/ml to about 3.0 IU/ml, about 1.2 IU/ml to about 9.0 IU/ml, about 1.2 IU/ml to about 4.0 IU/ml, about 1.2 IU/ml to about 3.0 IU/ml, about 1.3 IU/ml to about 9.0 IU/ml, about 1.3 IU/ml to about 4.0 IU/ml, about 1.3 IU/ml to about 3.0 IU/ml, about 1.5 IU/ml to about 9.0 IU/ml, about 1.5 IU/ml to about 4.0 IU/ml, about 1.5 IU/ml to about 3.0 IU/ml, about 1.8 IU/ml to about 9.0 IU/ml, about 1.8 IU/ml to about 4.0 IU/ml, or about 1.8 IU/ml to about 3.0 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the VWF activity level is about 1.0 IU/ml to about 4.4 IU/ml, about 1.0 IU/ml to about 4.0 IU/ml, about 1.0 IU/ml to about 3.3 IU/ml, about 1.0 IU/ml to about 3.0 IU/ml, about 1.2 IU/ml to about 4.4 IU/ml, about 1.2 IU/ml to about 4.0 IU/ml, about 1.2 IU/ml to about 3.3 IU/ml, about 1.2 IU/ml to about 3.0 IU/ml, about 1.3 IU/ml to about 4.4 IU/ml, about 1.3 IU/ml to about 4.0 IU/ml, about 1.3 IU/ml to about 3.3 IU/ml, about 1.3 IU/ml to about 3.0 IU/ml, about 1.5 IU/ml to about 4.4 IU/ml, about 1.5 IU/ml to about 4.0 IU/ml, about 1.5 IU/ml to about 3.3 IU/ml, about 1.5 IU/ml to about 3.0 IU/ml, about 1.8 IU/ml to about 4.4 IU/ml, about 1.8 IU/ml to about 4.0 IU/ml, about 1.8 IU/ml to about 3.3 IU/ml, or about 1.8 IU/ml to about 3.0 IU/ml. In certain embodiments, the method comprises identifying the subject being at risk for a thrombotic coagulopathy when the VWF activity level is about 1.0 IU/ml to about 4.0 IU/ml, 1.0 IU/ml to about 3.0 IU/ml, about 1.2 IU/ml to about 4.0 IU/ml, about 1.2 IU/ml to about 3.0 IU/ml, about 1.3 IU/ml to about 4.0 IU/ml, about 1.3 IU/ml to about 3.0 IU/ml, about 1.8 IU/ml to about 4.0 IU/ml, or about 1.8 IU/ml to about 3.0 IU/ml/ml. In certain embodiments, subjects with this risk are administered lower doses of ADAMTS13 (e.g., about 10-40 IU/kg).

In certain embodiments, the subject is at a high risk for a thrombotic coagulopathy when the VWF activity level is at least about 2.0, 2.5, 3.0, 3.5, 4.0, 4.5, 5.0, 5.5, 6.0, 6.5, 7.0, 7.5, 8.0, 8.5, 9.0, 9.5, or 10.0 IU/ml. In certain embodiments, the subject is at a high risk for a thrombotic coagulopathy when the VWF activity level is at least about 3.0, 3.1, 3.2, 3.3, 3.4, 3.5, 3.6, 3.7, 3.8, 3.9, 4.0, 4.0, 4.2, 4.2, 4.4, 4.5, 4.6, 4.7, 4.8, 4.9, or 5.0 IU/ml. In certain embodiments, the subject is at a high risk for a thrombotic coagulopathy when the VWF activity level is at least about 2.0, 3.0, 3.3, 4.0, 4.4, 4.5, or 5 IU/ml. In certain embodiments, the subject is at a high risk for a thrombotic coagulopathy when the VWF activity level is at least about 3.3 IU/ml or about 4.4 IU/ml. In certain embodiments, subjects with this risk are administered higher doses of ADAMTS13 (e.g., about 40-400 IU/kg).

In certain embodiments, the method comprises identifying the subject being at high risk for a thrombotic coagulopathy when the VWF activity level is at least about 2.5, 2.6, 2.7, 2.8, 2.9, 3.0, 3.1, 3.2, 3.3, 3.4, 3.5, 3.6, 3.7, 3.8, 3.9, 4.0, 5.0, 6.0, 7.0, 8.0, 9.0, or 10.0 IU/ml. In certain embodiments, the method comprises identifying the subject being at high risk for a thrombotic coagulopathy when the VWF activity level is at least about 2.6, 2.8, 3.0, 3.2, 3.4, 3.6, 3.8, 4.0, 6.0, 8.0, 9.0, or 10.0 IU/ml. In certain embodiments, the method comprises identifying the subject being at high risk for a thrombotic coagulopathy when the VWF activity level is at least about 3.0, 3.3, 4.0, 4.4, 9.0, 9.2, 9.4, or 10 IU/ml. In certain embodiments, the method comprises identifying the subject being at high risk for a thrombotic coagulopathy when the VWF activity level is at least about 3.3, 4.4, 9.2 or 9.4 IU/ml. In certain embodiments, subjects with this risk are administered higher doses of ADAMTS13 (e.g., about 40-400 IU/kg).

In certain embodiments, the method comprises identifying the subject being at risk for a thrombotic coagulopathy when plasma UHMW VWF protein multimers are detected. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, subject is at risk for developing a thrombotic coagulopathy when the plasma level of UHMW VWF multimers is about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, or about 129% of the baseline value for said UHMW VWF multimer plasma level. In certain embodiments, subject is at risk for developing a thrombotic coagulopathy when the plasma level of UHMW VWF multimers is at least about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, or about 129% of the baseline value for said UHMW VWF multimer plasma level. In certain embodiments, the subject is at risk for developing a thrombotic coagulopathy when the plasma level of UHMW VWF multimers is about 100-126%, about 100-124%, about 100-122%, about 100-120%, about 100-115%, about 100-114%, about 100-113%, about 100-112%, about 100-111%, about 100-110%, about 100-109%, about 100-108%, about 100-109%, about 100-106%, about 100-105%, about 100-104%, about 100-103%, about 100-102%, or about 100-101% of the baseline value for said UHMW VWF multimer plasma level. In certain embodiments, the subject is at risk for developing a thrombotic coagulopathy when the plasma level of UHMW VWF multimers is about 100-102%, about 100-105%, about 100-110%, about 100-115%, about 100-120%, or about 100-122% of the baseline value for said UHMW VWF multimer plasma level. In certain embodiments, the subject is at risk for developing a thrombotic coagulopathy when the plasma level of UHMW VWF multimers is about 100-110% of the baseline value for said UHMW VWF multimer plasma level. In certain embodiments, the subject is at risk for developing a thrombotic coagulopathy when the plasma level of UHMW VWF multimers is about 100-115% of the baseline value for said UHMW VWF multimer plasma level. In certain embodiments, the subject is at risk for developing a thrombotic coagulopathy when the plasma level of UHMW VWF multimers is about 100-120% of the baseline value for said UHMW VWF multimer plasma level. In certain embodiments, the subject is at risk for developing a thrombotic coagulopathy when the plasma level of UHMW VWF multimers is about 100-122% of the baseline value for said UHMW VWF multimer plasma level. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, subject is at risk for developing a thrombotic coagulopathy when the plasma level of UHMW VWF multimers is about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 130%, about 140%, about 150%, about 160%, about 170%, about 180%, about 190%, about 200%, about 250%, about 300%, about 350%, about 400%, or more of the baseline value for said UHMW VWF multimer plasma level. In certain embodiments, subject is at risk for developing a thrombotic coagulopathy when the plasma level of UHMW VWF multimers is at least about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 130%, about 140%, about 150%, about 160%, about 170%, about 180%, about 190%, about 200%, about 250%, about 300%, about 350%, about 400%, or more of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, subject is at high risk for developing a thrombotic coagulopathy when the plasma level of UHMW VWF multimers is at least about 110%, about 111%, about 112%, about 113%, about 114%, about 115%, about 116%, about 117%, about 118%, about 119%, about 120%, or about 130% of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, subjects with this risk are administered higher doses of ADAMTS13 (e.g., about 40-400 IU/kg).

In certain embodiments, subject is at risk for developing a thrombotic coagulopathy when the plasma level of UHMW VWF multimers is about 100%, about 101%, about 102%, about 103%, about 104%, about 105%, about 106%, about 107%, about 108%, about 109%, about 110%, about 111%, about 112%, about 113%, about 114%, or about 115% of the baseline value for said UHMW VWF multimer plasma level. In certain embodiments, the subject is at risk for developing a thrombotic coagulopathy when the plasma level of UHMW VWF multimers is about 100-102%, about 100-105%, about 100-110%, or about 100-115% of the baseline value for said UHMW VWF multimer plasma level. In certain embodiments, subject is at risk for developing a thrombotic coagulopathy when the plasma level of UHMW VWF multimers is about 110% or more of the baseline value for said UHMW VWF multimer plasma level. In certain embodiments, subjects with this risk are administered lower doses of ADAMTS13 (e.g., about 10-40 IU/kg).

In certain embodiments, subject is at high risk for developing a thrombotic coagulopathy when the plasma level of UHMW VWF multimers is at least about 115%, about 116%, about 117%, about 118%, about 119%, about 120%, about 130%, about 140%, about 150%, about 160%, about 170%, about 180%, about 190%, about 200%, about 250%, about 300%, about 350%, about 400%, or more of the baseline value for said UHMW VWF multimer plasma level. In certain embodiments, the subject is at high risk for developing a thrombotic coagulopathy when the plasma level of UHMW VWF multimers is about 115-120% or about 115-122% of the baseline value for said UHMW VWF multimer plasma level. In certain embodiments, subjects with this risk are administered higher doses of ADAMTS13 (e.g., about 40-400 IU/kg).

In certain embodiments, the method comprises identifying the subject being at risk for a thrombotic coagulopathy when the plasma level of ADAMTS13 protein is no more than about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, or 1.6 IU/ml. In certain embodiments, the method comprises identifying the subject being at risk for a thrombotic coagulopathy when the plasma level of ADAMTS13 protein is less than about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, or 1.6 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the plasma level of ADAMTS13 protein is about 0.06 IU/ml to about 0.8 IU/ml, about 0.06 IU/ml to about 0.7 IU/ml, about 0.06 IU/ml to about 0.6 IU/ml, about 0.06 IU/ml to about 0.5 IU/ml, about 0.06 IU/ml to about 0.4 IU/ml, about 0.06 IU/ml to about 0.3 IU/ml, about 0.07 IU/ml to about 0.8 IU/ml, about 0.07 IU/ml to about 0.7 IU/ml, about 0.07 IU/ml to about 0.6 IU/ml, about 0.07 IU/ml to about 0.5 IU/ml, about 0.07 IU/ml to about 0.4 IU/ml, about 0.07 IU/ml to about 0.3 IU/ml, about 0.08 IU/ml to about 0.8 IU/ml, about 0.08 IU/ml to about 0.7 IU/ml, about 0.08 IU/ml to about 0.6 IU/ml, about 0.08 IU/ml to about 0.5 IU/ml, about 0.08 IU/ml to about 0.4 IU/ml, about 0.08 IU/ml to about 0.3 IU/ml, about 0.1 IU/ml to about 0.8 IU/ml, about 0.1 IU/ml to about 0.7 IU/ml, about 0.1 IU/ml to about 0.6 IU/ml, about 0.1 IU/ml to about 0.5 IU/ml, about 0.1 IU/ml to about 0.4 IU/ml, about 0.40 IU/ml to about 0.8 IU/ml, about 0.40 IU/ml to about 0.7 IU/ml, or about 0.40 IU/ml to about 0.6 IU/ml. In certain embodiments, the method comprises identifying the subject being at risk for a thrombotic coagulopathy when the plasma level of ADAMTS13 protein is about 0.08 IU/ml to about 0.7 IU/ml, about 0.08 IU/ml to about 0.40 IU/ml, or about 0.40 IU/ml to about 0.7 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the plasma level of ADAMTS13 protein is less than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, or 1.6 IU/ml. In certain embodiments, the method comprises identifying the subject being at risk for a thrombotic coagulopathy when the plasma level of ADAMTS13 protein is no more than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, or 1.6 IU/ml. In certain embodiments, the method comprises identifying the subject being at risk for a thrombotic coagulopathy when the plasma level of ADAMTS13 protein is about 0.40 IU/ml to about 0.7 IU/ml, about 0.5 IU/kg to about 0.7 IU/ml, about 0.6 IU/kg to about 0.70 IU/kg. In certain embodiments, subjects with this risk are administered lower doses of ADAMTS13 (e.g., about 10-40 IU/kg).

In certain embodiments, the subject is at a high risk for a thrombotic coagulopathy when the plasma level of ADAMTS13 protein is no more than about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 IU/ml. In certain embodiments, the subject is at a high risk for a thrombotic coagulopathy when the plasma level of ADAMTS13 protein is less than about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 IU/ml. In certain embodiments, the subject is at a high risk for a thrombotic coagulopathy when the plasma level of ADAMTS13 protein is no more than about 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40 IU/ml. In certain embodiments, the subject is at a high risk for a thrombotic coagulopathy when the plasma level of ADAMTS13 protein is nor more than about 0.3, 0.4, or about 0.5 IU/ml. In certain embodiments, the subject is at a high risk for a thrombotic coagulopathy when the plasma level of ADAMTS13 protein is no more than about 0.4 IU/ml. In certain embodiments, subjects with this risk are administered higher doses of ADAMTS13 (e.g., about 40-400 IU/kg).

In certain embodiments, the method comprises identifying the subject being at risk for a thrombotic coagulopathy when the activity level of ADAMTS13 protein is no more than about 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, or 2 IU/ml. In certain embodiments, the method comprises identifying the subject being at risk for a thrombotic coagulopathy when the activity level of ADAMTS13 protein is less than about 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, or 2 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the activity level of ADAMTS13 protein is no more than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 IU/ml. In certain embodiments, the method comprises identifying the subject being at risk for a thrombotic coagulopathy when the activity level of ADAMTS13 protein is less than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 IU/ml. In certain embodiments, the method comprises identifying the subject being at risk for a thrombotic coagulopathy when the activity level of ADAMTS13 protein is no more than about 0.70 IU/ml. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the activity level of ADAMTS13 protein is no more than about 0.2 IU/ml to about 0.9 IU/ml, about 0.2 IU/ml to about 0.8 IU/ml, about 0.2 IU/ml to about 0.5 IU/ml, about 0.2 IU/ml to about 0.4 IU/ml, about 0.4 IU/ml to about 0.9 IU/ml, about 0.4 IU/ml to about 0.8 IU/ml, about 0.4 IU/ml to about 0.5 IU/ml, about 0.5 IU/ml to about 0.9 IU/ml, or about 0.5 IU/ml to about 0.8. In certain embodiments, subjects with this risk are administered lower doses of ADAMTS13 (e.g., about 10-40 IU/kg). In certain embodiments, subjects with this risk are administered higher doses of ADAMTS13 (e.g., about 40-400 IU/kg).

In certain embodiments, the method comprises identifying the subject being at risk for a thrombotic coagulopathy when the activity level of ADAMTS13 protein is no more than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 IU/ml. In certain embodiments, the method comprises identifying the subject being at risk for a thrombotic coagulopathy when the activity level of ADAMTS13 protein is less than about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.68, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 IU/ml. In certain embodiments, the method comprises identifying the subject being at risk for a thrombotic coagulopathy when the activity level of ADAMTS13 protein is no more than about 0.80, 0.81, 0.82, 0.83, 0.84, 0.85, 0.86, 0.87, 0.88, 0.89, or 0.9 IU/ml. In certain embodiments, the method comprises identifying the subject being at risk for a thrombotic coagulopathy when the activity level of ADAMTS13 protein is no more than about 0.80 or 0.9 IU/ml. In certain embodiments, subjects with this risk are administered lower doses of ADAMTS13 (e.g., about 10-40 IU/kg).

In certain embodiments, the subject is at a high risk for a thrombotic coagulopathy when the activity level of ADAMTS13 protein is no more than about 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 IU/ml. In certain embodiments, the subject is at a high risk for a thrombotic coagulopathy when the activity level of ADAMTS13 protein is less than about 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 IU/ml. In certain embodiments, the subject is at a high risk for a thrombotic coagulopathy when the activity level of ADAMTS13 protein is no more than about 0.3 IU/ml, 0.4 IU/ml, about 0.5 IU/ml, or about 0.6 IU/ml. In certain embodiments, the subject is at a high risk for a thrombotic coagulopathy when the activity level of ADAMTS13 protein is less than about 0.3 IU/ml, 0.4 IU/ml, about 0.5 IU/ml, or about 0.6 IU/ml. In certain embodiments, the subject is at a high risk for a thrombotic coagulopathy when the activity level of ADAMTS13 protein is no more than about 0.4 IU/ml or about 0.5 IU/ml. In certain embodiments, the subject is at a high risk for a thrombotic coagulopathy when the activity level of ADAMTS13 protein is less than about 0.4 IU/ml or about 0.5 IU/ml. In certain embodiments, subjects with this risk are administered higher doses of ADAMTS13 (e.g., about 40-400 IU/kg).

In certain embodiments, the predetermined normal baseline is based on a normal control population in the testing laboratory with the validated/chosen methods of measurement. In certain embodiments, when a baseline range is provided, the subject's sample is compared to the upper limit of the range when evaluating an increase compared to normal control. In certain embodiments, when a baseline range is provided, the subject's sample is compared to the lower limit of the range when evaluating an decrease compared to normal control. In certain embodiments, when a baseline range is provided, the subject's sample is compared to the mean, median, or mode of the predetermined baseline range. In certain embodiments, when a baseline range is provided, the subject's sample is compared to the mean of the predetermined baseline range.

In certain embodiments, the subject is diagnosed with COVID by detecting SARS-CoV-2 RNA by PCR from a blood or nasal mucus sample taken from the subject. In certain embodiments, the subject is diagnosed with COVID by SARS-CoV-2 seroconversion. In certain embodiments, the subject is diagnosed with COVID by detection of SARS-CoV-2 antibodies in the subject's plasma.

In certain embodiments, the blood sample is treated with an anticoagulant. In certain embodiments, the anticoagulant is EDTA, sodium citrate, or heparin.

In certain aspects, the invention provides a kit for determining whether a subject diagnosed with COVID-19 is at risk for a thrombotic coagulopathy, said kit comprising (i) one or more reagents for determining one or more of the plasma level of VWF protein, activity level of VWF, plasma level of UHMW VWF multimers, plasma level of ADAMTS13 protein, activity level of ADAMTS13, (ii) optionally packaging and/or instructions for use, and (iii) optionally one or more reagents for detecting SARS-CoV-2 or diagnosing COVID-19.

E. Dosage Amounts

In certain embodiments, the therapeutically effective amount or dose of isolated or recombinant ADAMTS13 is expressed as the number of International Units (IU) of ADAMTS13 activity to be administered, per kg of a subject's body weight (IU/kg). Any suitable method for determining IUs of ADAMTS13 activity is within the scope of the invention, and such methods are known, including FRETS-VWF73 activity, for example as described above.

(1) Dosing ADAMTS13 in IU/kg

In certain embodiments, the therapeutically effective amount or dose of ADAMTS13 is about 10-400 IU/kg. In certain embodiments, the therapeutically effective amount or dose of ADAMTS13 is about 10-320 IU/kg. In certain embodiments, the therapeutically effective amount or dose of ADAMTS13 is about 10-300 IU/kg. In certain embodiments, the therapeutically effective amount or dose of ADAMTS13 is about 10-200 IU/kg. In certain embodiments, the therapeutically effective amount or dose of ADAMTS13 is about 10-160 IU/kg. In certain embodiments, the therapeutically effective amount or dose of ADAMTS13 is about 20-400 IU/kg. In certain embodiments, the therapeutically effective amount or dose of ADAMTS13 is about 20-320 IU/kg. In certain embodiments, the therapeutically effective amount or dose of ADAMTS13 is about 20-300 IU/kg. In certain embodiments, the therapeutically effective amount or dose of ADAMTS13 is about 20-200 IU/kg. In certain embodiments, the therapeutically effective amount or dose of ADAMTS13 is about 20-160 IU/kg. In other embodiments the dose is about 10-180 IU/kg, about 10-160 IU/kg, about 20-400 IU/kg, about 20-320 IU/kg, about 20-300 IU/kg, about 20-200 IU/kg, about 20-180 IU/kg, about 20-160 IU/kg, about 40-400 IU/kg, about 40-320 IU/kg, about 40-300 IU/kg, about 40-200 IU/kg, about 40-180 IU/kg, about 40-160 IU/kg, about 50-400 IU/kg, about 50-320 IU/kg, about 50-300 IU/kg, about 50-200 IU/kg, about 50-180 IU/kg, about 50-160 IU/kg, about 60-400 IU/kg, about 60-320 IU/kg, about 60-300 IU/kg, about 60-200 IU/kg, about 60-180 IU/kg, or about 60-160 IU/kg. In further embodiments, the dose is about 10-100 IU/kg, about 10-80 IU/kg, about 10-60 IU/kg, about 10-40 IU/kg, about 20-100 IU/kg, about 20-80 IU/kg, about 20-60 IU/kg, or about 20-40 IU/kg. In yet other embodiments, the dose is about 30-180 IU/kg, about 30-160 IU/kg, about 30-150 IU/kg, about 40-180 IU/kg, about 40-160 IU/kg, about 40-150 IU/kg, about 50-180 IU/kg, about 50-160 IU/kg, about 50-150 IU/kg, about 60-180 IU/kg, about 60-160 IU/kg, or about 60-150 IU/kg. In certain embodiments, the therapeutically effective amount or dose of ADAMTS13 is about 10 IU/kg, about 20 IU/kg, about 30 IU/kg, about 40 IU/kg, about 50 IU/kg, about 60 IU/kg, about 80 IU/kg, about 100 IU/kg, about 120 IU/kg, about 140 IU/kg, about 160 IU/kg, about 200 IU/kg, about 300 IU/kg, about 320 IU/kg, or about 400 IU/kg. In certain embodiments, the therapeutically effective amount or dose is about 10 IU/kg, about 20 IU/kg, about 40 IU/kg, or about 60 IU/kg. A therapeutically effective amount or dose may be administered as a single dose, as multiple doses, or as divided doses. For example, the therapeutically effective amount or dose may be administered in a singular dose or in multiple doses to maintain a circulating level of total ADAMTS13 effective to treat or prevent the condition. In such aspects, the therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered daily or every other day.

In certain embodiments, therapeutically effective amount or dose is administered immediately upon detection of SARS-CoV-2 infection or diagnosis of COVID-19 or upon determination of risk of, or vulnerability to one or more symptoms, complications, or risk factors associated with COVID-19, e.g., within 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 110 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 or more hours, or any combination thereof.

Certain COVID-19 subjects may be over 65 years of age and/or may present with a history, signs or symptoms of, or a predisposition or susceptibility to, one or more complications or risk factors associated with COVID-19. These include, for example and not for limitation, an elevated levels of VWF and/or its multimers (especially ultralarge multimers (UHMW)), elevated activity levels of VWF, reduced plasma levels of endogenous ADAMTS13, reduced activity of endogenous ADAMTS13, elevated cytokine levels, coagulopathies, blood-clotting disorders, veno-occlusive disorders, prothrombotic conditions, inherited thrombotic thrombocytopenic purpura (TTP), acquired TTP, disseminated intravascular coagulation (DIC), sepsis, sickle cell, renal failure, respiratory failure, acute respiratory distress syndrome (ARDS), chronic obstructive pulmonary disease (COPD), pneumonia, asthma, pregnancy, menopause, pen-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke), or symptoms or complications thereof. These subjects are referred to herein as subjects or patients “at risk.”

Certain “at risk” COVID-19 subjects may present at earlier stages of the disease, with or without COVID-19 symptoms, for example upon testing the subject for a SARS-CoV-2 infection. These subjects may or may not exhibit elevated levels of VWF and/or its multimers (e.g., UHMW); such levels may appear normal or only slightly elevated. According to the invention, such “at risk earlier stage” subjects may be treated by administering a composition comprising a therapeutically effective amount of isolated or recombinant ADAMTS13. In certain embodiments, a therapeutically effective amount would be about 10-100 IU/kg, about 10-80 IU/kg, about 20-100 IU/kg, about 20-80 IU/kg, about 30-100 IU/kg, about 30-80 IU/kg, about 40-100 IU/kg, about 40-80 IU/kg, about 50-100 IU/kg, about 50-80 IU/kg, about 60-100 IU/kg, or about 60-80 IU/kg. In certain embodiments, a therapeutically effective amount would be about 10-60 IU/kg. In certain embodiments, a therapeutically effective amount would be about 10-40 IU/kg. In certain embodiments, a therapeutically effective amount would be about 10-20 IU/kg. In certain embodiments, a therapeutically effective amount would be about 20-60 IU/kg. In certain embodiments, a therapeutically effective amount would be about 20-40 IU/kg. In certain embodiments, a therapeutically effective amount would be about 20-30 IU/kg. In certain embodiments, the therapeutically effective amount or dose is about 10 IU/kg, about 20 IU/kg, about 30 IU/kg, about 40 IU/kg, about 50 IU/kg, or about 60 IU/kg. In certain embodiments, the therapeutically effective amount or dose is about 10 IU/kg, about 20 IU/kg, about 30 IU/kg, or about 40 IU/kg. In certain embodiments, dosing is determined and/or monitored to provide an increase in the subject's circulating ADAMTS13 levels of from about 20-100%, compared to a normal baseline value (e.g., predetermined normal baseline value). In certain embodiments, dosing is determined and/or monitored to provide an increase in the subject's circulating ADAMTS13 levels of from about 100-150%, compared to a predetermined normal baseline value. In certain embodiments, dosing is determined and/or monitored to provide an increase in the subject's circulating ADAMTS13 levels about 100%, about 110%, about 120%, about 125%, about 130%, about 140%, or about 150%, compared to a predetermined normal baseline value. In certain embodiments, dosing is determined and/or monitored to provide an increase in the subject's circulating ADAMTS13 levels about 100%, about 110%, about 120%, about 125%, about 130%, about 140%, or about 150%, compared to a predetermined normal baseline value. In certain embodiments, the composition is administered to “at risk earlier stage” subjects promptly upon a COVID-19 diagnosis or hospitalization, or within 24 or 48 hours of a COVID-19 diagnosis or hospitalization. In certain embodiments, the “at risk” earlier stage subjects their VWF and/or its multimers (e.g., UHMW) levels. VWF and/or its multimers (e.g., UHMW) activity, ADAMTS13 levels and/or ADAMTS13 activity have not been evaluated before administration of ADAMTS13. In such aspects, the therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered daily or every other day. According to the invention, treatment as described herein would treat, inhibit, suppress, prevent, reduce, or alleviate severe progression of one or more COVID-19 complications, particularly the various thrombotic or prothrombotic conditions and complications described herein.

Certain “at risk” COVID-19 patients may present at later stages of the disease, and/or may present with elevated levels of VWF or its multimers (e.g., UHMW). In certain embodiments, elevated VWF or multimer levels indicating treatment would be levels that are at least about 5%, 10%, 20%, 30%, 40%, 50%, 60%, 70%, 80%, 90%, 100%, 200%, 300%, 400%, 500%, 600%, 700%, 800%, 900% above a predetermined normal baseline value. In certain embodiments, treatment is indicated when VWF and/or multimer levels are twice or three times as high, or higher, compared to a predetermined normal baseline value. In certain embodiments, “at risk later stage” COVID-19 patients are those admitted to the Intensive Care Unit (“ICU”). In certain embodiments, “at risk later stage” COVID-19 patients are those that have been intubated. According to the invention, such “at risk later stage” subjects may be treated by administering a composition comprising a therapeutically effective amount of isolated or recombinant ADAMTS13.

In certain embodiments, the predetermined normal baseline is based on a normal control population in the testing laboratory with the validated/chosen methods of measurement.

In certain embodiments, a therapeutically effective amount for a “later stage” COVID-19 subject would be about 20-400 IU/kg, about 320-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, about 30-400 IU/kg, about 30-320 IU/kg, about 30-300 IU/kg, about 30-200 IU/kg, about 30-180 IU/kg, about 30-160 IU/kg, about 30-80 IU/kg, about 30-60 IU/kg, about 30-40 IU/kg, about 40-400 IU/kg, about 40-320 IU/kg, about 40-300 IU/kg, about 40-200 IU/kg, about 40-180 IU/kg, about 40-160 IU/kg, about 40-80 IU/kg, about 40-60 IU/kg, about 50-400 IU/kg, about 50-320 IU/kg, about 50-300 IU/kg, about 50-200 IU/kg, about 50-180 IU/kg, about 50-160 IU/kg, about 50-80 IU/kg, about 50-60 IU/kg, about 60-400 IU/kg, about 60-320 IU/kg, about 60-300 IU/kg, about 60-200 IU/kg, about 60-180 IU/kg, about 60-160 IU/kg, or about 60-80 IU/kg. In certain embodiments, a therapeutically effective amount would be about 30-320 IU/kg. In certain embodiments, a therapeutically effective amount would be about 30-160 IU/kg. In certain embodiments, a therapeutically effective amount would be about 30-80 IU/kg. In certain embodiments, a therapeutically effective amount would be about 30-60 IU/kg. In certain embodiments, a therapeutically effective amount would be about 40-320 IU/kg. In certain embodiments, a therapeutically effective amount would be about 40-160 IU/kg. In certain embodiments, a therapeutically effective amount would be about 40-80 IU/kg. In certain embodiments, a therapeutically effective amount would be about 40-60 IU/kg. In certain embodiments, the therapeutically effective amount or dose is about 20 IU/kg, about 30 IU/kg, about 40 IU/kg, about 60 IU/kg, about 80 IU/kg, or about 160 IU/kg. In certain embodiments, the therapeutically effective amount or dose is about 40 IU/kg, about 80 IU/kg, or about 160 IU/kg. In certain embodiments, dosing is determined and/or monitored to provide a reduction in levels of VWF and/or its multimers (e.g., UHMW) that are within or approximate a predetermined normal range or predetermined baseline value. In certain embodiments, ultralarge VWF multimers will no longer be observed. In certain embodiments, the ADAMTS13 composition is administered to “at risk later stage” subjects promptly upon a COVID-19 diagnosis or hospitalization, or within 24 or 48 hours of a COVID-19 diagnosis or hospitalization. In certain embodiments, the “at risk” earlier stage subjects their VWF and/or its multimers (e.g., UHMW) levels. VWF and/or its multimers (e.g., UHMW) activity, ADAMTS13 levels and/or ADAMTS13 activity have not been evaluated before administration of ADAMTS13. In such aspects, the therapeutically effective amount or dose is administered monthly, every two weeks, weekly, twice a week, three times a week, every other day, daily, every 12 hours, every 8 hours, every six hours, every four hours, every two hours, or every hour. In certain embodiments, a therapeutically effective amount or dose is administered daily or every other day According to the invention, treatment as described herein would treat, inhibit, suppress, prevent, reduce, or alleviate severe progression of one or more COVID-19 complications, particularly the various thrombotic or prothrombotic conditions and complications described herein.

Without wishing to be bound by any theory, it is believed that “at risk later stage” COVID-19 subjects would tend to benefit from administration of higher doses of ADAMTS13, administered more frequently, than doses administered to “at risk earlier stage” C OVID-19 subjects.

In certain embodiments, the dose is about 10-400 IU/kg. In certain embodiments, the dose is about 10-320 IU/kg. In certain embodiments, the dose is about 10-300 IU/kg. In certain embodiments the dose is about 10-200 IU/kg. In certain embodiments, the dose is about 10-300 IU/kg. In certain embodiments the dose is about 10-160 IU/kg. In certain embodiments the dose is about 10-180 IU/kg, about 10-160 IU/kg, about 20-400 IU/kg, about 20-320 IU/kg, about 20-300 IU/kg, about 20-200 IU/kg, about 20-180 IU/kg, about 20-160 IU/kg, about 40-400 IU/kg, about 40-320 IU/kg, about 40-300 IU/kg, about 40-200 IU/kg, about 40-180 IU/kg, about 40-160 IU/kg, about 50-400 IU/kg, about 50-320 IU/kg, about 50-300 IU/kg, about 50-200 IU/kg, about 50-180 IU/kg, about 50-160 IU/kg, about 60-400 IU/kg, about 60-320 IU/kg, about 60-300 IU/kg, about 60-200 IU/kg, about 60-180 IU/kg, or about 60-160 IU/kg. In certain embodiments, the dose is about 10-100 IU/kg, about 10-80 IU/kg, about 10-60 IU/kg, 10-60 IU/kg, about 20-100 IU/kg, about 20-80 IU/kg, about 20-60 IU/kg, or about 20-40 IU/kg. In certain embodiments, the dose is about 30-180 IU/kg, about 30-160 IU/kg, about 30-150 IU/kg, about 40-180 IU/kg, about 40-160 IU/kg, about 40-150 IU/kg, about 50-180 IU/kg, about 50-160 IU/kg, about 50-150 IU/kg, about 60-180 IU/kg, about 60-160 IU/kg, or about 60-150 IU/kg. In certain embodiments, the dose is about 10 IU/kg, about 20 IU/kg, about 30 IU/kg, about 40 IU/kg, about 50 IU/kg, about 60 IU/kg, about 80 IU/kg, about 100 IU/kg, about 120 IU/kg, about 140 IU/kg, about 160 IU/kg, about 200 IU/kg, about 300 IU/kg, about 320 IU/kg, or about 400 IU/kg. In certain embodiments, the therapeutically effective amount or dose is about 10 IU/kg, about 20 IU/kg, about 40 IU/kg, about 60 IU/kg, about 80 IU/kg, or about 160 IU/kg.

In certain embodiments the therapeutically effective amount or dose is at least about 10 IU/kg, at least about 20 IU/kg, at least about 30 IU/kg, at least about 40 IU/kg, at least about 50 IU/kg, at least about 60 IU/kg, at least about 70 IU/kg, at least about 80 IU/kg, at least about 90 IU/kg, at least about 100 IU/kg, at least about 110 IU/kg, at least about 120 IU/kg, at least about 130 IU/kg, at least about 140 IU/kg, at least about 150 IU/kg, or at least about 160 IU/kg. In certain embodiments the therapeutically effective amount or dose is at least about 10 IU/kg, at least about 20 IU/kg, at least about 40 IU/kg, or at least about 60 IU/kg. In certain embodiments the dose is about 10-200 IU/kg. In certain embodiments the dose is about 10-180 IU/kg, about 10-160 IU/kg, about 20-200 IU/kg, about 20-180 IU/kg, or about 20-160 IU/kg. In certain embodiments, the dose is about 10-100 IU/kg, about 10-80 IU/kg, about 10-60 IU/kg, about 10-40 IU/kg, about 20-100 IU/kg, about 20-80 IU/kg, about 20-60 IU/kg, or about 20-40 IU/kg. In certain embodiments, the dose is about 30-180 IU/kg, about 30-160 IU/kg, about 30-150 IU/kg, about 40-180 IU/kg, about 40-160 IU/kg, or about 40-150 IU/kg.

In certain embodiments the therapeutically effective amount or dose is about 10-400 IU/kg, about 10-320 IU/kg, about 10-300 IU/kg, about 10-200 IU/kg, about 10-190 IU/kg, about 10-180 IU/kg, about 10-170 IU/kg, about 10-160 IU/kg, about 10-150 IU/kg, about 10-140 IU/kg, about 10-130 IU/kg, about 10-120 IU/kg, about 10-110 IU/kg, about 10-100 IU/kg, about 10-90 IU/kg, about 10-80 IU/kg, about 10-70 IU/kg, about 10-60 IU/kg, about 10-50 IU/kg, about 10-40 IU/kg, about 10-30 IU/kg, or about 10-20 IU/kg. In certain embodiments the therapeutically effective amount or dose is about 10-320 IU/kg, about 10-160 IU/kg, 10-80 IU/kg, about 10-60 IU/kg, about 10-40 IU/kg, or about 10-20 IU/kg.

In certain embodiments the therapeutically effective amount or dose is about 20-400 IU/kg, about 20-320 IU/kg, about 20-300 IU/kg, about 20-200 IU/kg, about 20-190 IU/kg, about 20-180 IU/kg, about 20-170 IU/kg, about 20-160 IU/kg, about 20-150 IU/kg, about 20-140 IU/kg, about 20-130 IU/kg, about 20-120 IU/kg, about 20-110 IU/kg, about 20-100 IU/kg, about 20-90 IU/kg, about 20-80 IU/kg, about 20-70 IU/kg, about 20-60 IU/kg, about 20-50 IU/kg, about 20-40 IU/kg, or about 20-30 IU/kg. In certain embodiments the therapeutically effective amount or dose is about 20-320 IU/kg, about 20-160 IU/kg, about 20-80 IU/kg, about 20-60 IU/kg, or about 20-40 IU/kg.

In certain embodiments the therapeutically effective amount or dose is about 30-400 IU/kg, about 30-320 IU/kg, about 30-300 IU/kg, about 30-200 IU/kg, about 30-190 IU/kg, about 30-180 IU/kg, about 30-170 IU/kg, about 30-160 IU/kg, about 30-150 IU/kg, about 30-140 IU/kg, about 30-130 IU/kg, about 30-120 IU/kg, about 30-110 IU/kg, about 30-100 IU/kg, about 30-90 IU/kg, about 30-80 IU/kg, about 30-70 IU/kg, about 30-60 IU/kg, about 30-50 IU/kg, or about 30-40 IU/kg. In certain embodiments the therapeutically effective amount or dose is about 30-320 IU/kg, about 30-160 IU/kg, about 30-80 IU/kg, about 30-60 IU/kg, or about 30-40 IU/kg.

In certain embodiments the therapeutically effective amount or dose is about 40-400 IU/kg, about 40-320 IU/kg, about 40-300 IU/kg, about 40-200 IU/kg, about 40-190 IU/kg, about 40-180 IU/kg, about 40-170 IU/kg, about 40-160 IU/kg, about 40-150 IU/kg, about 40-140 IU/kg, about 40-130 IU/kg, about 40-120 IU/kg, about 40-110 IU/kg, about 40-100 IU/kg, about 40-90 IU/kg, about 40-80 IU/kg, about 40-70 IU/kg, about 40-60 IU/kg, or about 40-50 IU/kg. In certain embodiments the therapeutically effective amount or dose is about 40-320 IU/kg, about 40-160 IU/kg, about 40-80 IU/kg, or about 40-60 IU/kg.

In certain embodiments the therapeutically effective amount or dose is about 50-400 IU/kg, about 50-320 IU/kg, about 50-300 IU/kg, about 50-200 IU/kg, about 50-190 IU/kg, about 50-180 IU/kg, about 50-170 IU/kg, about 50-160 IU/kg, about 50-150 IU/kg, about 50-140 IU/kg, about 50-130 IU/kg, about 50-120 IU/kg, about 50-110 IU/kg, about 50-100 IU/kg, about 50-90 IU/kg, about 50-80 IU/kg, about 50-70 IU/kg, or about 50-60 IU/kg. In certain embodiments the therapeutically effective amount or dose is about 50-320 IU/kg, about 50-160 IU/kg, about 50-80 IU/kg, or about 50-60 IU/kg.

In certain embodiments the therapeutically effective amount or dose is about 60-400 IU/kg, about 60-320 IU/kg, about 60-300 IU/kg, about 60-200 IU/kg, about 60-190 IU/kg, about 60-180 IU/kg, about 60-170 IU/kg, about 60-160 IU/kg, about 60-150 IU/kg, about 60-140 IU/kg, about 60-130 IU/kg, about 60-120 IU/kg, about 60-110 IU/kg, about 60-100 IU/kg, about 60-90 IU/kg, about 60-80 IU/kg, or about 60-70 IU/kg. In certain embodiments the therapeutically effective amount or dose is about 60-400 IU/kg, about 60-320 IU/kg, about 60-160 IU/kg, or about 60-80 IU/kg.

In certain embodiments the therapeutically effective amount or dose is about 70-400 IU/kg, about 70-320 IU/kg, about 70-300 IU/kg, about 70-200 IU/kg, about 70-190 IU/kg, about 70-180 IU/kg, about 70-170 IU/kg, about 70-160 IU/kg, about 70-150 IU/kg, about 70-140 IU/kg, about 70-130 IU/kg, about 70-120 IU/kg, about 70-110 IU/kg, about 70-100 IU/kg, about 70-90 IU/kg, or about 70-80 IU/kg. In certain embodiments the therapeutically effective amount or dose is about 70-320 IU/kg, about 70-160 IU/kg, or about 70-80 IU/kg.

In certain embodiments the therapeutically effective amount or dose is about 80-400 IU/kg, about 80-320 IU/kg, about 80-300 IU/kg, about 80-200 IU/kg, about 80-190 IU/kg, about 80-180 IU/kg, about 80-170 IU/kg, about 80-160 IU/kg, about 80-150 IU/kg, about 80-140 IU/kg, about 80-130 IU/kg, about 80-120 IU/kg, about 80-110 IU/kg, about 80-100 IU/kg, or about 80-90 IU/kg. In certain embodiments the therapeutically effective amount or dose is about 80-320 IU/kg or about 80-160 IU/kg.

In certain embodiments the therapeutically effective amount or dose is about 90-400 IU/kg, about 90-320 IU/kg, about 90-300 IU/kg, about 90-200 IU/kg, about 90-190 IU/kg, about 90-180 IU/kg, about 90-170 IU/kg, about 90-160 IU/kg, about 90-150 IU/kg, about 90-140 IU/kg, about 90-130 IU/kg, about 90-120 IU/kg, about 90-110 IU/kg, or about 90-100 IU/kg. In certain embodiments the therapeutically effective amount or dose is about 90-320 IU/kg or about 90-160 IU/kg.

In certain embodiments the therapeutically effective amount or dose is about 100-400 IU/kg, about 100-320 IU/kg, about 100-300 IU/kg, about 100-200 IU/kg, about 100-190 IU/kg, about 100-180 IU/kg, about 100-170 IU/kg, about 100-160 IU/kg, about 100-150 IU/kg, about 100-140 IU/kg, about 100-130 IU/kg, about 100-120 IU/kg, or about 100-110 IU/kg. In certain embodiments the therapeutically effective amount or dose is about 100-400 IU/kg, about 100-320 IU/kg or about 100-160 IU/kg.

In certain embodiments the therapeutically effective amount or dose is about 110-400 IU/kg, about 110-320 IU/kg, about 110-300 IU/kg, about 110-200 IU/kg, about 110-190 IU/kg, about 110-180 IU/kg, about 110-170 IU/kg, about 110-160 IU/kg, about 110-150 IU/kg, about 110-140 IU/kg, about 110-130 IU/kg, or about 110-120 IU/kg. Further embodiments provide doses of about 120-400 IU/kg, about 120-320 IU/kg, about 120-300 IU/kg, about 120-200 IU/kg, about 120-190 IU/kg, about 120-180 IU/kg, about 120-170 IU/kg, about 120-160 IU/kg, about 120-150 IU/kg, about 120-140 IU/kg, or about 120-130 IU/kg.

In certain embodiments the therapeutically effective amount or dose is about 130-400 IU/kg, about 130-320 IU/kg, about 130-300 IU/kg, about 130-200 IU/kg, about 130-190 IU/kg, about 130-180 IU/kg, about 130-170 IU/kg, about 130-160 IU/kg, about 130-150 IU/kg, or about 130-140 IU/kg. Further embodiments provide doses of about 140-400 IU/kg, about 140-320 IU/kg, about 140-300 IU/kg, about 140-200 IU/kg, about 140-190 IU/kg, about 140-180 IU/kg, about 140-170 IU/kg, about 140-160 IU/kg, or about 140-150 IU/kg.

In certain embodiments the therapeutically effective amount or dose is about 150-400 IU/kg, about 150-320 IU/kg, about 150-300 IU/kg, about 150-200 IU/kg, about 150-190 IU/kg, about 150-180 IU/kg, about 150-170 IU/kg, about or 150-160 IU/kg. Further embodiments provide doses of about 160-400 IU/kg, about 160-320 IU/kg, about 160-300 IU/kg, about 160-200 IU/kg, about 160-190 IU/kg, about 160-180 IU/kg, or about 160-170 IU/kg. Yet further embodiments provide doses of about 170-400 IU/kg, about 170-320 IU/kg, about 170-300 IU/kg, about 170-200 IU/kg, about 170-190 IU/kg, or about 170-180 IU/kg. In other embodiments the dose is about 180-200 IU/kg or about 180-190 IU/kg. Another embodiment provides a dose of about 190-200 IU/kg.

Upon improvement of a subject's condition, a maintenance dose may be administered, if necessary. Accordantly, the dosage or frequency of administration, or both, may be reduced, as a function of the symptoms, to a level at which the improved condition is retained. Subjects may, however, require intermittent treatment on a long-term basis upon any recurrence of disease symptoms. In certain embodiments, a maintenance dose would be about 10-60 IU/kg, about 10-40 IU/kg, about 10-30 IU/kg, or about 10-20 IU/kg. In certain embodiments, a maintenance dose would be about 10-20 IU/kg. In certain embodiments, a maintenance dose is at least about 10 IU/kg, at least about 20 IU/kg, at least about 30 IU/kg, at least about 40 IU/kg, at least about 50 IU/kg, or at least about 60 IU/kg. In certain embodiments, a maintenance dose is about 10 IU/kg, about 20 IU/kg, about 30 IU/kg, about 40 IU/kg, about 50 IU/kg, or about 60 IU/kg. In certain embodiments, a maintenance dose is about 10 IU/kg or about 20 IU/kg. In certain embodiments, the maintenance dose is administered monthly, every two weeks, weekly, twice a week, three times a week, every other day, daily, every 12 hours, every 8 hours, every six hours, every four hours, every two hours, or every hour. In certain embodiments, the maintenance dose is administered daily or every other day.

(2) Dosing ADAMTS13 in mg/mL

In certain embodiments, ADAMTS13 is provided in a therapeutically effective dose between about 0.05 mg/mL and about 10 mg/mL. In certain embodiments, ADAMTS13 is provided in a therapeutically effective dose between about 0.05 mg/mL and about 10 mg/mL. In other embodiments, ADAMTS13 is present at a concentration of between about 0.1 mg/mL and about 10 mg/mL. In yet other embodiments, ADAMTS13 is present at a concentration of between about 0.1 mg/mL and about 5 mg/mL. In another embodiment, ADAMTS13 is present at a concentration of between about 0.1 mg/mL and about 2 mg/mL. In yet other embodiments, ADAMTS13 may be present at about 0.01 mg/mL, or at about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, and ranges thereof. In other embodiments the concentration is higher than 10 mg/mL.

(3) Dosing ADAMTS13 in IU/mL

Similarly, in certain embodiments, the concentration of ADAMTS13 may be expressed as an enzymatic activity per unit volume, for example, A13 enzymatic units per mL (IU/mL). For example, in one embodiment a formulation may contain between about 0.01 IU/mL and about 10,000 IU/mL. In another embodiment a formulation may contain between about 0.1 IU/mL and about 10,000 IU/mL. In another embodiment a formulation may contain between about 1 IU/mL and about 10,000 IU/mL. In another embodiment, a formulation may contain between about 10 IU/mL and about 10,000 IU/mL. In other embodiments, the formulation may contain between about 20 IU/mL and about 8,000 IU/mL, or between about 30 IU/mL and about 6,000 IU/mL, or between about 40 IU/mL and about 4,000 IU/mL, or between about 50 IU/mL and about 3,000 IU/mL, or between about 75 IU/mL and about 2,500 IU/mL, or between about 100 IU/mL and about 2,000 IU/mL, or between about 200 IU/mL and about 1,500 IU/mL, or between about other ranges therein. In a preferred embodiment, an ADAMTS13 formulation provided herein contains between about 150 IU/mL and about 600 IU/mL. In another preferred embodiment, an ADAMTS13 formulation provided herein contains between about 100 IU/mL and about 1,000 IU/mL. In certain embodiments, a formulation contains about 0.01 IU/mL, or about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 IU/mL. In certain embodiments, a formulation contains about 10 IU/mL, or about 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more IU/mL.

(4) Dosing ADAMTS13 in Units of FRETS-VWF73 Activity

In yet other embodiments, the concentration of ADAMTS13 in a formulation provided by the present invention may be expressed as a level of enzymatic activity. For example, in one embodiment an ADAMTS13 formulation may contain between about 0.01 units of FRETS-VWF73 activity and about 10,000 units of FRETS-VWF73 activity or other suitable ADAMTS enzymatic unit (IU). In certain embodiments, an ADAMTS13 formulation may contain between about 10 units of FRETS-VWF73 activity and about 10,000 units of FRETS-VWF73 activity or other suitable ADAMTS enzymatic unit (IU). In other embodiments, the formulation may contain between about 20 units of FRETS-VWF73 (U_{FV73}) activity and about 8,000 units of FRETS-VWF73 activity, or between about 30 U_{FV73} and about 6,000 U_{FV73}, or between about 40 U_{FV73} and about 4,000 U_{FV73}, or between about 50 U_{FV73} and about 3,000 U_{FV73}, or between about 75 U_{FV73} and about 2,500 U_{FV73}, or between about 100 U_{FV73} and about 2,000 U_{FV73}, or between about 200 U_{FV73} and about 1,500 U_{FV73}, or between about other ranges therein. In a preferred embodiment, an ADAMTS13 formulation provided herein contains between about 150 and about 600 U_{FV73}. In certain embodiments, a formulation contains about 0.01 units of FRETS VWF73 activity, or about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 units of FRETS VWF73 activity. In certain embodiments, a formulation contains about 10 units of FRETS VWF73 activity, or about 20, 30, 40, 50, 60, 70, 80, 90, 100, 150, 200, 250, 300, 350, 400, 450, 500, 600, 700, 800,900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more units of FRETS-VWF73 activity.

(5) Other Dosing Considerations

In other aspects and embodiments, a therapeutically effective amount of isolated or recombinant ADAMTS13 is administered to a COVID-19 subject upon an assessment that the subject exhibits abnormally high or supernormal level of VWF or ultralarge VWF multimers. In other aspects and embodiments, a therapeutically effective amount of isolated or recombinant ADAMTS13 is administered to a COVID-19 subject upon an assessment that the subject exhibits an abnormally high or supernormal level of VWF or ultralarge VWF multimers. Further aspects and embodiments provide a therapeutically effective amount of isolated or recombinant ADAMTS13 to a COVID-19 subject upon an assessment that the subject exhibits an abnormally low or ultra-low level of ADAMTS13.

In certain embodiments, the amount of ADAMTS13 that is administered to the subject is measured as an increase in the amount of ADAMTS13 in the subject as compared to a control (e.g., the amount of ADAMTS13 in the subject prior to administration). In some embodiments, the ADAMTS13 is administered to the subject at an amount that increases the level of the ADAMTS13 in the subject 1.1, 1.2, 1.3, 1.4, 1.5, 1.6, 1.7, 1.8, 1.9, 2, 3, 4, 5, 6, 7, 8, 9, 10, 15, or 20-fold greater than the level of ADAMTS13 protein in the subject prior to the administering. In some embodiments, the ADAMTS13 protein is administered to the subject at an amount that increases the level of the ADAMTS13 protein at least 5%, 10%, 15%, 20%, 25%, 30%, 35%, 40%, 45%, 50%, 55%, 60%, 65%, 70%, 75%, 80%, 85%, 90%, 95% or 99% greater than the level of ADAMTS13 protein in the subject prior to the administering.

In certain embodiments, the ADAMTS13 or a composition comprising ADAMTS13, is administered in a single bolus injection, every other day, daily, every 12 hours, every 8 hours, every six hours, every four hours, every two hours, or every hour.

In certain embodiments, the ADAMTS13 or composition comprising ADAMTS13, is administered intravenously or subcutaneously.

In certain embodiments, the bioavailability of the ADAMTS13 after subcutaneous administration is at least about 40%, or at least about 45%, or at least about 50%, or at least about 51%, or at least about 52%, or at least about 53%, or at least about 54%, or at least about 55%, or at least about 56%, or at least about 57%, or at least about 58%, or at least about 59%, or at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85% as compared to intravenous administration normalized for the same dose.

In certain embodiments, the bioavailability of the ADAMTS13 after subcutaneous administration is between about 30% to about 90% or about 80% or about 50% as compared to intravenous administration normalized for the same dose. In certain embodiments, the bioavailability of the ADAMTS13 after subcutaneous administration is between about 60% and about 80% as compared to intravenous administration normalized for the same dose. In certain embodiments, the bioavailability of the ADAMTS13 after subcutaneous administration is between about 50% and about 70% as compared to intravenous administration normalized for the same dose. In certain embodiments, the bioavailability of the ADAMTS13 after subcutaneous administration is between about 55% and about 70% as compared to intravenous administration normalized for the same dose. In certain embodiments, the bioavailability of the ADAMTS13 after subcutaneous administration is between about 55% and about 65% as compared to intravenous administration normalized for the same dose. In certain embodiments, the bioavailability of the ADAMTS13 after subcutaneous administration is about 65% as compared to intravenous administration normalized for the same dose.

In certain embodiments, the bioavailability of the ADAMTS13 after subcutaneous administration is about 65% as compared to intravenous administration normalized for the same dose. Thus, in certain embodiments, if the therapeutically effective amount of total ADAMTS13 comprises at least 20-160 international units per kilogram (IU/kg) body weight via intravenous administration, and the bioavailability is 65%, if ±15% variation is applied, there would be 40-80% bioavailability resulting in a 25-400 international units range when administered subcutaneously.

Doses can also be determined based on whether the ADAMTS13 is administered prophylactically (e.g., in repeated doses) or in response to a medical emergency, to immediately reduce harmful effects of an infarction.

It must be kept in mind that the compositions and methods of the present invention can be employed in serious disease states, that is, life-threatening or potentially life-threatening situations. In such cases, in view of the lack of side effects (e.g., hemorrhage, immune system effects), it is possible and may be felt desirable by the treating physician to administer substantial excesses of the pharmaceutical compositions of the invention.

F. Liquid and Lyophilized Formulations and Dosage Forms

The present invention provides stabilized formulations of ADAMTS13. In certain embodiments, the formulations of the invention are stable for at least about 6, months when stored at temperatures up to at least about 40° C. In certain embodiments the formulations are stable for up to 6, 12, or 24 months when stored at temperatures up to at least about 4° C. In other embodiments, the formulations provided herein retain significant ADAMTS13 activity when stored for extended periods of time. In yet other embodiments, the formulations of the invention reduce or retard dimerization, oligomerization, and/or aggregation of an ADAMTS13 protein.

In one embodiment, the present invention provides formulations of ADAMTS13 comprising a therapeutically effective amount or dose of an ADAMTS13, a sub-physiological to physiological concentration of a pharmaceutically acceptable salt, a stabilizing concentration of one or more sugars and/or sugar alcohols, a non-ionic surfactant, a buffering agent providing a neutral pH to the formulation, and optionally a calcium and/or zinc salt. Generally, the stabilized ADAMTS13 formulations provided herein are suitable for pharmaceutical administration. In a preferred embodiment, the ADAMTS13 is human ADAMTS13 or a biologically active derivative or fragment thereof. For example, stabilized ADAMTS13 formulations provided by the present invention will contain a sub-physiological to physiological salt concentration, for example, between and 0 mM and about 200 mM of a pharmaceutically acceptable salt. In one embodiment, an ADAMTS13 formulation will contain a physiological concentration of salt, for example, between about 100 mM and about 200 mM of a pharmaceutically acceptable salt. In other embodiments, an ADAMTS13 formulation will contain about 0 mM, or about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, or more of a pharmaceutically acceptable salt. In a preferred embodiment, the salt is sodium or potassium chloride.

In certain embodiments, the ADAMTS13 formulations are liquid formulations. In other embodiments, the ADAMTS13 formulations are lyophilized from a liquid formulation, according to methods known in the art.

ADAMTS13 formulations containing a sub-physiological concentration of a pharmaceutically acceptable salt form compact lyocakes with smooth surfaces. Furthermore, it has been found that low salt lyophilized formulations of ADAMTS13 proteins reduce protein aggregation as compared to formulations prepared with physiological concentrations of salt. Accordingly, in a preferred embodiment, particularly for lyophilization, the invention provides low salt formulations of ADAMTS13 containing a sub-physiological concentration of a pharmaceutically acceptable salt, for example, less than about 100 mM of a pharmaceutically acceptable salt. In other low salt embodiments, particularly for lyophilization, the formulation contains less than about 80 mM, less than about 60 mM, and about 30 to 60 mM of a pharmaceutically acceptable salt.

In certain embodiments the formulation includes moderate levels (i.e., between about 2% and about 6%) of one or more sugars and/or sugar alcohols to assists in the preparation of compact lyocakes with smooth surfaces and to help stabilize ADAMTS13 upon lyophilization. Accordingly, in one embodiment, the invention provides ADAMTS13 formulations containing between about 2% and about 6% of one or more sugars and/or sugar alcohols. Any sugar such as mono-, di-, or polysaccharides, or water-soluble glucans, including for example fructose, glucose, mannose, sorbose, xylose, maltose, lactose, sucrose, dextran, trehalose, pullulan, dextrin, cyclodextrin, soluble starch, hydroxyethyl starch, and carboxymethylcellulose may be used. In a particular embodiment, sucrose or trehalose is used as a sugar additive. Sugar alcohols are defined as a hydrocarbon having between about 4 and about 8 carbon atoms and a hydroxyl group. Non-limiting examples of sugar alcohols that may be used in the ADAMTS13 formulations provided herein include, mannitol, sorbitol, inositol, galactitol, dulcitol, xylitol, and arabitol. In one embodiment, mannitol is used as a sugar alcohol additive. In a preferred embodiment, an ADAMTS13 formulation contains both a sugar and a sugar alcohol additive.

The sugars and sugar alcohols may be used individually or in combination. In some embodiments, the sugar, sugar alcohol, or combination thereof will be present in the formulation at a concentration of between about 0.5% and about 7%. In one embodiment, the sugar and/or sugar alcohol content of the formulation will be between about 0.5% and about 5%. In certain embodiments, the sugar, sugar alcohol, or combination thereof will be present at a concentration of between about 1% and about 5%. In a preferred embodiment, the sugar, sugar alcohol, or combination thereof will be present at a concentration of between about 2% and about 6%. In another preferred embodiment, the sugar, sugar alcohol, or combination thereof will be present at a concentration of between about 3% and about 5%. In certain embodiments, the final concentration may be about 0.5%, 1%, 1.5%, 2%, 2.5%, 3%, 3.5%, 4%, 4.5%, 5%, 5.5%, 6.0%, 6.5%, or 7.0% sugar, sugar alcohol, or combination thereof. In particular embodiments, a formulation provided herein may comprise a sugar at a concentration from about 0.5% to about 5.0% and a sugar alcohol at a concentration from about 0.5% to about 5.0%. Any combination of sugar and sugar alcohol concentrations may be used, e.g. a sugar present at a concentration of about 0.5%, 1%, 1.5%, 2%, 2.5%, 3%, 3.5%, 4%, 4.5%, 5%, 5.5%, 6.0%, 6.5%, or 7.0% and a sugar alcohol present at a concentration of about 0.5%, 1%, 1.5%, 2%, 2.5%, 3%, 3.5%, 4%, 4.5%, 5%, 5.5%, 6.0%, 6.5%, or 7.0%.

In one embodiment, ADAMTS13 formulations containing a stabilizing concentration of a non-ionic detergent are provided. Pharmaceutically acceptable nonionic surfactants that may be used in the formulations of the present invention are known in the art of pharmaceutical science, and include, without limitation, Polysorbate 80 (Tween 80), Polysorbate 20 (Tween 20), and various poloxamers or pluronics, including Pluronic F-68, and BRIJ 35, or mixtures thereof. In a preferred embodiment, the nonionic surfactant used in the present pharmaceutical formulations is Polysorbate 80. In certain embodiments, a surfactant may be used in a formulation provided herein at a concentration between about 0.001% and about 0.2%. In a preferred embodiment, the surfactant is used at a concentration of between about 0.01% and about 0.1%. In another preferred embodiment, the surfactant is used at a concentration of about 0.05%. For example, in certain embodiments, the formulation may include a nonionic surfactant at a concentration of about 0.001%, 0.005%, 0.01%, 0.02%, 0.03%, 0.04%, 0.05%, 0.06%, 0.07%, 0.08%, 0.09%, 0.1%, 0.125%, 0.15%, 0.175%, 0.2%, and the like.

Furthermore, it was found that ADAMTS13 formulations were stabilized when formulated at a neutral pH between about 6.5 and about 7.5. Accordingly, in certain embodiments, ADAMTS13 formulations are provided that contain a buffering agent suitable to maintain the formulation at a neutral pH. Pharmaceutically acceptable buffering agents are well known in the art, and include without limitation, phosphate buffers, histidine, sodium citrate, HEPES, Tris, Bicine, glycine, N-glycylglycine, sodium acetate, sodium carbonate, glycylglycine, lysine, arginine, sodium phosphate, and mixtures thereof. In preferred embodiments, the buffer is selected from histidine, phosphate buffer, HEPES, and sodium citrate. In one preferred embodiment, the buffer is histidine or HEPES. In a specific embodiment, the buffer is histidine. In another specific embodiment, the buffer is HEPES. In one embodiment, the pH of the formulations provided herein is between about 6.5 and about 9.0. In certain embodiments, the pH of the formulation is about 6.5 or about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In a preferred embodiment, the pH of the A13 formulation is between about 6.0 and about 8.0. In a more preferred embodiment, the pH of the A13 formulation is between about 6.5 and about 7.5. In another embodiment, the pH is about 7.5. In a particular embodiment, the pH of the A13 formulation is about 7.0. In another particular embodiment, the pH of the A13 formulation is 7.0±0.2.

The inclusion of calcium, in any suitable form, may also stabilize the formulation, for example at a concentration from about 0.5 mM to about 10 mM. In another embodiment, calcium is present in an ADAMTS13 formulation at a concentration between about 2 mM and about 5 mM. In a preferred embodiment, calcium is present in an ADAMTS13 formulation at a concentration from about 2 mM to about 4 mM. In certain embodiments, the concentration of calcium is about 0.5 mM, or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In a particular embodiment, the concentration of calcium is about 2 mM. In another preferred embodiment, the concentration of calcium is about 3 mM. In yet another preferred embodiment, the concentration of calcium is about 4 mM.

The inclusion of zinc, in any suitable form, may also stabilize the formulation, for example at a concentration from about 2 μM and about 10 μM. In some embodiments, zinc is present in an ADAMTS13 formulation of the invention at a concentration from about 0.5 μM to about 20.0 μM. In a preferred embodiment, zinc is included in an ADAMTS13 formulation at a concentration of between about 0.5 μM to about 10.0 μM. In certain embodiments, the concentration of zinc is about 0.5 μM, or about 1 μM, 2 μM, 3 μM, 4 μM, 5 μM, 6 μM, 7 μM, 8 μM, 9 μM, or 10 μM.

In certain embodiments, ADAMTS13 formulations provided herein will have a tonicity in a range between about 200 mOsmol/L and about 400 mOsmol/L, or in a range between about 250 and about 350 mOsmol/L. In certain embodiments, an ADAMTS13 formulation provided herein will have a tonicity, for example, of about 200 mOsmol/L, or of about 210 mOsmol/L, 220 mOsmol/L, 230 mOsmol/L, 240 mOsmol/L, 250 mOsmol/L, 260 mOsmol/L, 270 mOsmol/L, 280 mOsmol/L, 290 mOsmol/L, 300 mOsmol/L, 310 mOsmol/L, 320 mOsmol/L, 330 mOsmol/L, 340 mOsmol/L, 350 mOsmol/L, 360 mOsmol/L, 370 mOsmol/L, 380 mOsmol/L, 390 mOsmol/L, or 400 mOsmol/L.

Examples of tonicity agents that may be used in the formulations provided herein include, without limitation, sodium chloride, dextrose, sucrose, xylitol, fructose, glycerol, sorbitol, mannitol, trehalose, potassium chloride, mannose, calcium chloride, magnesium chloride, other inorganic salts, other sugars, other sugar alcohols, and combinations thereof. In certain embodiments, an ADAMTS13 formulation may comprise at least one tonicity agent, or at least two, three, four, five, or more tonicity agents. In some embodiments, the ADAMTS13 formulations provided herein may further comprise one or more pharmaceutically acceptable excipients, carriers, and/or diluents. In certain embodiments, the ADAMTS13 compositions, including compositions with ADAMTS13, provided herein will have a tonicity in a range as described in U.S. Patent Application Publication No. 2011/0229455 and/or in U.S. Patent Application Publication No. 2014/0271611, each of which are incorporated herein by reference in their entirety for all purposes.

In addition, the formulations provided herein may further comprise other medicinal agents, carriers, adjuvants, diluents, tissue permeation enhancers, solubilizers, and the like. Methods for preparing compositions and formulations for pharmaceutical administration are known to those skilled in the art. See e.g., REMINGTON'S PHARMACEUTICAL SCIENCES, 18TH ED., Mack Publishing Co., Easton, Pa. (1990).

In certain embodiments, the ADAMTS13 formulation comprises a human recombinant ADAMTS13 (hrADAMTS13 or hrA13) protein, which can be obtained, for example, according to the methods set forth herein. In certain embodiments, the amino acid sequence is that of GenBank accession number NP_620594. In other embodiments, the amino acid sequence comprises amino acids 75 to 1427 of NP_620594, a natural or conservative variant thereof, or a biologically active fragment thereof. In certain embodiments, the hrADAMTS13 protein includes or incorporates a mutation or variant, for example a “missense” mutation or variant. For example, one suitable variant comprises a Q⁹⁷R mutation. In certain embodiments, the formulation or pharmaceutical composition comprises a combination of such rhADAMTS13 proteins, for example a mixture comprising a wild-type sequence and one or more variants, or a mixture comprising two or more variants. In certain embodiments comprising a mixture, one of the proteins may predominate, for example, a variant may predominate over a wild-type.

In one embodiment, the ADAMTS13 formulation comprises a liquid composition, suitable for lyophilization, and reconstituted for use, for example as set forth in U.S. Pat. No. 10,238,720. In certain embodiments, the compositions are liquid or lyophilized compositions. In other embodiments, a lyophilized composition is lyophilized from a liquid composition as described in U.S. Patent Application Publication No. 2011/0229455 and/or in U.S. Patent Application Publication No. 2014/0271611, each of which are incorporated herein by reference in their entirety and for all purposes.

The ADAMTS13 formulations provided herein may be formulated for administration via known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the ADAMTS13 formulations provided herein can be administered either systemically or locally. Systemic administration includes, without limitation: oral, subdermal, intraperitoneal, subcutaneous, transnasal, sublingual, or rectal routes of administration. Local administration includes, without limitation: topical, subcutaneous, intramuscular, and intraperitoneal routes of administration. In certain embodiments, intravenous administration is preferred. In certain embodiments, subcutaneous administration is preferred.

The present compositions and methods will be further illustrated in the following examples, without any limitation thereto.

G. Mixtures of ADAMTS13 Proteins

In certain embodiments, the pharmaceutical composition comprises a combination of at least one ADAMTS13 variant and ADAMTS13 protein (e.g., wildtype). In certain embodiments, the relative abundance (e.g., percentage) of ADAMTS13 variant present in the total amount of ADAMTS13 in the composition (i.e., including all ADAMTS13 variant(s) and wildtype) is between about 5% to about 95%, about 10% to about 90%, about 15% to about 85%, about 20% to about 80%, about 25% to about 75%, about 30% to about 70%, about 35% to about 65%, about 40% to about 60%, to about 45% to about 55%. In certain embodiments, the percentage of ADAMTS13 variant present in the total amount of ADAMTS13 in the composition is between about 40% to about 90%, about 40% to about 80%, about 45% to about 75%, about 50% to about 80%, about 50% to about 70%, or about 55% to about 65%. In certain embodiments, the percentage of ADAMTS13 variant present in the total amount of ADAMTS13 in the composition is between about 50% to about 75%, about 52% to about 72%, about 55% to about 70%, about 59% to about 72%. In certain embodiments, the percentage of ADAMTS13 variant present in the total amount of ADAMTS13 in the composition is between about 45% to about 85% or about 47% to about 84%. In certain embodiments, the percentage of ADAMTS13 variant present in the total amount of ADAMTS13 in the composition is between about 47% to about 84%. In certain embodiments, the percentage of ADAMTS13 variant present in the total amount of ADAMTS13 in the composition is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In certain embodiments, the percentage of ADAMTS13 variant present in the total amount of ADAMTS13 in the composition is about 52%, about 60%, about 61%, about 62%, about 63%, about 64%, about 65%, about 66%, or about 72%. In certain embodiments, the percentage of ADAMTS13 variant present in the total amount of ADAMTS13 in the composition is about 52%, about 65%, or about 72%.

In certain embodiments, the pharmaceutical composition comprises a combination of at least one ADAMTS13 variant and ADAMTS13 protein (e.g., wildtype). In certain embodiments, the ratio of ADAMTS13 variant to ADAMTS13 protein is about 4:1 to about 1:4, about 3:1 to about 1:3, about 2:1 to about 1:2. In certain embodiments, the ratio of ADAMTS13 variant to ADAMTS13 protein is about 3:1 to about 1:3, about 2:1 to about 1:2, or about 2:1 to about 1:3, or about 1:1 to about 1:3, or about 1:1.1 to about 1:2.9, or about 1:1.2 to about 1:2.8, or about 1:1.3 to about 1:2.7, or about 1:1.4 to about 1:2.6, or about 1:1.5 to about 1:2.5, or about 1:1.6 to about 1:2.4, or about 1:1.7 to about 1:2.3, or about 1:1.8 to about 1:2.2, or about 1:1.9 to about 1:2.1. In certain embodiments, the ratio of ADAMTS13 variant to ADAMTS13 protein is about 1.1:1 to about 2.9:1, or about 1.2:1 to about 2.8:1, or about 1.3:1 to about 2.7:1, or about 1.4:1 to about 2.6:1, or about 1.5:1 to about 2.5:1, or about 1.6:1 to about 2.4:1, or about 1.7:1 to about 2.3:1, or about 1.8:1 to about 2.2:1, or about 1.9:1 to about 2.1:1. In certain embodiments, the ratio of ADAMTS13 variant to ADAMTS13 wildtype is about 1:1 to about 1:3. In certain embodiments, the ratio of ADAMTS13 variant to ADAMTS13 wildtype is about 3:1 to about 1:1. In certain embodiments, the ratio of ADAMTS13 variant to ADAMTS13 wildtype is about 1:1.1 to about 1:2.5. In certain embodiments, the ratio of ADAMTS13 variant to ADAMTS13 wildtype is about 4:1, about 4:1.5, about 4:2, about 4:2.5, about 4:3, about 4:3.5, about 3:1, about 3:1.5, about 3:2, about 3:2.5, about 2:1, or about 2:1.5. In certain embodiments, the ratio of ADAMTS13 variant to ADAMTS13 wildtype is about 1:1.5, about 1:2, about 1:2.5, about 1:3, about 1:3.5, about 1:4, about 2:2.5, about 2:3, about 2:3.5, about 2:4, about 3:3.5, or about 3:4. In certain embodiments, the ratio of ADAMTS13 variant to ADAMTS13 wildtype is about 1:3. In certain embodiments, the ratio of ADAMTS13 variant to ADAMTS13 wildtype is about 3:1. In certain embodiments, the ratio of ADAMTS13 variant to ADAMTS13 wildtype is about 2:1. In certain embodiments, the ratio of ADAMTS13 variant to ADAMTS13 wildtype is about 1:2. In certain embodiments, the ratio of ADAMTS13 variant to ADAMTS13 wildtype is about 1:1. In certain embodiments, the ratio of ADAMTS13 variant to ADAMTS13 wildtype is about 3:2.

In certain embodiments, the relative abundance, percentage, and/or ratio is determined by a peptide mapping method. In certain embodiments, the relative abundance, percentage, and/or ratio is determined by a peptide mapping method as described in Example 3. In certain embodiments, the relative abundance, percentage, and/or ratio is determined by HPLC analysis of tryptic peptides separated by liquid chromatography followed by mass spectrometry analysis. In certain embodiments, the relative abundance, percentage, and/or ratio is based on intensities in extracted ion chromatograms. In certain embodiments, the relative abundance, percentage, and/or ratio is determined based on the peak area of tryptic peptides of the ADAMTS13 variant (e.g., Q⁹⁷R ADAMTS13 variant) in relation to the sum of the peak areas of all ADAMTS13 proteins and variants in the composition (e.g., sum total of Q⁹⁷R ADAMTS13 variant and Q⁹⁷ ADAMTS13 protein). In certain embodiments, the tryptic peptides of all ADAMTS13 proteins and variants in the composition being measured are specific to the at least one amino acid difference between the ADAMTS13 variant as compared to all other ADAMTS13 proteins and variants in the composition. For example, the tryptic peptide(s) that can be measured for the Q⁹⁷R ADAMTS13 variant can be AAGGILHLELLVAVGPDVFQAHR or a combination of AAGGILHLELLVAVGPDVFQAHR and EDTER and the tryptic peptide measured for the Q⁹⁷ ADAMTS13 protein can be AAGGILHLELLVAVGPDVFQAHQEDTER.

In certain embodiments, the relative abundance, percentage, and/or ratio is determined based on total weight of ADAMTS13 variant in relation to the sum total weight of all ADAMTS13 proteins and variants in the composition.

H. Additional Compositions and Pharmaceutical Excipients

Compositions or pharmaceutical compositions useful in the compounds and methods of the disclosure containing at least one ADAMTS13 as an active ingredient contain, in various aspects, pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form.

A variety of aqueous carriers, e.g., water, buffered water, 0.4% saline, 0.3% glycine, or aqueous suspensions contain, in various aspects, the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents, in some instances, are a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions, in certain embodiments, contain one or more preservatives, for example ethyl, or n-propyl, p-hydroxybenzoate.

In certain embodiments, the ADAMTS13 compositions, provided herein may further comprise one or more pharmaceutically acceptable excipients, carriers, and/or diluents as described in U.S. Patent Application No. 20110229455 and/or in U.S. Patent Application Publication No. 2014/0271611, each of which are incorporated herein by reference in their entirety for all purposes.

### Exemplary Embodiments

1. A method of treating or preventing at least one condition or complication in a subject infected with severe acute respiratory syndrome coronavirus 2 (SARS-CoV-2) or suffering from coronavirus disease 2019 (COVID-19), the method comprising administering to the subject in need thereof a therapeutically effective amount of a composition comprising A disintegrin and metalloproteinase with a thrombospondin type 1 motif (ADAMTS13).
2. A method of treating a subject at risk of developing at least one condition or complication associated with severe acute respiratory syndrome coronavirus 2 (SARS-CoV-2) infection or coronavirus disease 2019 (COVID-19), the method comprising administering to the subject in need thereof a therapeutically effective amount of a composition comprising A disintegrin and metalloproteinase with a thrombospondin type 1 motif (ADAMTS13).
3. A method of treating or preventing at least one condition or complication in a subject infected with severe acute respiratory syndrome coronavirus 2 (SARS-CoV-2) or suffering from coronavirus disease 2019 (COVID-19), comprising the steps of:
   a) administering to the subject in need thereof a therapeutically effective amount of a pharmaceutical composition comprising a recombinant A Disintegrin And Metalloproteinase with Thrombospondin type 1 motif, member-13 (ADAMTS13), wherein said therapeutically effective amount is sufficient to:
      i) reduce circulating ultra-high molecular weight (UHMW) von Willebrand factor (VWF) multimers to a level that is at least about 5%, at least about 10%, or at least about 20% decreased compared to a measured level of VWF in the subject's blood prior to administration;
      ii) reduce circulating UHMW VWF multimers to a level that is no more than about 5%, no more than about 10%, or no more than about 20% above a normal VWF baseline value;
      iii) reduce circulating VWF to a level that is at least about 5%, at least about 10%, or at least about 20% decreased compared to a measured level of VWF in the subject's blood prior to administration;
      iv) reduce circulating VWF to a level that is no more than about 5%, no more than about 10%, or no more than about 20% above a normal VWF baseline value;
      v) reduce VWF activity level to a level that is at least about 5%, at least about 10%, or at least about 20% decreased compared to a measured level of VWF activity in the subject's blood prior to administration;
      vi) reduce VWF activity level to a level that is no more than about 5%, no more than about 10%, or no more than about 20% above a normal VWF activity baseline value;
      vii) increase circulating ADAMTS13 levels from about 100% to about 150% above a normal ADAMTS13 baseline value; or
      viii) combinations of i)-vii); and
   b) periodically monitoring and adjusting the administered amount to maintain said reduced level of circulating VWF, UHMW VWF multimers, or combinations thereof
4. The method of any one of embodiments 1-3, wherein the subject is administered the composition comprising ADAMTS13 before the condition or complication is present.
5. The method of any one of embodiments 1-4, wherein the subject is administered the composition comprising ADAMTS13 after the condition or complication is present.
6. The method of any one of embodiments 1-5, wherein the condition or complication is a coagulopathy, blood-clotting disorder, infarction, thrombosis, embolism, stroke, veno-occlusive disorders, prothrombotic conditions, sepsis, renal failure, respiratory failure, acute respiratory distress syndrome (ARDS), chronic obstructive pulmonary disease (COPD), thrombotic microangiopathy (TMA), pneumonia, asthma, hypertension, elevated plasma levels of VWF and/or its multimers (especially ultralarge multimers (UHMW)), elevated plasma VWF activity levels, reduced plasma levels of endogenous ADAMTS13, inflammation, elevated cytokine levels, or combination thereof
7. The method of embodiment 6, wherein the thrombosis is deep vein thrombosis (DVT).
8. The method of embodiment 6, wherein the embolism is a pulmonary embolism (PE).
9. The method of embodiment 6, wherein the complication is elevated plasma levels of VWF, elevated plasma levels of UHMW VWF multimers, and/or reduced plasma levels of endogenous ADAMTS13.
10. The method of embodiment 6, wherein the complication is an elevated cytokine level.
11. The method of embodiment 6, wherein the COVID-19 complication is ARDS, COPD, TMA, pneumonia, asthma, pulmonary hypertension, deep vein thromboses, pulmonary embolism, or combinations thereof
12. The method of any one of embodiments 1-11, wherein the subject is 65 years of age or older.
13. The method of any one of embodiments 1-12, wherein the subject presents with a risk factor.
14. The method of embodiment 13, wherein the risk factor is elevated plasma levels of VWF, elevated plasma levels of ultralarge multimers (UHMW) VWF multimers, elevated plasma VWF activity levels, reduced plasma levels of endogenous ADAMTS13, reduced activity of endogenous ADAMTS13 activity, elevated cytokine levels, coagulopathies, blood-clotting disorders, veno-occlusive disorders, prothrombotic conditions, inherited thrombotic thrombocytopenic purpura (TTP), acquired TTP, disseminated intravascular coagulation (DIC), sepsis, sickle cell, respiratory failure, acute respiratory distress syndrome (ARDS), chronic obstructive pulmonary disease (COPD), thrombotic microangiopathy (TMA), pneumonia, asthma, pregnancy, menopause, pen-menopause, hypertension, pulmonary hypertension, thromboses, embolism, myocardial infarction, stroke, cough, shortness of breath, pulmonary infiltrates, respiratory failure, elevated plasma fibrogen, activated hemostasis pathway, intensive care unit (ICU) admission, or combinations thereof.
15. The method of embodiment 14, wherein the risk factor is elevated plasma levels of VWF, elevated plasma levels of UHMW VWF multimers, and/or a reduced plasma level of endogenous ADAMTS13.
16. The method of embodiment 14, wherein the risk factor is an elevated cytokine level.
17. The method of embodiment 14, wherein the risk factor is a prothrombotic condition.
18. The method of embodiment 14, wherein the risk factor is one of ARDS, COPD, TMA, pneumonia, asthma, pregnancy, menopause, peri-menopause, hypertension, pulmonary hypertension, deep vein thromboses, or pulmonary embolism.
19. The method of any one of embodiments 1-18, wherein administering the composition comprising ADAMTS13 to the subject reduces the duration, severity, or frequency of occurrence of the condition or complication compared to a subject that was not administered the composition comprising ADAMTS13.
20. The method of any one of embodiments 1-19, wherein administering the composition comprising ADAMTS13 to the subject reduces plasma level of VWF protein, plasma level of VWF multimers, VWF activity, plasma ratio of VWF to ADAMTS13 (VWF:A13), platelet aggregation, blood clotting, thrombosis, embolism, infarction, veno-occlusion, stroke, inflammation, plasma cytokine levels, or combinations thereof as compared to a normal baseline range in a healthy individual.
21. The method of embodiment 20, wherein the VWF multimer is an UHMW multimer.
22. The method of embodiment 20, wherein administering the composition comprising ADAMTS13 to the subject reduces plasma level of VWF protein, plasma level of VWF multimers, VWF activity, plasma VWF:A13, or combinations thereof 23. The method of embodiment 20, wherein administering the composition comprising
ADAMTS13 to the subject reduces platelet aggregation, blood clotting, thrombosis, embolism, infarction, veno-occlusion, stroke, or combinations thereof
24. The method of any one of embodiments 1-23, wherein administering the composition comprising ADAMTS13 to the subject increases plasma levels of ADAMTS13, plasma ADAMTS13 activity, or combinations thereof to a normal baseline range in a healthy individual.
25. The method of any one of embodiments 1-23, wherein administering the composition comprising ADAMTS13 to the subject increases plasma level of ADAMTS13, plasma ADAMTS13 activity, or combinations thereof from about 20-100%, above a normal baseline range or normal baseline value of ADAMTS13 plasma level or ADAMTS13 activity level.
26. The method of any one of embodiments 1-23, wherein administering the composition comprising ADAMTS13 to the subject increases plasma level of ADAMTS13, plasma ADAMTS13 activity, or combinations thereof from about 100-150%, compared to a normal baseline value of ADAMTS13 plasma level or ADAMTS13 activity level.
27. The method of any one of embodiments 1-26, wherein the therapeutically effective amount of the ADAMTS13 is about 10-400 IU/kg for a subject having a VWF level that is more than about 5% higher than a baseline corresponding to the upper limit of a predetermined normal range of VWF levels in healthy subjects.
28. The method of any one of embodiments 1-26, wherein the therapeutically effective amount of the ADAMTS13 is about 10-400 IU/kg or about 40-400 IU/kg for a subject having a VWF level that is at least about two times higher than a normal baseline VWF level in healthy subjects.
29. The method of any one of embodiments 1-26, wherein the therapeutically effective amount of the ADAMTS13 is about 10-400 IU/kg or about 40-400 IU/kg for a subject having a VWF level that is at least about three times higher than a normal baseline VWF level in healthy subjects.
30. The method of any one of embodiments 1-26, wherein the therapeutically effective amount of the ADAMTS13 is about 10-400 IU/kg for a subject having an ADAMTS13 activity and/or level between about 30-70% that of a normal ADAMTS13 baseline activity and/or level in healthy subjects.
31. The method of any one of embodiments 1-26, wherein the therapeutically effective amount of the ADAMTS13 is about 10-400 IU/kg or about 40-400 IU/kg for a subject having an ADAMTS13 activity and/or level less than about 20% of a normal ADAMTS13 baseline activity and/or level in healthy subjects.
32. The method of any one of embodiments 1-26, wherein the therapeutically effective amount of the ADAMTS13 is about 10-400 IU/kg or about 40-400 IU/kg for a subject having a ultra-high molecular weight (UHMW) VWF multimer level between about 100-130% that of a normal UHMW VWF multimer baseline level in healthy subjects.
33. The method of any one of embodiments 1-26, wherein the therapeutically effective amount of the ADAMTS13 is about 10-400 IU/kg for a subject having a ultra-high molecular weight (UHMW) VWF multimer level at least about 101%, at least about 105%, or at least about 107% that of a normal UHMW VWF multimer baseline level in healthy subjects.
34. The method of any one of embodiments 1-33, wherein the therapeutically effective amount of the ADAMTS13 is about 10-320 IU/kg, about 10-300 IU/kg, about 10-200 IU/kg, about 10-180 IU/kg, about 10-160 IU/kg, about 10-80 IU/kg, about 10-60 IU/kg, about 10-40 IU/kg, about 10-20 IU/kg, about 20-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, or about 20-30 IU/kg.
35. The method of any one of embodiments 1-33, wherein the therapeutically effective amount of the ADAMTS13 is about 30-320 IU/kg, about 30-300 IU/kg, about 30-180 IU/kg, about 30-160 IU/kg, about 30-60 IU/kg, about 40-400 IU/kg, about 40-320 IU/kg, about 40-300 IU/kg, about 40-180 IU/kg, about 40-160 IU/kg, about 40-80 IU/kg or about 40-60 IU/kg.
36. The method of any one of embodiments 1-33, wherein the therapeutically effective amount of the ADAMTS13 is about 10-60 IU/kg.
37. The method of any one of embodiments 1-33, wherein the therapeutically effective amount of the ADAMTS13 is about 10-40 IU/kg.
38. The method of any one of embodiments 1-33, wherein the therapeutically effective amount of the ADAMTS13 is about 10-20 IU/kg.
39. The method of any one of embodiments 1-33, wherein the therapeutically effective amount of the ADAMTS13 is about 40-320 IU/kg.
40. The method of any one of embodiments 1-33, wherein the therapeutically effective amount of the ADAMTS13 is about 40-160 IU/kg.
41. The method of any one of embodiments 1-33, wherein the therapeutically effective amount of the ADAMTS13 is about 40-80 IU/kg
42. The method of any one of embodiments 1-33, wherein the therapeutically effective amount of the ADAMTS13 is about 40-60 IU/kg.
43. The method of any one of embodiments 1-42, wherein the composition comprising ADAMTS13 is administered monthly, every two weeks, weekly, twice a week, three times a week, every other day, daily, every 12 hours, every 8 hours, every six hours, every four hours, every two hours, or every hour.
44. The method of any one of embodiments 1-42, wherein the composition comprising ADAMTS13 is administered once daily or twice-daily.
45. The method of any one of embodiments 1-42, wherein the composition comprising ADAMTS13 is administered within 24 hours of measuring the subject's level and/or activity of VWF protein and/or measuring the subject's level and/or activity of ADAMTS13.
46. The method of any one of embodiments 1-45, wherein the composition comprising ADAMTS13 is administered intravenously.
47. The method of any one of embodiments 1-45, wherein the composition comprising ADAMTS13 is administered subcutaneously.
48. The method of any one of embodiments 1-45, wherein said administration comprises delivering an intravenous bolus of the composition comprising ADAMTS13.
49. The method of any one of embodiments 1-45, wherein said administration comprises delivering an intravenous infusion of the composition comprising ADAMTS13.
50. The method of any one of embodiments 1-49, wherein the composition comprising ADAMTS13 comprises plasma derived human ADAMTS13.
51. The method of any one of embodiments 1-49, wherein the composition comprising ADAMTS13 comprises recombinant ADAMTS13.
52. The method of embodiment 51, wherein the recombinant ADAMTS13 is a human ADAMTS13.
53. The method of any one of embodiments 1-49, 51, or 52, wherein the ADAMTS13 is a mixture of ADAMTS13 variant and wildtype ADAMTS13.
54. The method of embodiment 53, wherein the ratio of ADAMTS13 variant to wildtype ADAMTS13 is about 1:1 to about 3:1, about 1:1 or about 3:2.
55. The method of embodiment 53, wherein the ADAMTS13 variant constitutes between about 52% to about 72% or between about 47% to about 84% of total ADAMTS13 in the composition.
56. The method of embodiment 54 or embodiment 55, wherein the ratio or percentage is determined by a) peptide mapping, b) HPLC analysis of tryptic peptides separated by liquid chromatography followed by mass spectrometry analysis, or c) based on intensities in extracted ion chromatograms.
57. The method of any one of embodiments 51-56, wherein the recombinant ADAMTS13 or wildtype ADAMTS13 comprises the amino acid sequence of SEQ ID NO: 1, or an amino acid sequence having at least 80% sequence identity thereof
58. The method of embodiment 57, wherein the recombinant ADAMTS13 or wildtype ADAMTS13 consists essentially of the amino acid sequence of SEQ ID NO: 1.
59. The method of embodiment 57, wherein the recombinant ADAMTS13 or wildtype ADAMTS13 consists of the amino acid sequence of SEQ ID NO: 1.
60. The method of any one of embodiments 51-59, wherein the recombinant ADAMTS13 or ADAMTS13 variant is an ADAMTS13 variant comprising a single amino acid substitution at amino acid Q97 as denoted in SEQ ID NO: 1, or the equivalent amino acid position in an ADAMTS13.
61. The method of embodiment 60, wherein the single amino acid change is from a Q to a D, E, K, H, L, N, P, or R.
62. The method of embodiment 60, wherein the single amino acid change is from a Q to an R.
63. The method of embodiment 60, wherein the ADAMTS13 variant comprises the amino acid sequence of SEQ ID NO: 2, or an amino acid sequence having at least 80% sequence identity thereof
64. The method of embodiment 60, wherein the ADAMTS13 variant consists essentially of the amino acid sequence of SEQ ID NO: 2.
65. The method of embodiment 60, wherein the ADAMTS13 variant consists of the amino acid sequence of SEQ ID NO: 2.
66. The method of any one of embodiments 1-65, wherein the composition comprises an aqueous solution that is reconstituted from a lyophilized formulation.
67. The method of embodiment 66, wherein the lyophilized formulation is lyophilized from a liquid formulation comprising at least about 200 FRET-U/ml of the ADAMTS13.
68. The method of embodiment 67, wherein the pH of the liquid formulation is about 7.0-7.5.
69. The method of embodiment 67, wherein the liquid formulation that is lyophilized further comprises at least one of histidine, sodium chloride, sucrose, trehalose, mannitol, calcium chloride, Polysorbate 80, and combinations thereof
70. The method of embodiment 69, wherein the liquid formulation that is lyophilized further comprises about 20 mM histidine, about 30-60 mM sodium chloride, about 1 2% sucrose, about 3% mannitol, about 4 mM calcium chloride, and about 0.05% Polysorbate 80.
71. The method of any one of embodiments 1-70, comprising the further steps of:
   periodically measuring the subject's VWF level; and
   reducing the therapeutically effective amount to about 10-100 IU/kg when the subject's VWF level is within a predetermined baseline range of a healthy individual.
72. The method of any one of embodiments 20-71, wherein a normal baseline range for VWF levels is a range of about 50-200% or about 42-136% of an established or predetermined average baseline.
73. The method of any one of embodiments 20-72, wherein a normal baseline range for ADAMTS13 levels is a range of about 40-160% or about 87-113% of an established or predetermined average baseline.
74. A method of determining whether a subject diagnosed with COVID-19 is at an increased risk for a thrombotic coagulopathy, said method comprising the steps of:
   a) measuring in a blood plasma sample one or more of:
      i) a plasma level of VWF protein;
      ii) an activity level of VWF in the plasma sample;
      iii) a plasma level of UHMW VWF protein multimers;
      iv) a plasma level of ADAMTS13 protein; or
      v) an activity level of ADAMTS13 protein in the plasma sample; and
   b) comparing the plasma level(s) or activity level(s) measured in step a) to a baseline range or baseline value for the same plasma level(s) or activity level(s); and
   c) identifying the subject being at risk for a thrombotic coagulopathy when at least one of the following is met:
      i) the plasma level of VWF protein is increased;
      ii) the activity level of VWF is increased;
      iii) plasma UHMW VWF protein multimers are detected or the plasma level of UHMW VWF protein multimers is increased;
      iv) the plasma level of ADAMTS13 protein is decreased; or
      v) the activity level of ADAMTS13 protein is decreased, as compared to the baseline range or baseline value for the same plasma level(s) or activity level(s).
75. The method of embodiment 74, wherein at least the plasma level of VWF protein is increased.
76. The method of 74 or 75, wherein at least the activity level of VWF is increased.
77. The method of any one of embodiments 74-76, wherein at least UHMW VWF protein multimers are detected or the plasma level of UHMW VWF protein multimers is increased.
78. The method of any one of embodiments 74-77, wherein at least the plasma level of ADAMTS13 protein is decreased.
79. The method of any one of embodiments 74-78, wherein at least the activity level of ADAMTS13 protein is decreased.
80. The method of any one of embodiments 74-79, wherein thrombotic coagulopathy is platelet aggregation, blood clotting, a thrombosis, a thrombotic microangiopathy, an embolism, an infarction, veno-occlusion, a stroke, renal failure resulting from thrombosis, or combinations thereof
81. The method of embodiment 80, wherein the thrombosis is deep vein thrombosis (DVT).
82. The method of embodiment 80, wherein the embolism is a pulmonary embolism (PE).
83. The method of embodiment 80, wherein the thrombotic coagulopathy is renal failure resulting from thrombosis.
84. The method of any one of embodiments 74-83, wherein the subject is at risk for developing a thrombotic coagulopathy when the plasma level of VWF protein is about 120% to about 300% of the baseline value for said VWF protein plasma level.
85. The method of any one of embodiments 74-84, wherein the subject is at risk for developing a thrombotic coagulopathy when the plasma level of VWF protein is about 300% or more of the baseline value for said VWF protein plasma level.
86. The method of any one of embodiments 74-85, wherein the subject is at risk for developing a thrombotic coagulopathy when the activity level of VWF in the plasma sample is about 120% to about 300% of the baseline value for said VWF activity level.
87. The method of any one of embodiments 74-86, wherein the subject is at risk for developing a thrombotic coagulopathy when the activity level of VWF in the plasma sample is about 300% or more of the baseline value for said VWF activity level.
88. The method of any one of embodiments 74-87, wherein the subject is at risk for developing a thrombotic coagulopathy when the plasma level of ADAMTS13 protein is about 70% to about 100% of the baseline value for said ADAMTS13 protein plasma level.
89. The method of any one of embodiments 74-88, wherein the subject is at risk for developing a thrombotic coagulopathy when the plasma level of ADAMTS13 protein is 70% or less of the baseline value for said ADAMTS13 protein plasma level.
90. The method of any one of embodiments 74-89, wherein the subject is at risk for developing a thrombotic coagulopathy when the activity level of ADAMTS13 in the plasma sample is about 70% to about 100% of the baseline value for said ADAMTS13 activity level.
91. The method of any one of embodiments 74-90, wherein the subject is at risk for developing a thrombotic coagulopathy when the activity level of ADAMTS13 in the plasma sample is 70% or less of the baseline value for said ADAMTS13 activity level.
92. The method of any one of embodiments 74-91, wherein the subject is at risk for developing a thrombotic coagulopathy when the plasma level of UHMW VWF multimers is about 100% to about 110% of the baseline value for said UHMW VWF multimer plasma level.
93. The method of any one of embodiments 74-92, wherein the subject is at risk for developing a thrombotic coagulopathy when the plasma level of UHMW VWF multimers is 110% or more of the baseline value for said UHMW VWF multimer plasma level.
94. The method of any one of embodiments 74-93, wherein the subject is at risk for developing a thrombotic coagulopathy when the ratio of VWF:A13 levels in the plasma sample is 3 or less.
95. The method of any one of embodiments 74-94, wherein the subject is at risk for developing a thrombotic coagulopathy when the ratio of VWF:A13 levels in the plasma sample is greater than 3.
96. The method of any one of embodiments 74-95, wherein the baseline value is a predetermine value based on a normal control population.
97. The method any one of embodiments 74-96, wherein the baseline value is a mean of a predetermine range of a normal control population.
98. A method of determining whether a subject diagnosed with COVID-19 is at risk for a thrombotic coagulopathy, said method comprising the steps of:
   a) measuring in a blood plasma sample one or more of:
      i) a plasma level of VWF protein;
      ii) an activity level of VWF in the plasma sample;
      iii) a plasma level of UHMW VWF protein multimers;
      iv) a plasma level of ADAMTS13 protein; and/or
      v) an activity level of ADAMTS13 protein in the plasma sample; and
   b) identifying the subject being at risk for a thrombotic coagulopathy when at least one of the following is met:
      i) the plasma level of VWF protein is at least about 1.2 IU/ml;
      ii) the VWF activity level is at least about 1.2 IU/ml or about 1.8 IU/ml;
      iii) plasma UHMW VWF protein multimers are detected;
      iv) the plasma level of ADAMTS13 protein is no more than about 0.7 IU/ml; and/or
      v) the activity level of ADAMTS13 protein is no more than about 0.8 or about 0.9 IU/ml.
99. The method of embodiment 98, wherein in step b) the subject is at a high risk for a thrombotic coagulopathy when at least one of the following is met:
   i) the plasma level of VWF protein is at least about 4.5 IU/ml;
   ii) the VWF activity level is at least about 3.3 IU/ml or about 4.4 IU/ml;
   iii) the plasma level of ADAMTS13 protein is no more than about 0.4 IU/ml; and/or
   iv) the activity level of ADAMTS13 protein is no more than about 0.4 or about 0.5 IU/ml.
100. The method of any one of embodiments 74-99, wherein the VWF activity level is measured by VWF ristocetin co-factor activity.
101. The method of any one of embodiments 74-99, wherein the VWF activity level is measured by VWF collagen binding activity.
102. The method of any one of embodiments 74-99, wherein the ADAMTS13 activity level is measured by ELISA.
103. The method of any one of embodiments 74-99, wherein the VWF activity level is measured by FRETS.
104. The method of any one of embodiments 74-103, wherein the subject is diagnosed with COVID by detecting SARS-CoV-2 RNA by PCR from a blood or nasal mucus sample taken from the subject.
105. The method of any one of embodiments 74-104, wherein the subject is diagnosed with COVID by SARS-CoV-2 seroconversion.
106. The method of any one of embodiments 74-105, wherein the subject is diagnosed with COVID by detection of SARS-CoV-2 antibodies in the subject's plasma.
107. The method of any one of embodiments 74-106, further comprising obtaining a blood sample from the subject.
108. The method of embodiment 107, wherein the blood sample is treated with an anticoagulant.
109. The method of embodiment 108, wherein the anticoagulant is EDTA, sodium citrate, or heparin.
110. The method of any one of embodiments 74-109, wherein the method further comprises administering to the subject a composition comprising a therapeutically effective amount of ADAMTS13.
111. The method of embodiment 110, wherein the therapeutically effective amount of the ADAMTS13 is about 10-400 IU/kg.
112. The method of embodiment 110 or embodiment 111, wherein the therapeutically effective amount of the ADAMTS13 is about 10-320 IU/kg, about 10-300 IU/kg, about 10-200 IU/kg, about 10-180 IU/kg, about 10-160 IU/kg, about 10-80 IU/kg, about 10-60 IU/kg, about 10-40 IU/kg, about 10-20 IU/kg, about 20-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, or about 20-30 IU/kg.
113. The method of embodiment 110 or embodiment 111, wherein the therapeutically effective amount of the ADAMTS13 is about 30-320 IU/kg, about 30-300 IU/kg, about 30-180 IU/kg, about 30-160 IU/kg, about 30-60 IU/kg, about 40-400 IU/kg, about 40-320 IU/kg, about 40-300 IU/kg, about 40-180 IU/kg, about 40-160 IU/kg, about 40-80 IU/kg or about 40-60 IU/kg.
114. The method of any one of embodiments 84, 86, 88, 90, 92, 94, or 98, wherein the method further comprises administering to the subject a composition comprising a therapeutically effective amount of ADAMTS13 and wherein the therapeutically effective amount of the ADAMTS13 is about 10-40 IU/kg, about 10-30 IU/kg, about 10-20 IU/kg, about 20-40 IU/kg, or about 20-30 IU/kg.
115. The method of embodiment 114, wherein the therapeutically effective amount of the ADAMTS13 is about 10 IU/kg, about 20 IU/kg, about 30 IU/kg, or about 40 IU/kg.
116. The method of any one of embodiments 85, 87, 89, 91, 93, 95, or 99, wherein the method further comprises administering to the subject a composition comprising a therapeutically effective amount of ADAMTS13 and wherein the therapeutically effective amount of the ADAMTS13 is about 40-400 IU/kg, about 40-320 IU/kg, about 40-300 IU/kg, about 40-180 IU/kg, about 40-160 IU/kg, about 40-80 IU/kg or about 40-60 IU/kg.
117. The method of embodiment 116, wherein the therapeutically effective amount of the ADAMTS13 is about 40 IU/kg, about 60 IU/kg, about 80 IU/kg, or about 160 IU/kg
118. A kit for determining whether a subject diagnosed with COVID-19 is at risk for a thrombotic coagulopathy, said kit comprising (i) one or more reagents for determining one or more of the plasma level of VWF protein, activity level of VWF, plasma level of UHMW VWF multimers, plasma level of ADAMTS13 protein, activity level of ADAMTS13, (ii) optionally packaging and/or instructions for use, and (iii) optionally one or more reagents for detecting SARS-CoV-2 or diagnosing COVID-19.

### EXAMPLES

### Example 1: Expression of Recombinant ADAMTS13 (rA13)

A chemostat cell-culture of the recombinant CHO cell line expressing human ADAMTS13, was grown in chemically defined BACD-A13 medium supplemented with additional zinc and vitamin B3. The 10 L culture was maintained for 53 days and rA13 protein and activity production was monitored over time.

Recombinant CHO cells expressing human ADAMTS13 were adapted to a chemically defined proprietary medium (BCS medium). A DWCB was thawed and cell inoculum was prepared in BCS medium. Cells propagated from the rA13 expression clone #640-2 were transferred to a 10 L bioreactor with Rushton type impellers and cultivated in repeated batch cultures with proprietary BACD-A13 medium under an inline controlled pH of 7.15-7.20 at 37° C. with a dissolved oxygen concentration of 20% air saturation. After 2 batch cultures were grown to the final working volume of 10 L, the bioreactor was switched to continuous medium feed on day 5 and operated for an additional 48 days in a chemostat mode.

Samples of the supernatant from the bioreactors were taken weekly and analyzed for rA13 protein production by ELISA and rA13 activity by FRETS-VWF73 assay. Cell counts were determined by Nucleocounter technology. Dilution rates were measured and used for calculation of growth rates and volumetric productivities.

Under continuous culture conditions using chemically defined BACD-A13 medium supplemented with zinc and nicotinamide at a final concentration of 1.432 mg/L ZnSO₄7H₂O and 7.02 mg/L nicotinamide, high levels of rA13 protein production, between 0.9 and 1.3 mg/L/D, and specific activities, between about 800 and 1100 mU/μg rA13, were achieved (Table 1). Notably, volumetric and cell specific productivities increased over time in the long term culture, likely due to increasing growth and dilution rates over time. The high specific activity of the expressed rA13 could be at least maintained at a constantly high level over at least entire 7 weeks the culture was grown under chemostatic conditions. In fact, the specific activity of the rA13 produced in the culture actually increased from about 800 mU/μg A13 at week 2 to about 1100 mU/μg A13 at week 7.

TABLE 1
Fermentation data for batch experiment
CP_07/18_M07: hA13 CHO Klon #985/1 985 DWCB#01.

| | | Specific | | | | | | |
|---|---|---|---|---|---|---|---|---|
| Chemostat | Cell | Growth | Dilution | A13 | A13 | Specific | FRETS | A13 |
| Culture | Concentration | Rate | Rate | FRETS | ELISA | Activity | Yield | Yield |
| Week No. | [10⁶ cells/ml] | [1/d] | [1/d] | [mU/ml] | [μg/ml] | [mU/μg] | [U/L/d] | [mg/L/d] |
| 2 | 1.43 | 0.36 | 0.36 | 1954 | 2.48 | 788 | 713 | 0.91 |
| 3 | 1.56 | 0.41 | 0.40 | 2254 | 2.32 | 972 | 913 | 0.94 |
| 4 | 1.46 | 0.38 | 0.40 | 2244 | 2.41 | 931 | 889 | 0.95 |
| 5 | 1.58 | 0.43 | 0.43 | 2514 | 2.88 | 873 | 1086 | 1.24 |
| 6 | 1.70 | 0.51 | 0.46 | 2737 | 2.71 | 1010 | 1270 | 1.26 |
| 7 | 1.76 | 0.53 | 0.52 | 2322 | 2.18 | 1065 | 1200 | 1.13 |

A suitable rADAMTS13 for expression according to this example is the human ADAMTS13 protein comprising the amino acid sequence of GenBank accession number NP_620594.

### Example 2: Expression and Purification of Recombinant Human ADAMTS13

Recombinant ADAMTS13 is generated by a recombinant Chinese Hamster Ovary (CHO) cell clone in a fermentation process in suspension culture. The growth medium, is both free of human or animal derived substances and recombinant proteins. Examples of these types of growth mediums useful for the expression of ADAMTS13 can be found, for example, in U.S. Pat. No. 8,313,926. The manufacturing process utilizes a continuous (chemostat) cell culture method. The purification process starts with an initial cell removal step by filtration. The cell free product of up to 4 subsequent days is combined to produce one downstream batch. The pooled, filtered harvests are concentrated by an ultra/diafiltration and then subjected to a solvent detergent virus inactivation step. Further purification includes a chromatographic capture step (Anion Exchange), a nanofiltration step (second virus reduction step), a negative chromatography step (hydroxyapatite) followed by a mixed mode chromatography (Capto MMC) and a final chromatographic concentration and pre-formulation step (Cation Exchange). The pre-formulated bulk drug substance (BDS) is frozen at −60° C. in a temperature-controlled freezer.

A suitable rADAMTS13 for expression according to this example is the human ADAMTS13 protein comprising the amino acid sequence of GenBank accession number NP_620594. Also suitable would be a “missense” variant of any such rADAMTS13 sequence, including for example a Q⁹⁷R variant. Combinations of any of these may also be used.

### Example 3: FRETS-VWF73 Assay for ADAMTS13 Activity

The proteolytic activity of ADAMTS13 would be measured against a fluorescence-quenching substrate (FRETS-VWF73, Peptides Institute, Inc; Osaka, Japan) according to the assay description of the manufacturer. Briefly, rADAMTS13 samples are diluted (in 100 μL total volume) in buffer containing 5 mM Bis-Tris, 25 mM CaCl₂, and 0.005% Tween20 and transferred into a black microtiter plate. Samples are measured against a reference curve of diluted human plasma samples (from 80 to 5 mU/mL plasma). The reaction is started by adding the substrate (100 μL, FRETS-VWF73; 2 μM final concentration) and fluorescence is measured every two minutes for 45 minutes in a fluorescence spectrophotometer with lex=360 nm and lem=460 at 30° C. (FLx800, Bio Tek). The activity results are read off a reference curve of human plasma. Data are expressed as Unit/mL. See also, Kokame et al., FRETS-VWF73, a first fluorogenic substrate for ADAMTS13 assay, Br J Haematol 2005; 129: 93-100. Samples are measured against a reference curve obtained with serial dilutions of pooled normal human plasma (George King Biomedical, Overland Park, Kans., USA), with an assigned ADAMTS13 activity concentration of 1 U mL-1. Normal human plasma was regarded as 1 Unit/mL.

The normal range for ADAMTS13 ranges depends upon the method to measure activity. Any art-recognized method is within the scope of the invention. In certain embodiments, the normal range in healthy individuals lies between 40-160% of a predetermined normal baseline value. See, Peyvandi et al., ADAMTS13 assays in thrombotic thrombocytopenic purpura. J Thromb Haemost. 2010 April; 8(4):631-40. In certain embodiments, the normal or baseline range in healthy individuals lies between 87-113% of a predetermined normal baseline value. See, Mancini et al., J Thromb Haemost. 2021 February; 19(2):513-521.

### Example 4: Formulation of Purified Recombinant ADAMTS13 (rA13)

Recombinant ADAMTS13 was expressed in recombinant CHO cells and purified by anion exchange chromatography. The purified rA13 had a final concentration of approximately 750 μg/ml with a specific activity of approximately 850 mU/μg. rA13 was formulated in buffer containing 150 mM NaCl, 2% sucrose, 0.05% polysorbate 80, at a pH of 7.0 with 20 mM of a buffering agent selected from (1) histidine, (2) phosphate buffer, or (3) sodium citrate. Samples were then divided evenly, and half the samples were lyophilized.

Lyophilized samples were reconstituted with sterile water to a final volume equal to that of the pre-lyophilized formulation. A single aliquot of each liquid and lyophilized formulation was then characterized by gel filtration by loading the sample onto a Superose 6 GL column (GE Healthcare). All of the formulations resulted in ADAMTS13 samples that ran as a single peak corresponding to monomeric rA13 protein by gel filtration.

In certain embodiments, histidine buffer is preferred. A suitable pH is from about 6.0 to about 8.0, preferably about 6.5 to about 8.5, and most preferably about 7.0 or 7.5. In certain embodiments, a HEPES buffer may be used (e.g. 20 mM). Optionally, suitable formulations may include EDTA and/or zinc (e.g. ZnCl₂). In certain embodiments, a liquid formulation of rA13 suitable for lyophilization, in addition to a buffer, may also contain one or more stabilizers, for example sucrose or trehalose (e.g. up to about 2%), mannitol (e.g. up to about 3%), and/or calcium (e.g. 2 mM CaCl₂). In certain embodiments, the formulation comprises about 1% sucrose and about 3% mannitol.

In certain embodiments, a liquid formulation of rA13 suitable for lyophilization comprises about 20 mM histidine, about 3% mannitol; about 0.05% polysorbate 80; about 30 or about 60 mM NaCl; about 2 or about 4 mM CaCl₂; and about 1% or about 2% sugar (e.g. sucrose or trehalose). The composition may further comprise Tween 80, e.g. 0.050%. In certain embodiments, formulations containing between about 0 mM and about 60 mM sodium chloride in the presence of about 2% sucrose produce suitable lyocakes.

In certain embodiments, a liquid formulation or reconstituted lyophilized formulation of rA13 comprises 10 mM to 50 mM histidine; 2% to 4% mannitol; 0.025% to 0.1% polysorbate 80; 0 mM to 100 mM NaCl; 2 mM to 4 mM calcium chloride; and 0.5% to 2% sucrose. In certain embodiments, the formulation further comprises between 0.5 μM and 20 μM zinc.

In certain embodiments, a liquid formulation or reconstituted lyophilized formulation of rA13 comprises 10 mM to 50 mM histidine; 2% to 4% mannitol; 0.025% to 0.1% polysorbate 80; 0 mM to 60 mM NaCl; 2 mM to 4 mM calcium chloride; and 0.5% to 2% sucrose. In certain embodiments, the formulation further comprises between 0.5 μM and 20 μM zinc.

Liquid and lyophilized formulations may be stored, for example, at room temperature (about 37-40° C.) or refrigerated (about 2-8° C.), for example 37° C. or 4° C. In certain embodiments, storage at 4° C. is preferred. In certain embodiments, the formulation is stored for up to 6, 12, 24, or 36 months. In certain embodiments, a lyophilized formulation is preferred. In certain embodiments, formulations for lyophilization having a low sodium content are preferred, e.g. up to about 100 mM NaCl, up to about 60 mM, or up to about 30 mM NaCl.

### Example 5: rADAMTS13 Formulation for Use in Treatment

A pharmaceutical composition for use in the Examples herein would be a lyophilized liquid formulation comprising an rADAMTS13 protein, with an activity of about 294 FRETS-U/ml. A suitable amino sequence for this protein would be a recombinant ADAMTS13 of human origin such as that of GenBank Accession No. NP_620594, an amino acid sequence having at least 95% sequence identity to the amino acid sequence of GenBank accession number NP_620594; a truncation of the amino acid sequence of GenBank accession number NP_620594, in which the signal peptide comprising amino acids 1-29 is removed; and a truncation of the amino acid sequence of GenBank accession number NP_620594, in which a sequence comprising amino acids 30-74 is removed. Also suitable would be a “missense” variant of any such rADAMTS13 sequence, including for example a Q⁹⁷R variant. Combinations of any of these may also be used.

Other components are Polysorbate 80 (about 0.05%), sucrose (about 1-2%), mannitol (about 3-4%), calcium (about 2-4 mM CaCl₂), sodium (about 30-60 mM NaCl), and histidine (about 20-25 mM). The pH is about 7.0-7.5, e.g. about 7.0±0.2. The composition would be stored at about 2-8° C., until ready for reconstitution use in 5 mL of sterile water for injection. Administration to a COVID-19 subject would be as a single intravenous bolus of a dose as described herein, based in IU/kg.

### Example 6: Evaluation of COVID-19 Subject for ADAMTS13 Treatment

According to this Example, a COVID-19 subject will be evaluated for ADAMTS13 treatment, based on one of more of the following criteria:

(a) whether the subject is 65 years old or older;

(b) whether the level of endogenous VWF in the subject's bloodstream is elevated, compared to a predetermined normal baseline value, according to a suitable laboratory test;

(c) whether the level of one or more cytokines in the patient's bloodstream, such as IL-8, TNF-α, and/or IL-6, is elevated, compared to a predetermined normal baseline value, according to a suitable laboratory test

(d) whether the subject has been admitted to the ICU;

(e) whether the subject is suffering from, or exhibits a sign or symptom of, one or more of a coagulopathy, blood-clotting disorder, veno-occlusive disorder, prothrombotic condition, ARDS, COPD, pneumonia, asthma, hypertension (e.g. pulmonary hypertension), thromboses (e.g. DVT), embolism (e.g. PE), myocardial infarction, and stroke (e.g. ischemic or cerebral stroke); and

(f) whether the subject has a history, predisposition, or is at risk for a coagulopathy, blood-clotting disorder, veno-occlusive disorder, prothrombotic condition, ARDS, COPD, pneumonia, asthma, pregnancy, menopause, pen-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke).

In certain embodiments, a COVID-19 subject will be evaluated for ADAMTS13 treatment based on a plurality of the above criteria.

The level of VWF would be determined according to a suitable laboratory test, as described herein.

Depending on these criteria, a COVID-19 subject may fall within one of two treatment groups, referred to herein as (1) an “earlier stage” or “maintenance” group; or (2) a “later stage” or “rescue” group.

Inclusion criteria for the “earlier stage” or “maintenance” A13 treatment group are:

(i) a history, predisposition, or risk for a coagulopathy, blood-clotting disorder, veno-occlusive disorder, prothrombotic condition, ARDS, COPD, pneumonia, asthma, pregnancy, menopause, peri-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke);

(ii) a VWF level that is not more than about 50% above a normal baseline; and

(iii) a cytokine level that is not more than about 50% above a normal baseline.

A subject meeting such criteria that is 65 years old or older is particularly indicated for treatment.

Inclusion criteria for the “later stage” or “rescue” A13 treatment group are any one or more of:

(i) diagnosis of an active coagulopathy, blood-clotting disorder, veno-occlusive disorder, prothrombotic condition, ARDS, COPD, pneumonia, asthma, pregnancy, menopause, peri-menopause, hypertension (e.g. pulmonary hypertension), thromboses (e.g. DVT), thrombotic microangiopathy (TMA), including TMA in the respiratory tract, embolism (e.g. PE), myocardial infarction, and stroke (e.g. ischemic or cerebral stroke);

(ii) a VWF level that is more than about 50% above a normal baseline; and

(iii) a cytokine level that is not more than about 50% above a normal baseline.

A subject meeting such criteria that is 65 years old or older is particularly indicated for treatment. A subject admitted to the ICU is particularly indicated for treatment. A subject exhibiting a VWF level that is at least twice a normal baseline is particularly indicated for treatment. A subject exhibiting a cytokine level that is at least twice a normal baseline is particularly indicated for treatment.

In this Example, the normal baseline for VWF levels is one of a VWF:antigen range of about 42-136% or a VWF:activity of about 42-168%. This would be determined by a laboratory test, using materials and methods known in the art, e.g. as and described herein. A normal baseline for cytokine levels and a subject's cytokine levels can be determined according to methods known in the art. This includes testing for one or more of IL-8, TNF-α, and IL-6. Normal or baseline VWF and cytokine levels may vary widely in healthy subjects. Normal A13 levels may also vary among healthy subjects. If the baseline for a particular subject is not known, the normal average, or the upper or lower end of one or more of these normal ranges would be used as a baseline. If the baseline for a particular subject is known, for example from a medical history, it may serve as a baseline for inclusion in a treatment group.

### Example 7: “Earlier Stage” or “Maintenance” Treatment for a COVID-19 Subject

A subject indicated for “earlier stage” or “maintenance” treatment would be given the rADAMTS formulation of Example 5, intravenously, in a dose having an rADAMTS13 activity of between 10 IU/kg and 80 IU/kg (by body weight), administered every other day. The dose and/or timing may be adjusted to normalize the subject to at least one of a baseline endogenous VWF levels or a baseline endogenous ADAMTS13 level. When ADAMTS13 levels are measured, the dose may be adjusted to raise ADAMTS13 levels by about 20 to 100% of average normal levels. The ADAMTS13 level would be determined by a laboratory test, using materials and methods such as those in Example 3. A normal ADAMTS13 baseline would be 100-150%; a normal average baseline would be 100% or an established reference. Dosing may differ according to sex, with women receiving a higher A13 dose, e.g. from about 5-10% higher, to align with a higher normal baseline.

A subject given “earlier stage” or “maintenance” therapy may be switched to “later stage” or “rescue” therapy if appropriate inclusion criteria are met.

### Example 8: “Later Stage” or “Rescue” Treatment for a COVID-19 Subject

A subject indicated for “later stage” or “rescue” treatment would be given the rADAMTS formulation of Example 5, intravenously, in a dose having an rADAMTS13 activity of between 40 IU/kg and 160 IU/kg (by body weight), administered once daily. The dose and/or timing may be adjusted to normalize the subject to at least one of a baseline endogenous VWF level or an absence of circulating VWF ultralarge multimers. Dosing may differ according to sex, with women receiving a higher A13 dose, e.g. from about 5-10% higher.

A subject given “later stage” or “rescue” therapy may be switched to “earlier stage” or “maintenance” therapy, if warranted by improvement from a course of “later stage” or “rescue” therapy, or if appropriate inclusion criteria are met.

### Example 9: VWF/ADAMTS13 Imbalance in COVID-19 Subjects

Thrombosis affecting the pulmonary and systemic vasculature is common during severe COVID-19 and causes adverse outcomes. Although thrombosis likely results from inflammatory activation of vascular cells, the direct mediators of thrombosis remain unconfirmed. In a cross-sectional cohort of severe COVID-19 patients, markedly increased von Willebrand factor (VWF) levels were accompanied by a partial reduction of the VWF regulatory protease ADAMTS13. This VWF/ADAMTS13 imbalance appears to be associated with persistence of circulating, highly thrombogenic, ultra-high-molecular-weight (UHMW) VWF multimers in all patients. These results implicate VWF/ADAMTS13 dysregulation in the pathogenesis of severe COVID-19 and suggest a new therapeutic target. Upon in vitro substitution of plasma from patients with severe COVID-19 with recombinant ADAMTS13 (rADAMTS13), VWF activity could be substantially reduced in a time and concentration dependent manner, visible by reduction of multimer size and depletion of UHMW VWF multimers. These data indicate that raising plasma levels of ADAMTS13 by substitution with rADAMTS13 may restore balance between VWF and ADAMTS13 in COVID-19.

In this Example, plasma from severe COVID-19 patients was incubated with rADAMTS13 under conditions which simulate cleavage of VWF by ADAMTS13 (partial VWF denaturation, e.g., with urea, to expose the cleavage site). VWF activity, VWF multimers, and ADAMTS13 activity, were compared with normal plasma controls and with plasma of thrombotic thrombocytopenic purpura (TTP) patients. Results, discussed below, indicate the small (μl) volumes of ADAMTS13 are sufficient to reduce VWF levels, particularly UHMW VWF multimers. The data show a dose response. Plasma VWF levels decline (e.g., when measured as VWF:CB) in proportion to the increasing concentration of ADAMTS13, and in a time-dependent manner.

### Methods

Patients and sample collection. Patients were eligible for the study if they were adults ≥18 years admitted to the dedicated COVID-19 intensive care units (ICUs) at either of two hospitals (n=24) or (n=12) with a seven-week period, all undergoing mechanical ventilation for severe respiratory failure. Where possible, SARS-CoV-2 infection was confirmed by PCR testing or by detection of seroconversion using specific antibody tests as described below.

Plasma samples were identified by pathology laboratory staff by selecting approximately every third new blood sample submitted for standard care coagulation tests from the COVID-19 ICUs without knowledge of the clinical characteristics of the cases. The study cohort therefore represents an otherwise unselected cross-sectional cohort of patients with a high likelihood of COVID-19 requiring ventilation and high dependency care. Clinical data were retrieved retrospectively from electronic ICU records and recorded in anonymized form onto case report forms. Ethnicity data were classified according to standardized terms utilized in the 2001 UK Census (https://www.ons.gov.uk/methodology/classificationsandstandards/measuringequality/ethnicgroupnationalidentityandreligion). The presence of Acute Respiratory Distress Syndrome was assessed using Berlin criteria. See, Force et al., JAMA 307 (23) (2012) 2526-2533. Evidence of additional bacterial sepsis was detected from inspection of contemporaneous clinical case records and pathology records such as blood culture or bacterial sepsis biomarker results. Analyses were performed on de-identified spare plasma that remained after performing standard care diagnostic tests. Sample analysis and the linkage to clinical data were registered as service evaluation projects and were exempted from research ethics committee approval and patient consent by the host organizations in accordance with UK NHS Health Research Authority guidance (N.H.R. Authority. https://www.hra.nhs.uk/; Accessed 7 Sep. 2020). The data collection and laboratory analyses were registered as service evaluation exercises at the host institutions, which confirmed that research ethics committee approval and patient consent was waived.

SARS-CoV-2 detection. SARS-CoV-2 RNA was detected from nasopharyngeal swabs by PCR using the Aptima SARS-CoV-2 Assay on a Hologic Panther System (Hologic, Marlborough, Mass., USA). SARS-CoV-2 seroconversion was detected using a novel serological assay reported recently (Goshua et al., supra). The test system consists of two independent ELISA antibody titres of >5 U/mL in both are required to indicate seroconversion of a patient. Specific SARS-CoV-2 patient antibodies are bound in the TECHNOZYM anti SARS CoV 2 RBD IgG ELISA (Technoclone, Vienna, Austria) using immobilized recombinant SARS-CoV-2 RBD and in the TECHNOZYM anti SARS CoV 2 NP IgG ELISA (Technoclone) using immobilized recombinant nuclear SARS-CoV-2 protein. In both ELISAs, the captured patient antibodies are detected using an anti-human IgG HRP labelled polyclonal antibody and quantified using SARS-CoV-2 positive serum standardized against a specific monoclonal antibody for RBD (CR3022).

Coagulation protein analysis. Blood samples collected as part of routine clinical care into 3.2% Trisodium citrate were centrifuged at 2400 g for 10 minutes to yield plasma aliquots. The samples were stored at −80° C. or in temperature monitored dry ice until batched analysis. The prothrombin time, activated partial thromboplastin time, Clauss fibrinogen and D dimer were measured using a CS-2500 series analyser (Sysmex Corp. Kobe, Japan) using Siemens Healthcare reagents (Marburg, Germany) according to the manufacturers' instructions. The Factor VIII activity was determined using a one-stage activity assay with Actin FS activator and FVIII deficiency plasma from Siemens Healthcare.

Platelet counts. Platelet counts were measured in EDTA blood samples using a XN series analysers (Sysmex). ABO blood groups were determined from EDTA-anticoagulated blood samples with a IH-1000 instrument (Bio-Rad Laboratories, Watford, United Kingdom). Core hematological and coagulation tests were performed using Sysmex XN and CS-2500 analyses (Sysmex corp. Kobe, Japan).

Von Willebrand factor assays. VWF:Ag was determined from eight different dilutions of patient plasma using the Asserachrom VWF:Ag test kit (Diagnostica Stago, Asnieres sur Seine, France) with human normal plasma as the assay reference standard. VWF ristocetin cofactor activity (VWF:RCo) was measured using the BCS coagulation system analyzer (Behring Coagulation System, BCS, Siemens, Germany) and a reference plasma, calibrated against the WHO standard. VWF collagen binding (VWF:CBA) was determined using the TECHNOZYM VWF:CBA test kit (Technoclone, Vienna, Austria) or using the Zymutest test kit (HYPHEN BioMed SAS, Neuville sur Oise, France) using assay calibrators traceable to the WHO standard.

ADAMTS13 assays. Three complementary assays were used to determine activity of ADAMTS13 all obtained from Technoclone (Vienna, Austria), TECHNOZYM ADAMTS13 Activity ELISA, TECHNOFLOUR ADAMTS13 Activity, and TECHNOZYM ADAMTS13 Antigen (See, Example 10). The plasma ADAMTS13 activity was determined using both ELISA and FRETS methods. The ADAMTS13 protein level was determined by ELISA. The VWF and ADAMTS13 assays used human plasma references wherever possible calibrated against World Health Organisation reference preparations. The COVID-19 patient samples were analyzed alongside a control plasma sample from a 48-year-old male with acute phase autoimmune TTP (known ADAMTS13 level <0.01 IU/mL) and a high spin normal pooled plasma sample (normal ADAMTS13 of 0.9-1.0 IU/mL; Precision BioLogic, Dartmouth, NS, Canada).

VWF multimers. Analysis of VWF multimeric composition was performed using both semi-automated electrophoresis gels and home cast low-resolution 1% and high-resolution 2.5% SDS agarose gels. These complementary electrophoretic methods were selected to reduce experimental bias common to electrophoretic analyses. The latter method has been optimized to separate even the largest molecular sized multimers ranging at or above 20 MDa and enabled quantitative determination of multimers higher than those present in normal human reference plasma.

Multimer composition. VWF multimeric composition was determined in home cast low resolution SDS-agarose electrophoresis gels followed by immunodetection with a commercially available polyclonal anti-VWF antibody as described previously (South, supra). In contrast to analyzer-based methods where samples are kept in containment, electrophoresis is an open system. To reduce infectivity samples were thus heat-inactivated before analysis for 1 hour at 60° C. in Tris/EDTA/SDS/agarose. Before pipetting into the sample slots, patient plasmas were diluted according to their antigen level so that on each lane a sample with the same concentration of 1 IU VWF:Ag/mL was applied. This enables direct visual comparison and quantitative densitometric scanning of sample lanes because irregular staining in the gel could be avoided. Low and high separation conditions used 1% and 2.5% Seakem agarose (Lonza, Basel, Switzerland). Multimer bands were detected by in-gel 2-step immunostaining with an anti-VWF antibody (DAKO, Glostrup; Denmark) followed by a goat-anti-rabbit ALP conjugate (Jackson, West Grove, Pa., USA). Bands were visualized with AP conjugate substrate kit (Bio Rad, Vienna, Austria).

Multimer visualization. Multimer patterns were evaluated visually and quantitatively by densitometry (GS-900, BioRad, CA, USA). For the largest multimers, the migration distance from sample application was measured and the relative mobility (Rf) was calculated. Rf of the VWF dimer was assigned a migration value of 1.0. Then the Rf of the highest stained portion in each lane resembling the largest multimers was calculated and subtracted (1-Rf). The 1-Rf value of normal human plasma applied as control on each gel was used as reference and was defined as containing 100% of multimers. The maximum size of VWF multimers measured as 1-Rf detected in each sample lane was quantified relative to the normal human plasma lane on each gel. This method enables quantitative comparison of samples separated on different agarose gels in different electrophoresis runs. In order to enable comparison between different electrophoresis gel runs this value was reported as a percentage of the 1-Rf value for a normal plasma sample separated on the same gel (termed hereafter the UHMW multimer quantitation parameter). An increased percentage value indicates UHMW VWF multimers in the test sample

The findings from the home cast gel electrophoresis were validated using a semi-automated electrophoresis gel method using the HYDRAGEL von WILLEBRAND MULTIMERS kit and a HYDRASYS 2 SCAN instrument (Sebia, Lisses, France). This method separates VWF multimers in the high to ultra-large range less well than the home cast 1% agarose gels but shows better reproducibility between gels.

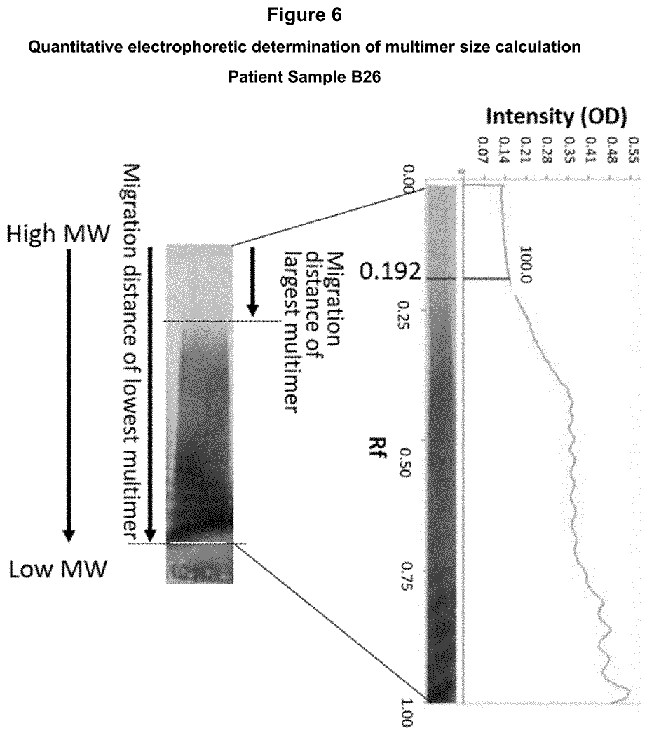

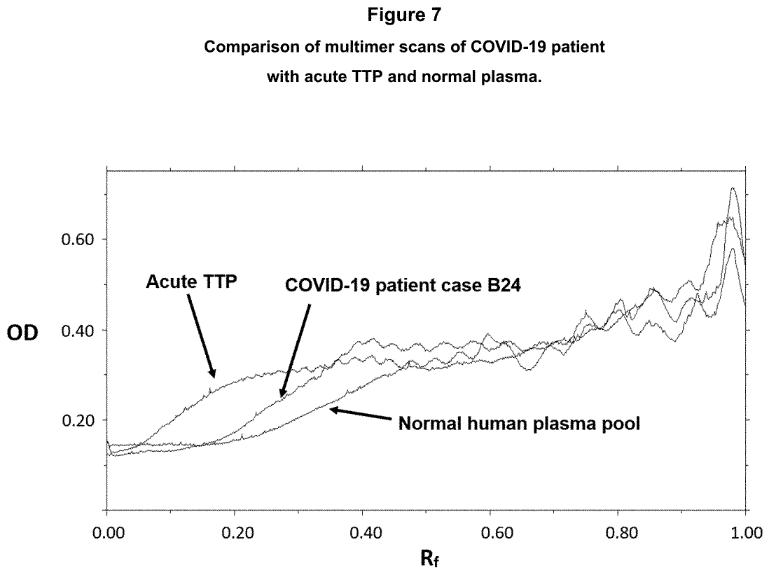

Densitometry. An illustration of this quantitative determination of multimers on an agarose gel, where a sample of one of the investigated patients had been applied, is shown in FIG. 6 (patient B4). Patient plasma adjusted to a concentration of 1 IU/mL VWF:Ag was separated on a 1% agarose gel. After immunostaining densitometry, relative migration of largest multimer (Rf) was calculated at 0.192 defined according to the absorbance in the densitogram (see methods S8). Rf of lowest multimer (=dimer) was defined as 1.0. 1−Rf was calculated at 0.808 (=1.000-0.192). 1-Rf for normal human plasma references separated on the same gel was calculated at 0.729 (not shown in graph). Relative multimer size of sample B24 resulted at 111% (0.808/0.729*100=111%). FIG. 7 shows another example of densitometric scans of a COVID-19 patient, in direct comparison to the acute TTP control, and to the normal human plasma pool. In this overlay of densitometric scans of the sample of COVID-19 patient B24, the acute TTP sample, and the normal human plasma pool, the COVID-19 patient had larger multimers than the normal control but less than a patient with acute TTP.

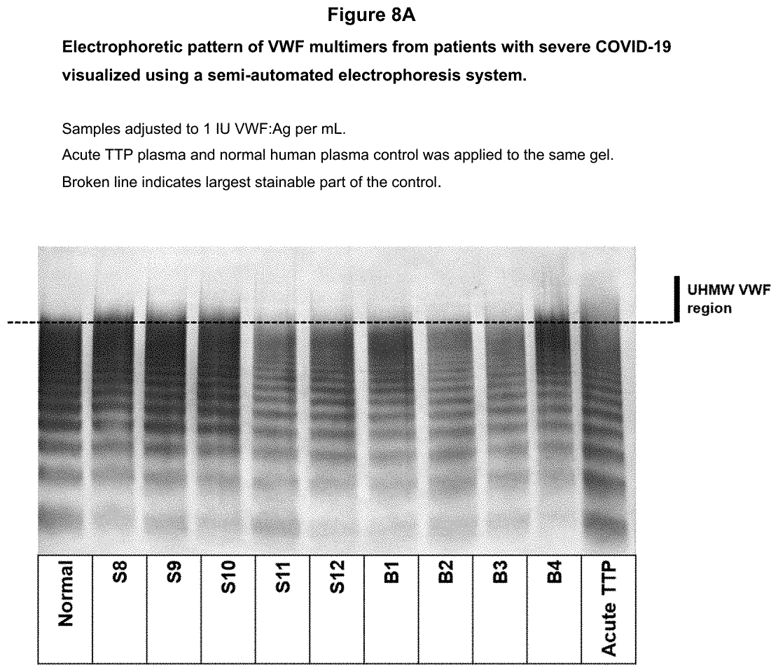

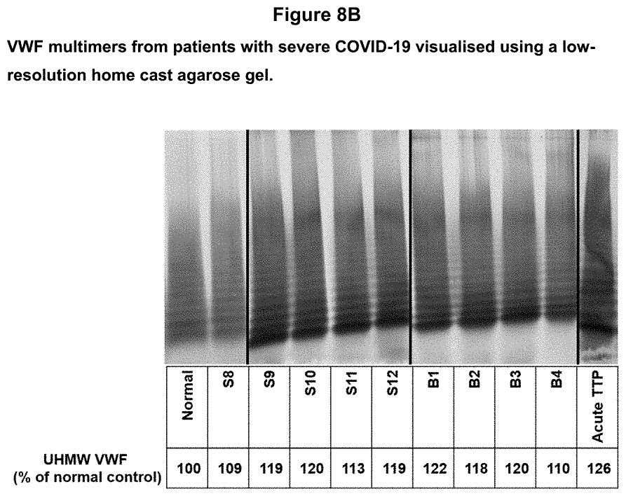

Visual confirmation. The HYDRAGEL von WILLEBRAND MULTIMERS kit (Sebia, Lisses, France) was used to confirm the results obtained by the home cast agarose gels. The semi-automated electrophoresis system followed by immunofixation, using the HYDRASYS 2 instrument allows visual assessment of the overall size distribution of VWF multimers. Analysis was performed according to the manufacturer's instructions. This alternative agarose gel electrophoresis method does not separate VWF multimers in the high to ultralarge range with the same resolution as the home cast 1% agarose gels, but shows better reproducibility from gel to gel, thus validating the results obtained using the home cast gels. FIG. 8A shows an example where plasma of COVID-19 patients, each adjusted to 1 IU VWF:Ag per mL, was separated and stained for multimers parallel to acute TTP and normal controls. Plasma samples of 9 patients with COVID-19 were separated by semi-automated electrophoresis. Each sample was adjusted to 1 IU VWF:Ag per mL, separated, and stained for multimers in parallel. Acute TTP plasma and a normal control samples were applied to the same gel. The broken line in FIG. 8A indicates the largest stainable part of the normal human plasma control.

rADAMTS13. Purified recombinant human ADAMTS13 (rADAMTS13, Takeda, Vienna, Austria) from a Chinese hamster ovary cell line has previously been described. See, Plaimauer et al., Blood. 2002; 100(10):3626-3632; Rottensteiner et al., J Thromb Haemost. 2019; 17(12):2099-2109; Scully et al., Blood. 2017; 130(19):2055-2063. The rADAMTS13 product used in this study was the same material used in clinical trials for patients with hereditary thrombotic thrombocytopenic purpura (ClinicalTrials.gov Identifier: NCT02216084) and patients with sickle cell disease (ClinicalTrials.gov Identifier: NCT03997760).

VWF cleavage studies. Degradation of VWF with rADAMTS13 was performed under conditions where VWF is partially denatured to make the cleavage motif accessible for the protease. The rADAMTS13 (Takeda, Vienna, Austria) was pre-diluted to 10 and 100 IU/mL FRETS-VWF73 activity (final concentrations between 0.5 and 10 IU/mL) and activated with BaCl₂ in the presence of 5 mM Tris and 1.5 M urea, pH 8.0, at 37° C. for 30 minutes. Activated rADAMTS13 was mixed 1+9 with patient plasma samples and further incubated at 37° C. for 2 and 5 hours before proteolysis. Number of time points and concentrations of rADAMTS13 was limited by the availability of plasma volume from the patients. Reaction was stopped by the addition of Na₂SO₄ (8.25 mM final concentration). To measure the effect of endogenous ADAMTS13 of the plasma, patient samples were diluted with buffer instead of rADAMTS13, and the same procedure was followed as that with rADAMTS13. Samples were centrifuged at 2500×g for 5 minutes and the VWF in the supernatants was analyzed using the VWF:CB assay and by quantitative densitometry of semi-automated electrophoresis gels performed with the Sebia Phoresis rel. 9.2.0 software to calculate relative increase of VWF dimer levels relative to the total quantity of VWF determined from the areas under the densitometry curves. UHMW VWF multimers were quantified from the home case gels as described above.

Statistical analysis. Descriptive statistics and Pearson correlation analysis were performed to determine correlations (r) between ADAMTS13 and VWF levels measured with the different test methods. In order to investigate the extent of degradation of VWF by rADAMTS13 in the ex vivo VWF cleavage experiments, paired t-tests were used to compare VWF:CB assay data (10 samples) and relative VWF dimer content from semi-automated electrophoresis gels (8 samples) at baseline and after 5 h of incubation with 1 IU/mL rADAMTS13. All the statistical analyses were performed using Minitab version 18.1.

### Results

All 36 patients enrolled into the investigation displayed a rapidly progressive febrile illness with cough, shortness of breath and pulmonary infiltrate progressing to severe respiratory failure that are characteristics of severe COVID-19. Additionally, 34 patients had a positive SARS-CoV-2 PCR test of which 30 had developed specific SARS-CoV-2 antibodies. One patient had a negative PCR test but displayed high levels of SARS-CoV-2 antibodies consistent with infection. A positive detection test was not obtained from a single patient who died from rapidly progressive COVID-19 before PCR confirmation could be performed. The patient cohort spent a median of 16 days on the ICU before discharge to a low dependency ward or death. Blood samples for laboratory analysis were collected a median of 10 days (range 0-57 days) after ICU admission. All except two patients were receiving either prophylactic or therapeutic doses of parenteral anticoagulants at the time of blood sampling. It is noteworthy that all the patients displayed greatly elevated plasma fibrinogen, D-dimer levels and Factor VIII activity consistent with severe activation of the hemostasis pathway. No patients had the laboratory features of disseminated intravascular coagulation according to ISTH criteria. See, Suzuki et al., J Thromb Haemost 2018 July; 16(7):1442-1444. The clinical and core laboratory characteristics of the study cohort are reported in Table 2. The clinical data are number (%). The laboratory data are median (range) reported alongside reference intervals derived from healthy controls. ICU=intensive care unit; LMWH=low molecular weight heparin; UFH=unfractionated heparin; PT=prothrombin time; APTT=activated partial thromboplastin time. ^{a} Classified according to Berlin ARDS criteria. See, Force et al., J Thromb Haemost. 2018 July; 16(7):1442-1444. ^{b} UK Blood donor registry expected frequencies.

TABLE 2
Clinical and core laboratory characteristics of the study group

| | | |
|---|---|---|
| Age (years) | 61 | (23-76) |
| Male sex | 28 | (78%) |
| Ethnicity | | |
| White British | 19 | (53%) |
| Black or Black British | 10 | (28%) |
| Asian- Pakistani or British Pakistani | 3 | (8%) |
| Other | 4 | (11%) |
| Co-morbidities | | |
| Diabetes | 15 | (42%) |
| Hypertension | 14 | (39%) |
| Obesity | 9 | (25%) |
| Cardiovascular disease | 9 | (25%) |
| Chronic kidney disease | 8 | (22%) |
| Immunocompromised | 3 | (8%) |
| Active malignancy | 3 | (8%) |
| Thrombosis | | |
| Peripheral vein thrombosis | 8 | (22%) |
| Pulmonary embolus or thrombosis | 7 | (19%) |
| Stroke | 3 | (8%) |
| No confirmed thrombosis | 19 | (53%) |
| Anticoagulation at time of blood sample | | |
| Prophylactic LMWH or UFH | 22 | (61%) |
| Treatment LMWH, UFH or | 12 | (33%) |
| Argatroban | 2 | (6%) |
| No anticoagulant | | |
| Outcome | | |
| Death | 19 | (53%) |
| Discharged from ICU | 17 | (47%) |

| | | | |
|---|---|---|---|
| ABO blood group O | 13 | (36%) | 48%^{a} |
| group A, B or AB | 23 | (64%) | 52%^{b} |

| | |
|---|---|
| Patient | Reference |
| value | range |

| | | | |
|---|---|---|---|
| PT (s) | 11.8 | (10.9-37.7) | 9.5-12.0 |
| APTT (s) | 28.8 | (20.5-71.8) | 23.0-32.0 |
| Clauss fibrinogen (g/dL) | 6.4 | (3.2-9.0) | 1.5-4.5 |
| Platelet count (x10⁹/L) | 231 | (11-621) | 150-400 |
| D-dimer (ng/mL) | 3128 | (235-13280) | <500 |
| Factor VIII activity- clotting (IU/mL) | 4.71 | (2.14-9.98) | 0.50-2.00 |

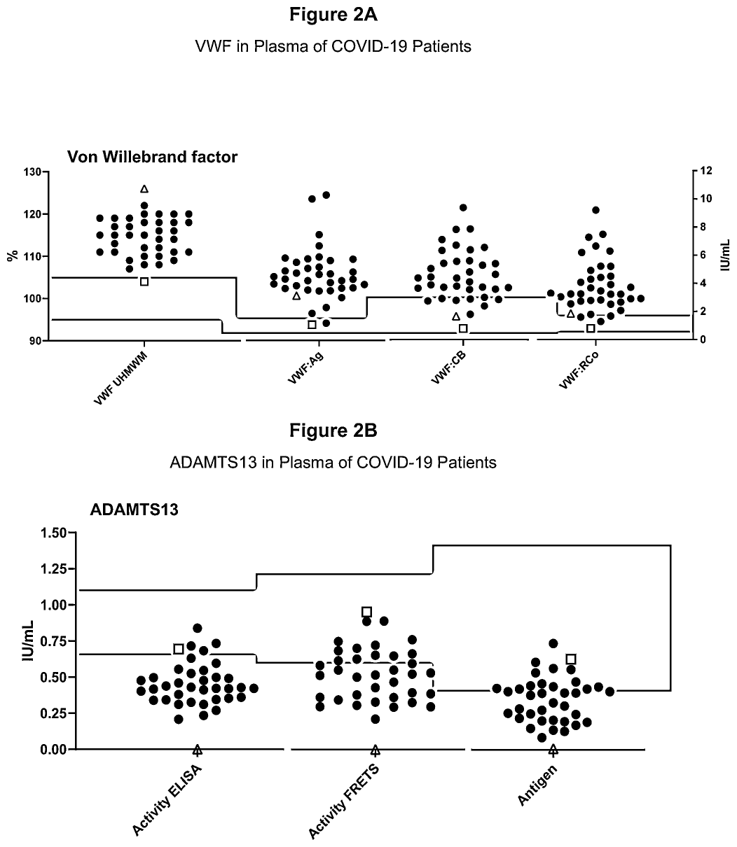

All of the COVID-19 patients had plasma VWF protein levels (VWF: Ag) above the upper limit of the laboratory reference interval derived from healthy volunteer plasma samples. In one third of cases, VWF levels were more than five-fold higher than average normal levels and in two cases were more than ten-fold higher (Table 3 and FIG. 2A). There was a corresponding dramatic increase in plasma VWF activity determined using both the VWF:RCo and VWF:CB assays.

By contrast, levels of both ADAMTS13 protein (ADAMTS antigen) and activity determined both by the ELISA and FRETS assays were, in most patients, reduced to between 30 to 70 percent of average normal levels (Table 3 and FIG. 2B). No COVID-19 patients displayed ADAMTS13 activity levels less than 20 percent of average normal levels, distinguishing these from the autoimmune TTP control in which ADAMTS13 activity was undetectable by any of the assays.

Table 3 shows VWF and ADAMTS13 laboratory parameters of 36 COVID-19 patients. Summary statistics presented as median and range alongside reference intervals obtained from analysis of healthy volunteer samples. The UHMW VWF multimer quantitation parameter is the proportion of the migration distance of the VWF dimers that was occupied by all VWF multimers in each sample lane expressed as a percentage value of that in the healthy control plasma lane from the same gel. VWF—von Willebrand factor; UHMW—ultra high molecular weight; ADAMTS13—a disintegrin and metalloprotease with a thrombospondin type 1 motif member 13; n.a.—not applicable. * normal human plasma samples are devoid of detectable UHMW VWF multimers due to processing; the provided range accounts for normal variation. ** mean of the Reference range.

TABLE 3
Characterization of the von Willebrand factor/ADAMTS13 axis

| Laboratory | Patient value (n = 36) | | Reference | Reference |
|---|---|---|---|---|
| parameter | Median | Range | range | value** |
| VWF antigen | 4.45 | 1.22-10.32 | 0.50-1.60 | 1.05 |
| (IU/mL) | | | | |
| VWF ristocetin co- | 3.27 | 1.28-9.20 | 0.58-1.72 | 1.15 |
| factor activity | | | | |
| (IU/mL) | | | | |
| VWF collagen | 4.38 | 1.80-9.38 | 0.40-2.50 | 1.45 |
| binding activity | | | | |
| (IU/ml) | | | | |
| ADAMTS13 | 0.42 | 0.21-0.84 | 0.66-1.10 | 0.88 |
| activity ELISA | | | | |
| (IU/mL) | | | | |
| ADAMTS13 | 0.52 | 0.21-0.89 | 0.60-1.21 | 0.91 |
| activity FRETS | | | | |
| (IU/mL) | | | | |
| ADAMTS13 | 0.38 | 0.08-0.73 | 0.41-1.41 | 0.91 |
| antigen (IU/ml) | | | | |
| UHMW VWF | 36 | n.a. | n.a. | n.a. |
| multimers present | (100%) | | | |
| by visual | patients | | | |
| examination and | and TTP | | | |
| densitometry | control | | | |
| UHMW VWF | 115 | 107-122 | 95-100* | 100 |
| multimer | | | | |
| quantitation (%) | | | | |

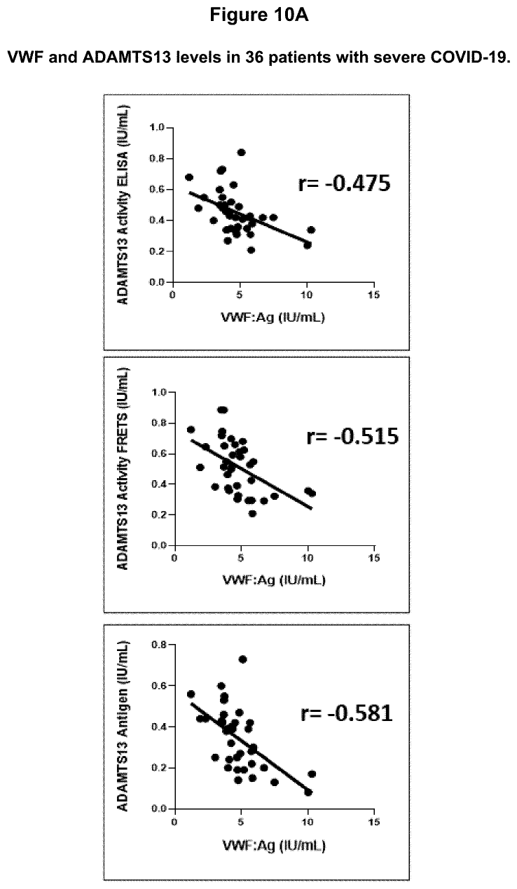

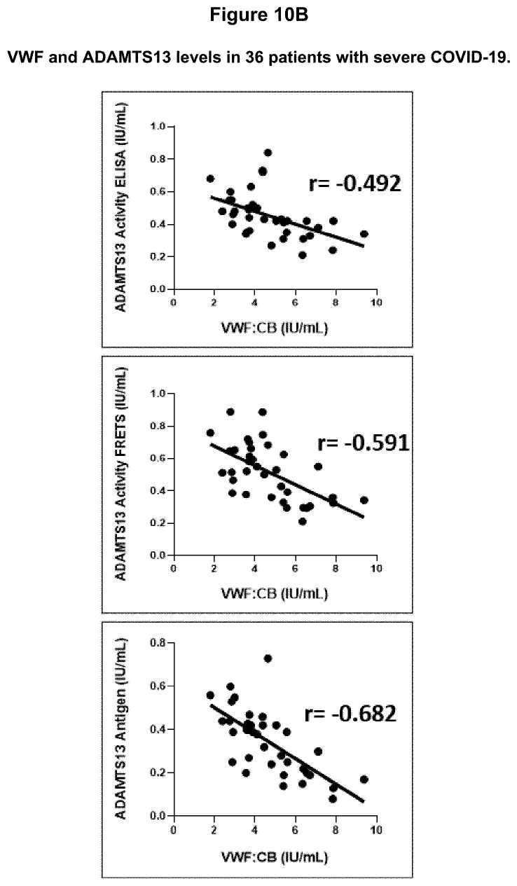

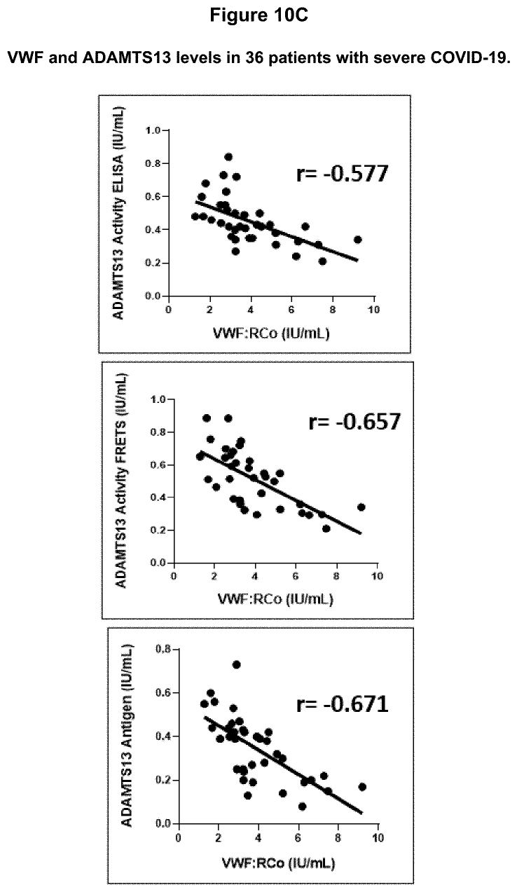

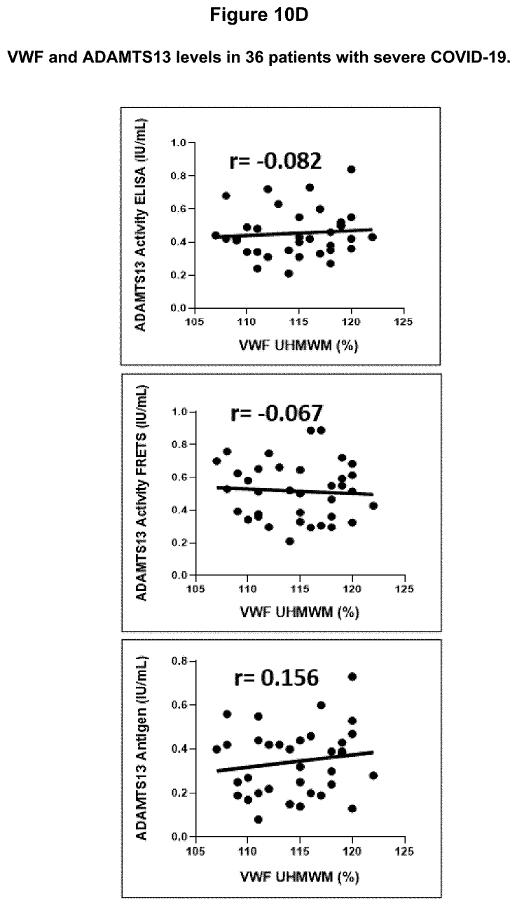

VWF levels determined using activity antigen tests showed a negative correlation with ADAMTS13 levels (Table 4 and FIGS. 10A-10D). Table 4 shows the correlation estimates as r-values and associated p-values. Correlations were classified as strong (r: 0.7 to 1.0), moderate (r: 0.5 to 0.7), weak (r: 0.3 to 0.5) or no correlation (r: 0 to 0.3).

TABLE 4
Correlation analyses of ADAMTS13 and VWF levels
of the 36 patients with severe COVID-19

| Parameter | Pearson r | p-value | Correlation |
|---|---|---|---|
| ADAMTS13 activity ELISA/VWF:Ag | −0.475 | 0.003 | moderate |
| ADAMTS13 activity FRETS/VWF:Ag | −0.515 | 0.001 | moderate |
| ADAMTS13 antigen/VWF:Ag | −0.581 | <0.001 | moderate |
| ADAMTS13 activity ELISA/VWF:CB | −0.492 | 0.002 | moderate |
| ADAMTS13 activity FRETS/VWF:CB | −0.591 | <0.001 | moderate |
| ADAMTS13 antigen/VWF:CB | −0.682 | <0.001 | strong |
| ADAMTS13 activity ELISA/VWF:RCo | −0.577 | <0.001 | moderate |
| ADAMTS13 activity FRETS/VWF:RCo | −0.657 | <0.001 | strong |
| ADAMTS13 antigen/VWF:RCo | −0.671 | <0.001 | strong |
| ADAMTS13 activity ELISA/% VWF | −0.082 | 0.634 | no correlation |
| UHMWM | | | |
| ADAMTS13 activity FRETS/% VWF | −0.067 | 0.699 | no correlation |
| UHMWM | | | |
| ADAMTS13 antigen/% VWF UHMWM | 0.156 | 0.362 | no correlation |

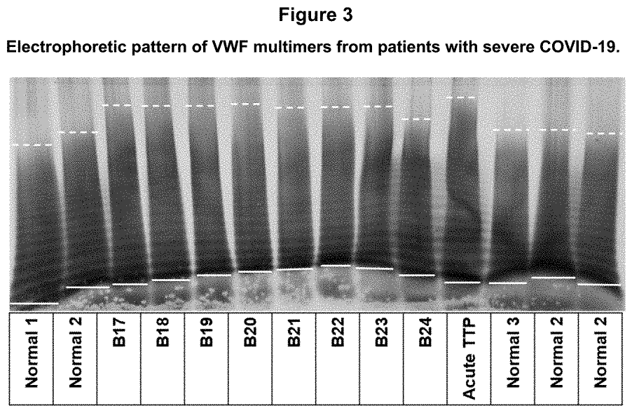

Visual inspection of the immunostained low-resolution agarose gels showed that UHMW VWF multimers were present in all 36 of the COVID-19 patients and in the control patient with autoimmune TTP, but were absent in the control pooled human reference plasma (FIG. 3). This was quantified by measuring the distance of the fastest migrating VWF band (corresponding to VWF dimers) and the slowest migrating band (the highest multimer size present) on the electrophoresis gels (FIG. 6 and VFW methods disclosed herein). In the COVID-19 samples, the distances between the highest and lowest bands ranged between 107 and 122 percent of that in control plasma because of the additional UHMWM bands (FIG. 2A). The highest elevation in the COVID-19 samples approached that of the control with autoimmune TTP (126 percent). The methodology for quantitation of UHMW multimers from illustrative case B26 is also shown in FIG. 6. The presence of UHMW VWF multimers did not correlate with ADAMTS13 levels (Table 4 and FIGS. 10A-10D), reflecting that although quantitation of UHMW VWF forms using gel electrophoresis is robust within individual samples, comparison of values between different samples is limited by intrinsic variation in staining intensity between gel lanes.

The finding of abnormal UHMW VWF multimers was confirmed by quantitative densitometric analysis of the home-cast gels and by visual inspection of the independent semi-automated electrophoresis assay (FIGS. 8A and 8B). The semi-automated system improves resolution of lower molecular weight VWF multimers and was used to quantify VWF dimers in the subsequent rADAMTS13 incubation experiments. This high-resolution system is less suitable to separate the UHMW VWF multimers which have a molecular weight of approximately 20 MDa. However, although unsuitable for quantitation of UHMW VWF multimers, the semi-automated gels were sufficient to enable visual confirmation of UHMW multimers in all of the COVID-19 patient samples. Illustrative examples of semi-automated electrophoresis for the COVID-19 samples and quantified UHMW multimers determined from the corresponding home-cast gels are shown in FIGS. 8A and 8B, respectively.

To investigate the effect of restoring the low ADAMTS13 levels on VWF in the patient samples, ten plasma samples from different COVID-19 patients with high and very high VWF levels were incubated with different concentrations of rADAMTS13 and were monitored for VWF activity and multimer structure were monitored over time. FIG. 4A shows the time-dependent reduction of VWF activity in a patient with 4.36 IU/mL VWF:Ag levels, 0.59 IU/mL ADAMTS13 activity, and VWF/ADAMTS13 ratio 7.4. Some degradation was seen without supplementation of exogenous rADAMTS13 due to presence of endogenous ADAMTS13 activity. Specifically, there was a small reduction in VWF activity at 2 and 5 h consistent with the presence of endogenous ADAMTS13.

Increasing ADAMTS13 levels to average normal (approx. 1 IU/mL=100%) resulted in higher reduction of VWF activity which could be further reduced by supplementing to about 150%. After supplementation with 0.5 or 1 IU/mL rADAMTS13, predicted to restore plasma ADAMTS13 to approximately 100% (1 IU/mL) and 150% (1.5 IU/mL) of average normal levels, respectively, there was an accelerated reduction in VWF activity in a dose and time dependent manner (FIG. 4A).

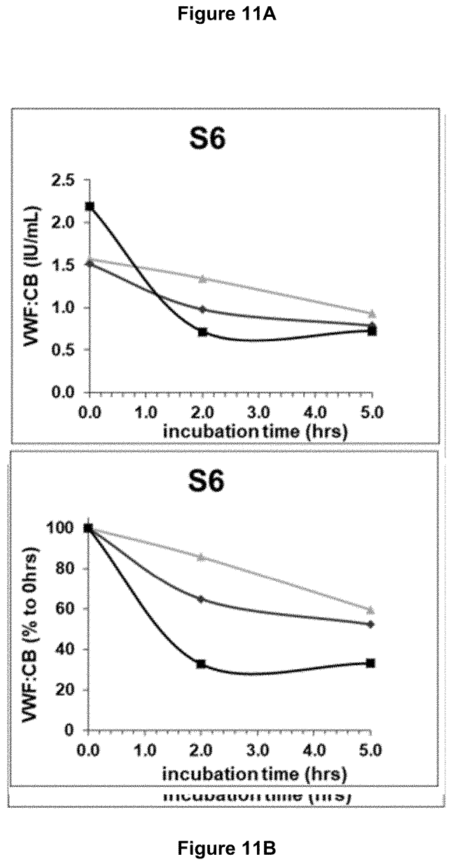

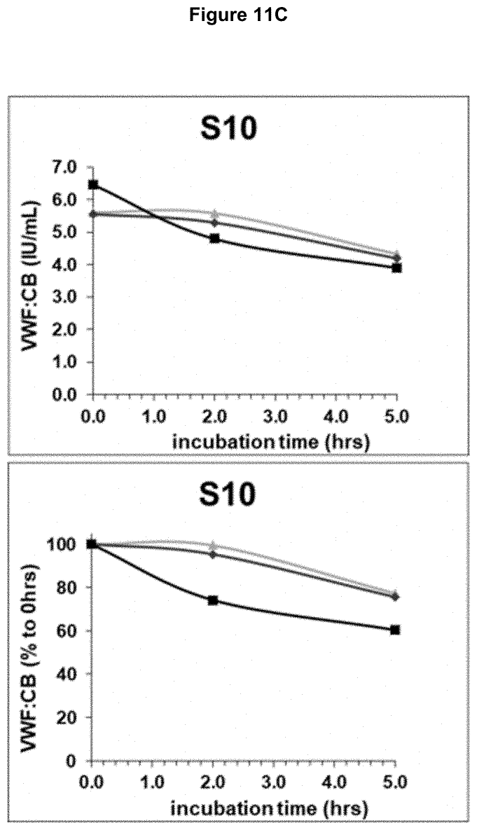

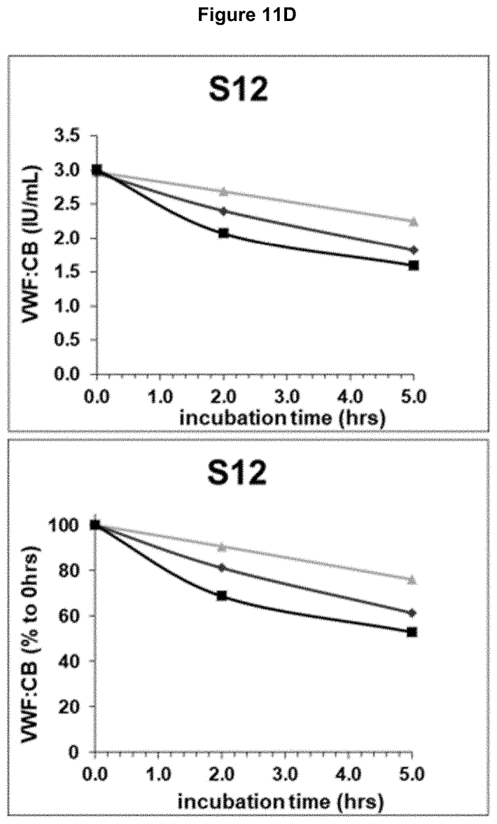

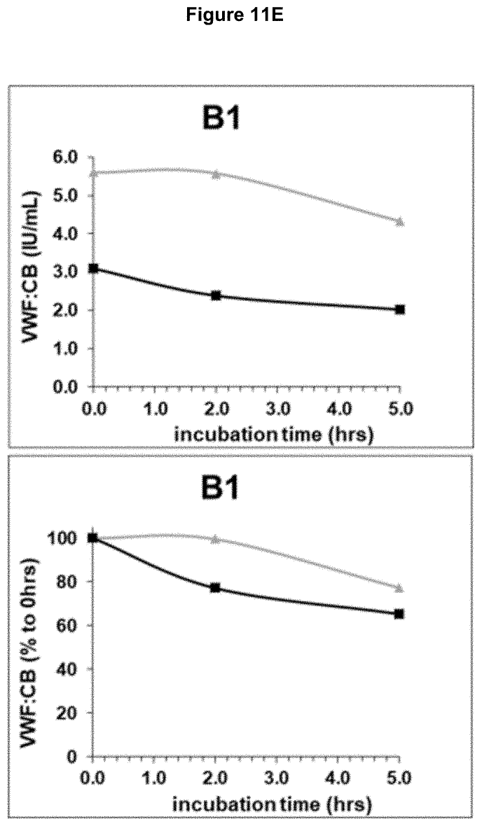

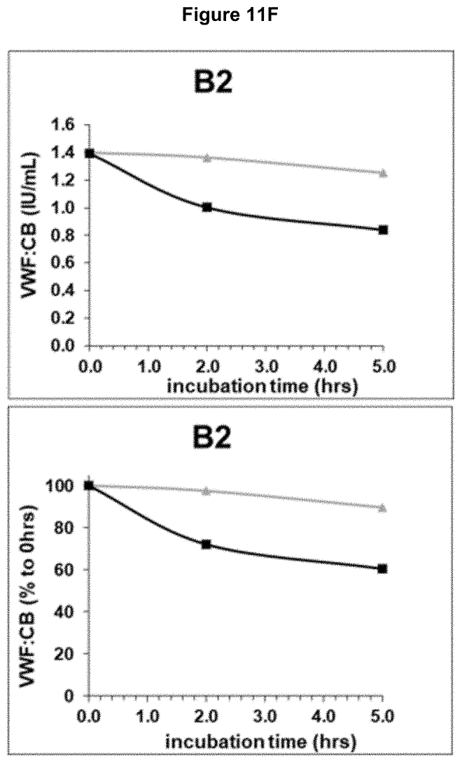

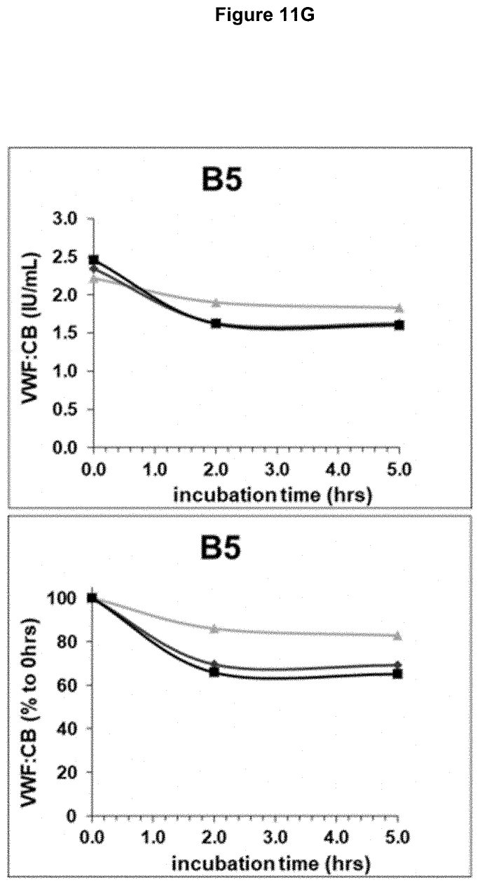

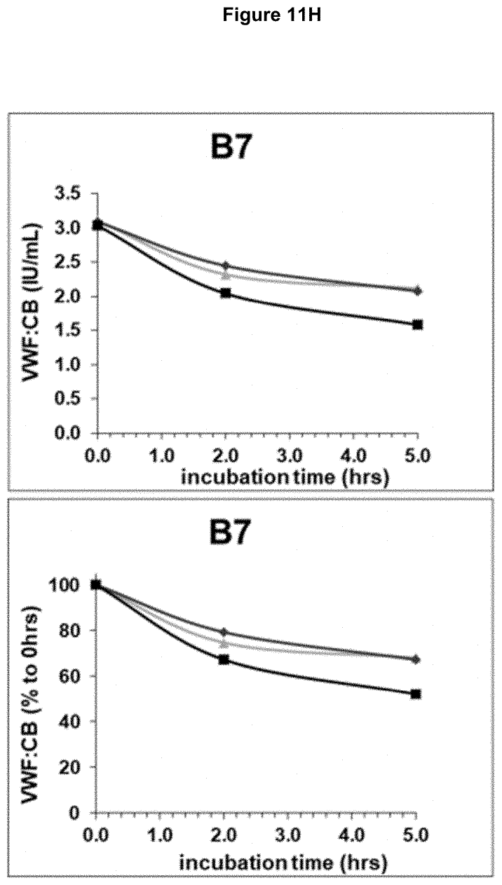

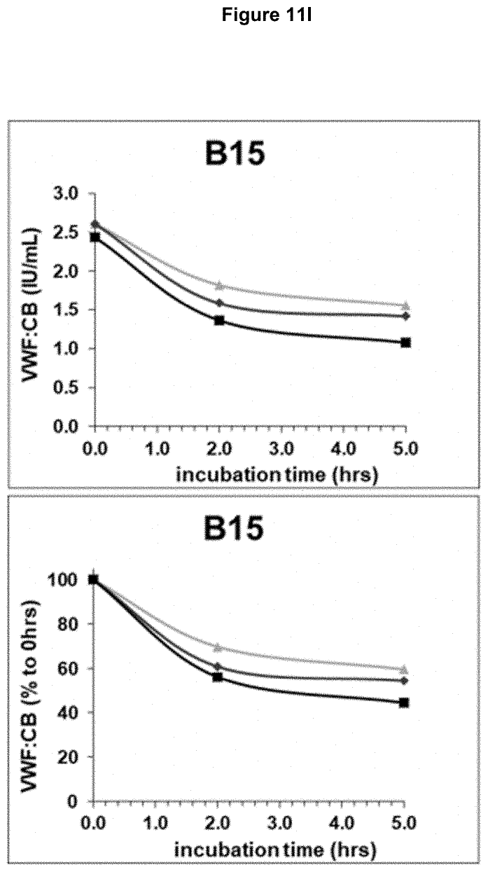

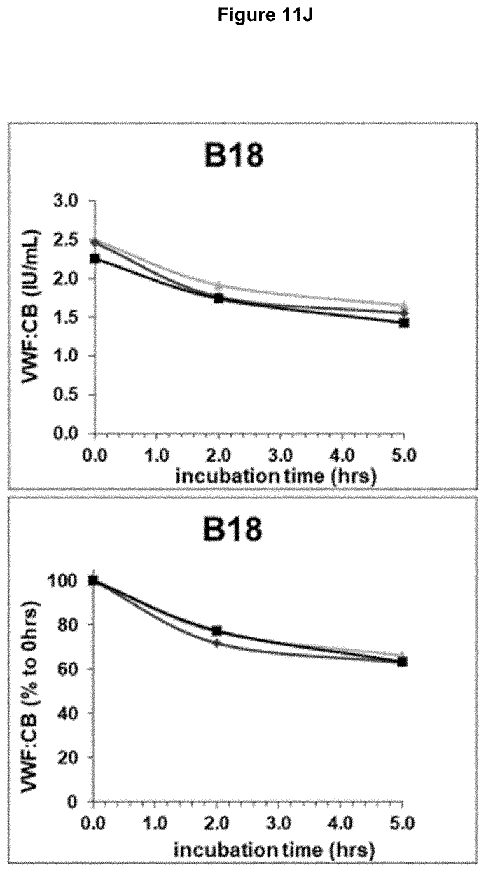

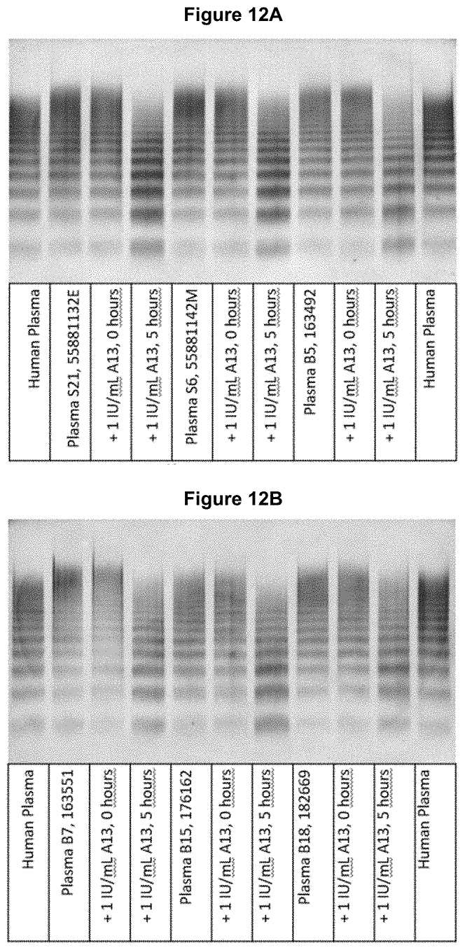

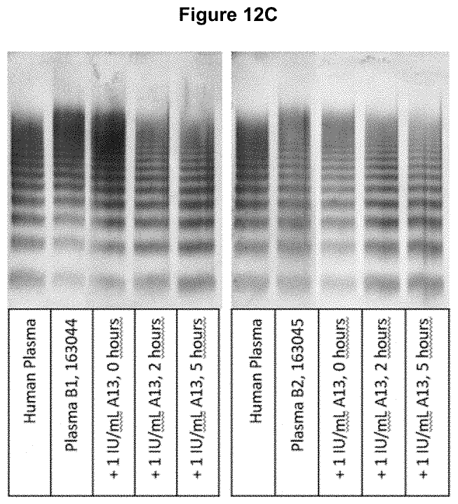

After incubation with 1 IU/mL rADAMTS13 for 5 h, the mean value of VWF:CB in the samples from all ten patients that were tested decreased from 3.01 (SD: 1.37; SEM: 0.434) to 1.64 (SD: 0.89; SEM 0.28) IU/mL (p<0.001; t-value 7.18; FIGS. 4B and 4C and Table 5). Individual graphs of these data were generated for each of the ten patients (FIGS. 11A-11J), and quantitative multimer analyses were also performed for a subset of eight patients (FIGS. 12A-12B).

The observed decrease in VWF activity after incubation with 1 IU/mL rADAMTS13 was accompanied by a statistically significant increase (P<0.001; t-value −7.04) in VWF dimers measured by densitometry, as exemplified in Table 5. n.d. indicates not done; n.a. indicates not applicable.

TABLE 5
Results of VWF cleavage studies of ten plasma
samples of COVID-19 patients with rADAMTS13

| Patient | | | Decrease (%) | Dimer relative to total | | Increase (%) |
|---|---|---|---|---|---|---|
| plasma | VWF:CB | | of VWF:CB 0 | multimer content | | of dimer 0 |
| no. | (IU/mL) | | vs. 5 hrs | (AUC %) | | vs. 5 hrs |
| — | 0 hrs | 5 hrs | — | 0 hrs | 5 hrs | −5.76 |
| S21 | 3.70 | 1.63 | 44 | 4.9 | 8.5 | 173 |
| S6 | 2.19 | 0.73 | 33 | 5.4 | 7.0 | 130 |
| S10 | 6.48 | 3.91 | 60 | n.d. | n.d. | n.a. |
| S12 | 3.01 | 1.60 | 53 | n.d. | n.d. | n.a. |
| B1 | 3.09 | 2.02 | 65 | 3.6 | 9.1 | 253 |
| B2 | 1.39 | 0.84 | 60 | 5.4 | 11.3 | 209 |
| B5 | 2.46 | 1.61 | 65 | 3.7 | 11.0 | 297 |
| B7 | 3.03 | 1.58 | 52 | 5.3 | 10.1 | 191 |
| B15 | 2.44 | 1.08 | 44 | 5.1 | 12.3 | 241 |
| B18 | 2.25 | 1.42 | 63 | 3.6 | 7.0 | 194 |

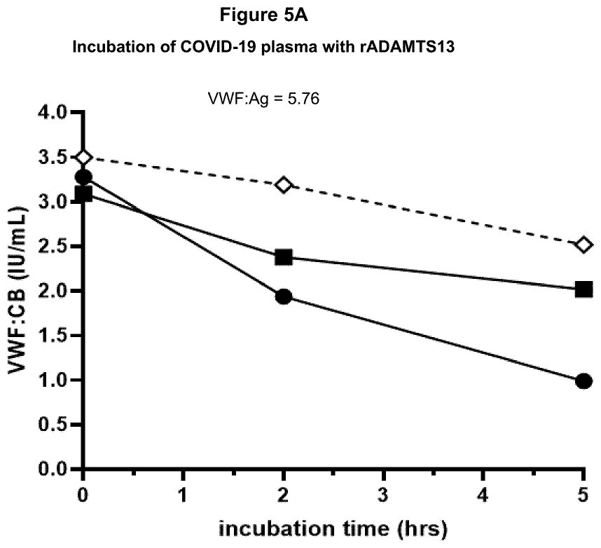

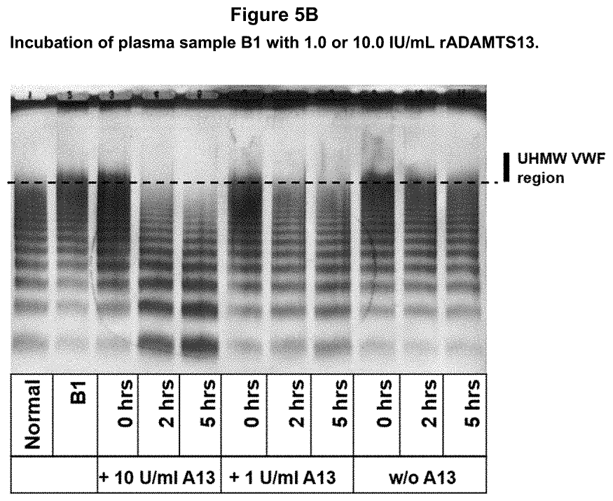

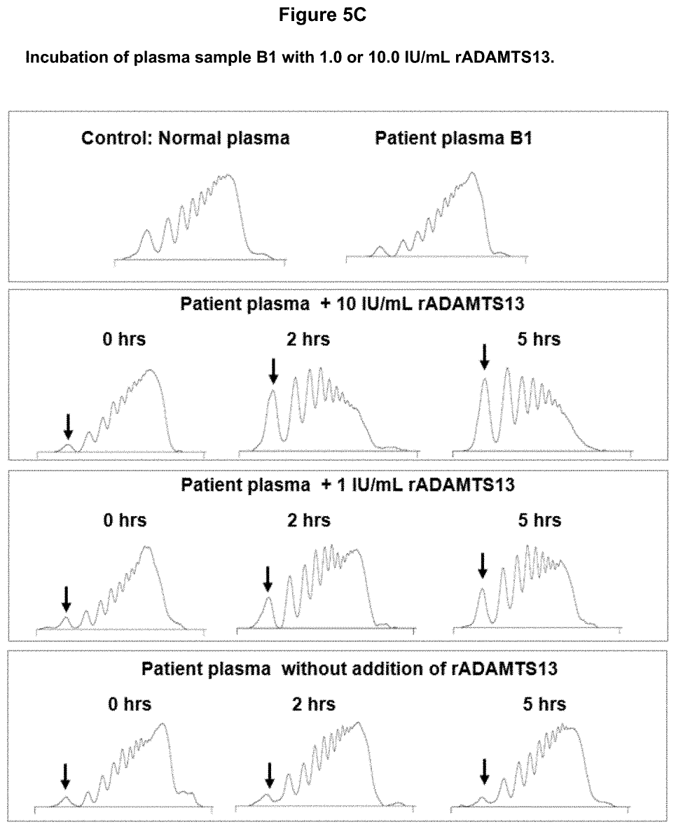

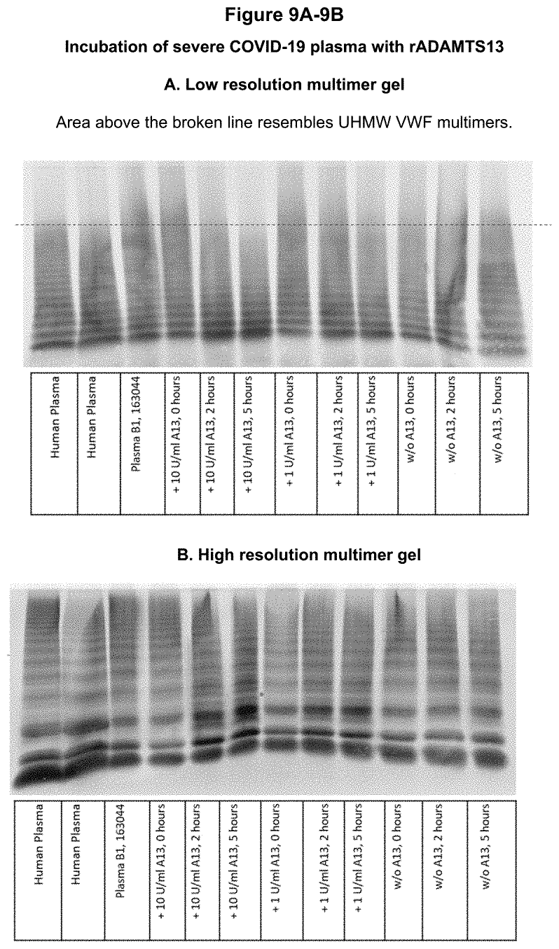

The effect of supplementing ADAMTS13 to higher levels was tested using plasma from a patient with greater VWF/ADAMTS13 mismatch (VWF:Ag 5.76 IU/mL; ADAMTS13 0.43 IU/mL; ratio 13.4). Results from these experiments are shown in FIGS. 5A-5C and FIGS. 9A-9B. Incubation with 10 IU/mL rADAMTS13 resulted in a progressive reduction in VWF activity over time achieving more than a 60% reduction at 5 h (FIG. 5A) and disappearance of all the abnormal UHMW VWF multimers visualized using the semi-automated electrophoresis method (FIG. 5B). The effect of 1 IU/mL rADAMTS13 was sufficient to cause a reduction in VWF activity and a visible depletion of UHMW VWF within 2 h, and within 5 h, VFW activity reduced by about 30%. Disappearance of UHMW VWF was accompanied by marked increase in low molecular weight multimers and dimers, and also by a more pronounced appearance of satellite bands (FIG. 5C). These findings were also present with the home cast gels, which showed that the elevated UHMW multimer quantitation parameter of 124% reduced by 16% after 5 h of incubation with 1 IU/mL rADAMTS13 and by 36% with 10 IU/mL rADAMTS13, to a level lower than that observed in control plasma (88%). Exposure of VWF to rADAMTS13 resulted in the appearance of higher quantities of lower sized multimers with an increase in stainable satellite bands indicative of proteolytic cleavage of VWF. In a related experiment, the patient plasma containing 5.76 IU/mL VWF:Ag was incubated with 1 and 10 IU/mL rADAMTS13 and analyzed for VWF multimer distribution with two different electrophoresis gel systems: low resolution (FIG. 9A) and high resolution (FIG. 9B). Controls were normal human plasma, patient plasma without the addition of any reagents, and patient plasma incubated under identical conditions but without the addition of rADAMTS13. Increasing time of exposure of VWF to rADAMTS13 resulted in a more pronounced proteolysis of VWF visible by the appearance of an increase in proteolytic fragments and the stronger stain of lower molecular weight forms of VWF (FIG. 9B).

In this exploratory study, the VWF/ADAMTS13 axis was evaluated in adults admitted to two hospitals for mechanical ventilation due to severe COVID-19. The inclusion criteria enabled enrollment of a cross-sectional cohort of patients with early stage severe COVID-19, and also those with established disease requiring prolonged ventilation. The cohort contained a preponderance of older males with multiple comorbidities and was over-represented with patients from black and ethnic minority groups and with non-O blood groups. These characteristics, and the frequency of adverse outcomes mirrors previously reported severe COVID-19 cohorts. See, Docherty et al. BMJ 2020; 369:m1985; Petrilli C M et al. BMJ 2020; 369:m1966. The striking finding in this clinically heterogeneous cohort was that circulating levels of VWF were substantially elevated in all of the patients, in some cases, up to ten-fold higher than normal levels. According to the present disclosure, the extent to which VWF is dysregulated in this severe COVID-19 cohort is greater than in any previously reported inflammatory illness. This is in line with observations of others in severe COVID-19 patients and supports the hypothesis that endothelial inflammation following SARS-CoV-2 infection results in dysregulated release of granular stores of VWF into the circulation, particularly UHMW VWF multimers. See, Goshua, supra; Morici, supra; and Escher, supra; Varga, supra; Bernardo et al., Blood 2004; 104:100-6.

Most patients with severe COVID-19 also had decreased circulating levels of ADAMTS13 protein and ADAMTS13 proteolytic activity typically between 30 to 70 percent of normal levels, resulting in an abnormal VWF/ADAMTS13 ratio. These data are in line with observations from COVID-19 case series of reduced ADAMTS13 activity or markedly increased VWF/ADAMTS13 ratio. See, Morici, supra; Blasi supra; Martinelli, supra. In other cohorts of patients, however, significantly reduced ADAMTS13 levels were not seen in COVID-19, but all studies shared a VWF/ADAMTS13 ratio that was significantly higher in COVID-19 than in any of the control groups. See, Escher 192:174, supra; Doevelaar A A N et al. medRxiv 2020. Although in a further small case series in which comprehensive hemostasis testing was performed longitudinally in COVID-19 patients for the duration of hospital admission, reduced ADAMTS13 was an inconsistent finding See, M. Hardy, et al., Thromb. Res. 197 (2020) 20-23. It is noteworthy that low ADAMTS13 plasma levels predicted mortality in COVID-19 patients in three independent studies. See, Tiscia et al., TH Open 4 (3) (2020) e203-e206; Bazzan et al., Internal and Emergency Med. 15 (5) (2020) 861-863; Sweeney, et al., Evidence for Secondary Thrombotic Microangiopathy in COVID-19, MedRxiv, 2020.

One possible explanation for reduced ADAMTS13 levels is sequestration of ADAMTS13 by high levels of circulating high molecular weight VWF multimers through a low affinity interaction that does not result in multimer cleavage. See, Feys et. al., J Thromb Haemost 2009; 7:2088-95, similar to the transient reduction in ADAMTS13 that follows the sudden VWF release after desmopressin treatment. See, Reiter R A et al., Blood 2003; 101:946-8. Normal ADAMTS13 synthesis from cultured human cell lines is inhibited by inflammatory cytokines such as IFN-γ, IL-4 and TNF-α. See, Cao W J et al., J Thromb Haemost 2008; 6:1233-5. Inflammatory cytokines may also inhibit ADAMTS13-mediated cleavage of UHMW VWF multimers. See, Bernardo, supra. This raises the possibility that during the cytokine storm that accompanies severe COVID-19, there may be both reduced synthesis and functional inhibition of ADAMTS13.

These data also show that in all of the COVID-19 patients, the reciprocal changes in the plasma levels of VWF and ADAMTS13 were accompanied by abnormal circulating UHMW VWF multimers that are completely absent in healthy controls. Circulating UHMW VWF multimers have been observed with trauma, sepsis-associated disseminated intravascular coagulation, severe malaria, and after endotoxin infusion in healthy volunteers See Dyer et al., Transfusion. 2020 June; 60(6):1308-131; Kremer et al., J Thromb Haemost 2007; 5:2284-90; Larkin et al., PLoS Pathog 2009; 5: e1000349; Reiter R A et al., Thromb Haemost 2005; 93:554-8, most likely resulting from an excess of the substrate VWF compared to ADAMTS13 resulting in incomplete VWF cleavage. Notably, while nearly half of the patients of the present disclosure had evidence of bacterial infection, particularly localized to intravascular lines, none displayed disseminated intravascular coagulation (DIC) or any other previously recognized cause for VWF/ADAMTS13 mismatch. The extent of the mismatch between was also much greater than reported in other inflammatory settings, resulting in higher levels of thrombogenic UHMW VWF multimers.

This study indicated reduction of ADAMTS13 in all patients with some having levels going down to 20% of the reference level. The study results, therefore, favor the hypothesis that COVID-19 is indeed accompanied by a relevant reduction of ADAMTS13. It remains open if it is a primary or secondary deficiency as very high VWF as substrate could consume its enzyme. However, a correlation was not seen between very high VWF and relatively low ADAMTS13.

These observations directly support the recently proposed hypothesis that coagulopathy following COVID-19 is driven by a potent inflammatory response resulting in disordered regulation of multiple components of hemostasis, including the coagulation and fibrinolysis pathways as well as the platelet-endothelium interaction. See, Morici, supra; Fogarty et al. Br J Haematol 2020; 89:1060-1. Likewise, findings disclosed herein specifically implicate abnormal release of granular stores of VWF, and dysregulated proteolysis of VWF in the pathogenesis of thrombosis. See, Varga, Lancet 395 (10234) (2020) 1417-1418; Bernardo et al. supra; R. Escher 190:62, supra; Goshua, supra; Morici, supra.

The present investigation is the first to directly visualize that a consequence of VWF/ADAMTS13 mismatch is the presence of UHMW VWF multimers in COVID-19 plasma samples. The UHMW VWF multimers levels in some COVID-19 patients were approaching those of the acute TTP control. However, even though micro-vascular occlusion is a feature of both COVID-19 and acute TTP, the COVID-19 coagulopathy is distinct because of the absence of TTP features such as severe thrombocytopenia, circulating red cell schistocytes and severe reduction in ADAMTS13 level. Therefore, findings of the present disclosure fit best with a model in which VWF dysregulation is an important component of COVID-19 coagulopathy but that dysfunction of other hemostatic pathways are contributory. See, T. Iba et al., Inflamm Res 69 (12) (2020) 1181-1189.

Observations suggest that circulating UHMW VWF multimers arising from dysregulated VWF/ADAMTS13 is likely to be a significant component of this coagulopathy that accounts for some of the clinical findings in severe COVID-19. Although anticoagulation was associated with lower mortality and intubation among hospitalized COVID-19 patients, it is significant that anti-thrombotic drugs such as heparin that target the coagulation pathway may be less effective in preventing thrombosis in severe COVID-19 than in other inflammatory diseases. See, Nadkarni G N, et al., J Am Coll Cardiol. 2020 Aug. 26. E-published DOI: 10.1016/j.jacc.2020.08.041; Cattaneo M, Bertinato E M, Birocchi S, et al. Pulmonary Embolism or Pulmonary Thrombosis in COVID-19? Is the Recommendation to Use High-Dose Heparin for Thromboprophylaxis Justified? Thromb Haemost 2020.

The study results obtained by in vitro incubation of severe COVID-19 plasma with rADAMTS13 clearly shows that rising ADAMTS13 levels reduces VWF activity and, most importantly, proteolytically degrades UHMW VWF multimers. UHMW VWF is supposed to be the stickiest form of VWF which triggers formation of platelet thrombi observed in COVID-19 patients similar to TTP. See, Albiol N et al., Ann Hematol. 2020; 99(7):1673-1674; Capecchi M et al., Haematologica 105(10) 2020; haematol.2020.262345; Doevelaar A A N, supra. The VWF-ADAMTS13 axis is tractable to interventions such as ADAMTS13 replacement. Scully M et al., Blood 2017; 130:2055-63. It should now be considered as an additional therapeutic target alongside the coagulation pathway. rADAMTS in several in vitro studies, in animal models, and a clinical study has demonstrated its efficacy to cleave human VWF and degrading UHMW VWF multimers, thus, restoring the dysregulated protein pattern seen in TTP and similar pathologies. See, Plaimauer B et al., J Thromb Haemost. 2011; 9(5):936-944; Crescente M et al., Thromb Haemost. 2012; 108(3):527-532; Kopić A et al., J Thromb Haemost. 2016; 14(7):1410-1419.

Classical anticoagulation therapy in COVID-19 patients is unlikely an ideal treatment for micro-thrombosis in arterioles of the lungs and other organs. The data in this Example demonstrates the usefulness of rADAMTS in COVID-19 patients, as rADAMTS13 treatment better aims to restore the balance between VWF and ADAMTS13 by increasing ADAMTS13 activity, which in turn reduces up-regulated VWF activity.

Findings disclosed herein are significant for the management of patients with COVID-19 because the VWF-ADAMTS13 axis is a therapeutically accessible target. Data of this Example demonstrates early evidence of the utility of ADAMTS13 replacement by showing that incubation of patient samples with rADAMTS13 resulted in a time and concentration dependent reduction in VWF activity, even in a sample with the most severe mismatch between VWF and ADAMTS13. Further it is shown that this correction of abnormally high VWF activity was accompanied by the disappearance of UHMW VWF multimers and a time-dependent increase in lower molecular weight multimers with more intense satellite bands indicating ex vivo proteolysis. This pharmacodynamic effect of ADAMTS13 replacement mirrors that observed in other coagulopathies in which rADAMTS13 has been evaluated in early phase clinical trials including in patients with VWF/ADAMTS13 mismatch because of severe congenital TTP and in plasma samples from patients with autoimmune TTP. See, Scully et al., Blood 130 (19) (2017) 2055-2063; Plaimauer et al., J. Thromb. Haemost. 9 (5) (2011) 936-944. rADAMTS13 also results in dissolution of thrombus in flow chamber models and in a small animal model of VWF/ADAMTS13 mismatch. See, Haemost et al., supra.; Shenkman, et. al, Thromb Haemost 96 (2) (2006) 160-166; Crescente et al., Thromb. Haemost. 108 (3) (2012) 527-532. In the present study, ex vivo proteolytic activity in the COVID-19 plasma samples was even observed after correction of ADAMTS13 levels to 1.0 to 1.5 IU/mL, which was in the range achieved in the phase 1 congenital TTP study following infusion of 40 IU/Kg rADAMTS13 which was well tolerated and was not associated with significant adverse events. See, Scully, supra. However, it is also noteworthy that the ADAMTS13 activity required to cleave abnormal UHMW VWF multimers in static models may be lower than in conditions of flow. See, Dong, J. Whitelock, A. Bernardo et al., supra.

Observations herein suggest that circulating UHMW VWF multimers arising from dysregulated VWF/ADAMTS13 is likely to be a significant component of this coagulopathy that accounts for some of the clinical findings in severe COVID-19. Although anticoagulation was associated with lower mortality among hospitalized COVID-19 patients it is significant that anti-thrombotic drugs such as heparin that target the coagulation pathway may be less effective in preventing thrombosis in severe COVID-19 than in other inflammatory diseases. See, G. N. Nadkarni et al., J Am Coll Cardiol 76 (16) (2020) 1815-1826; M. Cattaneo et al., Thromb. Haemost. 120 (8) (2020) 1230-1232. The dramatic abnormalities in the VWF-ADAMTS13 axis demonstrated in this Example provides a potential explanation for this. The demonstration that ADAMTS13 replacement restores VWF homeostasis in COVID-19 plasma samples suggests that a purified rADAMTS13 should be considered as a potential therapeutic intervention for COVID-19 coagulopathy alongside other anti-thrombotic therapies.

### Example 10—VWF, ADAMTS13, and Other Assays

The following materials and methods were used in connection with Example 9.

Determination of VWF antigen (VWF:Ag). VWF:Ag was determined using the Asserachrom VWF:Ag test kit (Diagnostica Stago, Asnieres sur Seine, France). Patient plasma was tested at eight different dilutions. VWF was captured by rabbit anti-human F(ab′)2 fragments coated on the internal walls of a microplate well. Subsequent to a washing step, bound VWF was detected by adding polyclonal anti-human VWF antibodies coupled with peroxidase. Peroxidase activity was detected by using tetramethyl-benzidine (TMB) as substrate. The absorbance was read at 450 nm using a microplate reader. VWF:Ag was calculated relative to the assay reference standard (human normal plasma).

Determination of VWF ristocetin cofactor activity (VWF:RCo). VWF:RCo activity was measured using the BCS coagulation system analyzer (Behring Coagulation System, BCS, Siemens, Germany). VWF of the patient plasma causes agglutination of stabilized platelets in the presence of ristocetin, both contained in the “von Willebrand reagent” (Behring Coagulation System, BCS, Siemens, Germany). The agglutination reduces the turbidity of the reagent preparation, and the change in optical density is measured by the instrument. The VWF:RCo activity was calculated from a reference curve constructed by different dilutions of a reference plasma, calibrated against the WHO standard.

Determination of VWF collagen binding (VWF:CBA). VWF:CBA was determined using the TECHNOCHROM VWF:CBA test kit (Technoclone, Vienna, Austria). Patient plasma was tested in multiple dilutions. VWF binds to human collagen type III which is coated on the internal walls of a microplate well. Subsequent to a washing step, bound VWF was detected by adding polyclonal anti-human VWF antibodies coupled with peroxidase. Peroxidase activity was detected by using tetramethyl-benzidine (TMB) as substrate. The absorbance was read at 450 nm using a microplate reader. Alternatively, VWF:CBA was determined by the Zymutest test kit (HYPHEN BioMed SAS, Neuville sur Oise, France). VWF:CBA was calculated to the assay calibrators traceable to the WHO standard.

ADAMTS13 assays. Three complementary assays were used to determine activity of ADAMTS13 all obtained from Technoclone (Vienna, Austria). TECHNOZYM ADAMTS13 Activity ELISA is a chromogenic assay which uses GST-VWF73 which is cleaved by ADAMTS13. A specific monoclonal antibody recognizing the cleaved epitope, which is HRP labelled binds to the cleaved peptide. See, Kato S, Matsumoto M, Matsuyama T, Isonishi A, Hiura H, Fujimura Y. Novel monoclonal antibody-based enzyme immunoassay for determining plasma levels of ADAMTS13 activity. Transfusion 2006; 46:1444-52; Kokame K, Matsumoto M, Fujimura Y, Miyata T. VWF73, a region from D1596 to R1668 of von Willebrand factor, provides a minimal substrate for ADAMTS13. Blood 2004; 103:607-12. TECHNOFLOUR ADAMTS13 Activity is an automated assay for the Ceveron s100 instrument. A FRET substrate based on the VWF73 peptide is cleaved by the sample's derived ADAMTS13, resulting in a fluorescent signal. The emitted signal is proportional to the sample's ADAMTS13 activity levels. Ee, Kokame K, Nobe Y, Kokubo Y, Okayama A, Miyata T. FRETS-VWF73, a first fluorogenic substrate for ADAMTS13 assay. Br J Haematol 2005; 129:93-100. TECHNOZYM ADAMTS13 Antigen ELISA is a sandwich ELISA employing anti-ADAMTS13 antigen directed to the CUB domain and HRP labelled anti ADAMTS13 polyclonal antibodies. See, Rieger M, Ferrari S, Kremer Hovinga J A, et al. Relation between ADAMTS13 activity and ADAMTS13 antigen levels in healthy donors and patients with thrombotic microangiopathies (TMA). Thromb Haemost 2006; 95:212-20. All ADAMTS13 parameters were calculated using assay specific reference plasma calibrated against the WHO standard. Normal reference ranges for these assays as indicated in Table 3 were established by testing more than 100 pre-COVID samples from healthy blood donors.

Discrepancy between SARS-CoV-2 PCR and ELISA. From the 36 patients included in the study, 6 patients had no confirmed positive PCR from the nasopharyngeal swabs. Among the various ways to perform RT-PCR, pharyngeal or nasal swabs have been shown to only exhibit a moderate sensitivity despite their very high specificity. For most accurate RT-PCR diagnostics, a bronchioalveolar lavage would be suitable, but is unrealistic to be performed on every patient. Thus, one patient negative for SARS-Cov-2 RNA exhibited high titre antibodies against the SARS-CoV-2 NP or RBD protein, clearly supporting its COVID-19 diagnose. Five residual patients, who were SARS-CoV-2 PCR negative and anti-SARS-CoV-2 IgG negative, could still be COVID-19 patients. Depending on the time after initial infection to development of an IgG mediated immune response it could vary from patient to patient and is dependent on the general constitution of the patient likewise to the patient's medication regime. For the immunological assay's positive agreement with RT-PCR varied from 6-15% in patients less than 5 days after reported infection, to 34-46% between 5-10 days and 100% in patients more than 15 days after initial infection.

Abbreviations

ADAMTS13 A disintegrin and metalloprotease with a thrombospondin type 1 motif member 13
COVID-19 Coronavirus disease 2019
ELISA Enzyme-linked immunosorbent assay
FRETS Fluorescent resonance energy transfer
PCR Polymerase chain reaction
SARS-CoV-2 Severe acute respiratory syndrome coronavirus 2
TTP Thrombotic thrombocytopenia purpura
UHMW Ultra-high molecular weight
VWF von Willebrand factor
VWF:Ag von Willebrand factor antigen
VWF:CB von Willebrand factor collagen binding activity
VWF:RCo von Willebrand factor ristocetin cofactor activity
ADAMTS13:Ag ADAMTS13 antigen
ADAMTS13:ELISA ADAMTS13 activity measured by ELISA
ADAMTS13:FRETS ADAMTS13 activity measured by FRETS

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate and are provided for description.

Patents, patent applications, publications, product descriptions, and protocols are cited throughout this application, the disclosures of which are incorporated herein by reference in their entireties for all purposes.

What is claimed is:

1. A method of treating or preventing at least one condition or complication in a subject infected with severe acute respiratory syndrome coronavirus 2 (SARS-CoV-2) or suffering from coronavirus disease 2019 (COVID-19), the method comprising administering to the subject in need thereof a therapeutically effective amount of a composition comprising A disintegrin and metalloproteinase with a thrombospondin type 1 motif (ADAMTS13).

2. A method of treating a subject at risk of developing at least one condition or complication associated with severe acute respiratory syndrome coronavirus 2 (SARS-CoV-2) infection or coronavirus disease 2019 (COVID-19), the method comprising administering to the subject in need thereof a therapeutically effective amount of a composition comprising A disintegrin and metalloproteinase with a thrombospondin type 1 motif (ADAMTS13).

3. A method of treating or preventing at least one condition or complication in a subject infected with severe acute respiratory syndrome coronavirus 2 (SARS-CoV-2) or suffering from coronavirus disease 2019 (COVID-19), comprising the steps of:
a) administering to the subject in need thereof a therapeutically effective amount of a pharmaceutical composition comprising a recombinant A Disintegrin And Metalloproteinase with Thrombospondin type 1 motif, member-13 (ADAMTS13), wherein said therapeutically effective amount is sufficient to:
i) reduce circulating ultra-high molecular weight (UHMW) von Willebrand factor (VWF) multimers to a level that is at least about 5%, at least about 10%, or at least about 20% decreased compared to a measured level of VWF in the subject's blood prior to administration;
ii) reduce circulating UHMW VWF multimers to a level that is no more than about 5%, no more than about 10%, or no more than about 20% above a normal VWF baseline value;
iii) reduce circulating VWF to a level that is at least about 5%, at least about 10%, or at least about 20% decreased compared to a measured level of VWF in the subject's blood prior to administration;
iv) reduce circulating VWF to a level that is no more than about 5%, no more than about 10%, or no more than about 20% above a normal VWF baseline value;
v) reduce VWF activity level to a level that is at least about 5%, at least about 10%, or at least about 20% decreased compared to a measured level of VWF activity in the subject's blood prior to administration;
vi) reduce VWF activity level to a level that is no more than about 5%, no more than about 10%, or no more than about 20% above a normal VWF activity baseline value;
vii) increase circulating ADAMTS13 levels from about 100% to about 150% above a normal ADAMTS13 baseline value; or
viii) combinations of i)-vii); and
b) periodically monitoring and adjusting the administered amount to maintain said reduced level of circulating VWF, UHMW VWF multimers, or combinations thereof.

4. The method of any one of claims 1-3, wherein the subject is administered the composition comprising ADAMTS13 before the condition or complication is present.

5. The method of any one of claims 1-4, wherein the subject is administered the composition comprising ADAMTS13 after the condition or complication is present.

6. The method of any one of claims 1-5, wherein the condition or complication is a coagulopathy, blood-clotting disorder, infarction, thrombosis, embolism, stroke, veno-occlusive disorders, prothrombotic conditions, sepsis, renal failure, respiratory failure, acute respiratory distress syndrome (ARDS), chronic obstructive pulmonary disease (COPD), thrombotic microangiopathy (TMA), pneumonia, asthma, hypertension, elevated plasma levels of VWF and/or its multimers (especially ultralarge multimers (UHMW)), elevated plasma VWF activity levels, reduced plasma levels of endogenous ADAMTS13, inflammation, elevated cytokine levels, or combination thereof.

7. The method of claim 6, wherein the thrombosis is deep vein thrombosis (DVT).

8. The method of claim 6, wherein the embolism is a pulmonary embolism (PE).

9. The method of claim 6, wherein the complication is elevated plasma levels of VWF, elevated plasma levels of UHMW VWF multimers, and/or reduced plasma levels of endogenous ADAMTS13.

10. The method of any one of claims 1-9, wherein the subject is 65 years of age or older.

11. The method of any one of claims 1-10, wherein the subject presents with a risk factor.

12. The method of claim 11, wherein the risk factor is elevated plasma levels of VWF, elevated plasma levels of ultralarge multimers (UHMW) VWF multimers, elevated plasma VWF activity levels, reduced plasma levels of endogenous ADAMTS13, reduced activity of endogenous ADAMTS13 activity, elevated cytokine levels, coagulopathies, blood-clotting disorders, veno-occlusive disorders, prothrombotic conditions, inherited thrombotic thrombocytopenic purpura (TTP), acquired TTP, disseminated intravascular coagulation (DIC), sepsis, sickle cell, respiratory failure, acute respiratory distress syndrome (ARDS), chronic obstructive pulmonary disease (COPD), thrombotic microangiopathy (TMA), pneumonia, asthma, pregnancy, menopause, pen-menopause, hypertension, pulmonary hypertension, thromboses, embolism, myocardial infarction, stroke, cough, shortness of breath, pulmonary infiltrates, respiratory failure, elevated plasma fibrogen, activated hemostasis pathway, intensive care unit (ICU) admission, or combinations thereof.

13. The method of claim 12, wherein the risk factor is a elevated plasma levels of VWF, elevated plasma levels of UHMW VWF multimers, and/or reduced plasma level of endogenous ADAMTS13.

14. The method of claim 12, wherein the risk factor is a prothrombotic condition.

15. The method of any one of claims 1-14, wherein administering the composition comprising ADAMTS13 to the subject reduces the duration, severity, or frequency of occurrence of the condition or complication compared to a subject that was not administered the composition comprising ADAMTS13; b) reduces plasma level of VWF protein, plasma level of VWF multimers, VWF activity, plasma ratio of VWF to ADAMTS13 (VWF:A13), platelet aggregation, blood clotting, thrombosis, embolism, infarction, veno-occlusion, stroke, inflammation, plasma cytokine levels, or combinations thereof as compared to a normal baseline range in a healthy individual; c) reduces plasma level of VWF protein, plasma level of VWF multimers, VWF activity, plasma VWF:A13, or combinations thereof; d) reduces platelet aggregation, blood clotting, thrombosis, embolism, infarction, veno-occlusion, stroke, or combinations thereof; e) increases plasma levels of ADAMTS13, plasma ADAMTS13 activity, or combinations thereof to a normal baseline range in a healthy individual; or f) a combination of a)-e).

16. The method of claim 15, wherein the VWF multimer is an UHMW multimer.

17. The method of any one of claims 1-16, wherein administering the composition comprising ADAMTS13 to the subject increases plasma level of ADAMTS13, plasma ADAMTS13 activity, or combinations thereof from about 20-100%, above a normal baseline range or normal baseline value of ADAMTS13 plasma level or ADAMTS13 activity level.

18. The method of any one of claims 1-17, wherein administering the composition comprising ADAMTS13 to the subject increases plasma level of ADAMTS13, plasma ADAMTS13 activity, or combinations thereof from about 100-150%, compared to a normal baseline value of ADAMTS13 plasma level or ADAMTS13 activity level.

19. The method of any one of claims 1-18, wherein the therapeutically effective amount of the ADAMTS13 is about 10-400 IU/kg for a subject having a VWF level that is more than about 5% higher than a baseline corresponding to the upper limit of a predetermined normal range of VWF levels in healthy subjects.

20. The method of any one of claims 1-19, wherein the therapeutically effective amount of the ADAMTS13 is about 10-400 IU/kg or about 40-400 IU/kg for a subject having a VWF level that is at least about two times higher than a normal baseline VWF level in healthy subjects.

21. The method of any one of claims 1-18, wherein the therapeutically effective amount of the ADAMTS13 is about 10-400 IU/kg or about 40-400 IU/kg for a subject having a VWF level that is at least about three times higher than a normal baseline VWF level in healthy subjects.

22. The method of any one of claims 1-18, wherein the therapeutically effective amount of the ADAMTS13 is about 10-400 IU/kg for a subject having an ADAMTS13 activity and/or level between about 30-70% that of a normal ADAMTS13 baseline activity and/or level in healthy subjects.

23. The method of any one of claims 1-18, wherein the therapeutically effective amount of the ADAMTS13 is about 10-400 IU/kg or about 40-400 IU/kg for a subject having an ADAMTS13 activity and/or level less than about 20% of a normal ADAMTS13 baseline activity and/or level in healthy subjects.

24. The method of any one of claims 1-18, wherein the therapeutically effective amount of the ADAMTS13 is about 10-400 IU/kg or about 40-400 IU/kg for a subject having a ultra-high molecular weight (UHMW) VWF multimer level between about 100-130% that of a normal UHMW VWF multimer baseline level in healthy subjects.

25. The method of any one of claims 1-18, wherein the therapeutically effective amount of the ADAMTS13 is about 10-400 IU/kg for a subject having a ultra-high molecular weight (UHMW) VWF multimer level at least about 101%, at least about 105%, or at least about 107% that of a normal UHMW VWF multimer baseline level in healthy subjects.

26. The method of any one of claims 1-25, wherein the therapeutically effective amount of the ADAMTS13 is about 10-320 IU/kg, about 10-300 IU/kg, about 10-200 IU/kg, about 10-180 IU/kg, about 10-160 IU/kg, about 10-80 IU/kg, about 10-60 IU/kg, about 10-40 IU/kg, about 10-20 IU/kg, about 20-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, or about 20-30 IU/kg, about 30-320 IU/kg, about 30-300 IU/kg, about 30-180 IU/kg, about 30-160 IU/kg, about 30-60 IU/kg, about 40-400 IU/kg, about 40-320 IU/kg, about 40-300 IU/kg, about 40-180 IU/kg, about 40-160 IU/kg, about 40-80 IU/kg or about 40-60 IU/kg.

27. The method of any one of claims 1-25, wherein the therapeutically effective amount of the ADAMTS13 is about 10-40 IU/kg, about 10-20 IU/kg, about 40-320 IU/kg, about 40-160 IU/kg, about 40-80 IU/kg, or about 40-60 IU/kg.

28. The method of any one of claims 1-27, wherein the composition comprising ADAMTS13 is administered monthly, every two weeks, weekly, twice a week, three times a week, every other day, daily, every 12 hours, every 8 hours, every six hours, every four hours, every two hours, or every hour.

29. The method of any one of claims 1-27, wherein the composition comprising ADAMTS13 is administered intravenously, subcutaneously, via intravenous bolus, or via intravenous infusion.

30. The method of any one of claims 1-29, wherein the composition comprising ADAMTS13 comprises plasma derived human ADAMTS13.

31. The method of any one of claims 1-29, wherein the composition comprising ADAMTS13 comprises recombinant ADAMTS13.

32. The method of any one of claims 1-31, comprising the further steps of:
periodically measuring the subject's VWF level; and
reducing the therapeutically effective amount to about 10-100 IU/kg when the subject's VWF level is within a predetermined baseline range of a healthy individual.

33. The method of any one of claims 15-32, wherein a normal baseline range for VWF levels is a range of about 50-200% or about 42-136% of an established or predetermined average baseline.

34. The method of any one of claims 15-33, wherein a normal baseline range for ADAMTS13 levels is a range of about 40-160% or about 87-113% of an established or predetermined average baseline.

35. A method of determining whether a subject diagnosed with COVID-19 is at an increased risk for a thrombotic coagulopathy, said method comprising the steps of:
a) measuring in a blood plasma sample one or more of:
i) a plasma level of VWF protein;
ii) an activity level of VWF in the plasma sample;
iii) a plasma level of UHMW VWF protein multimers;
iv) a plasma level of ADAMTS13 protein; or
v) an activity level of ADAMTS13 protein in the plasma sample; and
b) comparing the plasma level(s) or activity level(s) measured in step a) to a baseline range or baseline value for the same plasma level(s) or activity level(s); and
c) identifying the subject being at risk for a thrombotic coagulopathy when at least one of the following is met:
i) the plasma level of VWF protein is increased;
ii) the activity level of VWF is increased;
iii) plasma UHMW VWF protein multimers are detected or the plasma level of UHMW VWF protein multimers is increased;
iv) the plasma level of ADAMTS13 protein is decreased; or
v) the activity level of ADAMTS13 protein is decreased,
as compared to the baseline range or baseline value for the same plasma level(s) or activity level(s).

36. The method of claim 35, wherein thrombotic coagulopathy includes platelet aggregation, blood clotting, a thrombosis, a thrombotic microangiopathy, an embolism, an infarction, veno-occlusion, a stroke, renal failure resulting from thrombosis, or combinations thereof.

37. The method claim 35 or claim 36, wherein the subject is at risk for developing a thrombotic coagulopathy when the plasma level of VWF protein is about 120% to about 300% of the baseline value for said VWF protein plasma level.

38. The method of any one of claims 35-37, wherein the subject is at risk for developing a thrombotic coagulopathy when the plasma level of VWF protein is about 300% or more of the baseline value for said VWF protein plasma level.

39. The method of any one of claims 35-38, wherein the subject is at risk for developing a thrombotic coagulopathy when the activity level of VWF in the plasma sample is about 120% to about 300% of the baseline value for said VWF activity level.

40. The method of any one of claims 35-39, wherein the subject is at risk for developing a thrombotic coagulopathy when the activity level of VWF in the plasma sample is about 300% or more of the baseline value for said VWF activity level.

41. The method of any one of claims 35-40, wherein the subject is at risk for developing a thrombotic coagulopathy when the plasma level of ADAMTS13 protein is about 70% to about 100% of the baseline value for said ADAMTS13 protein plasma level.

42. The method of any one of claims 35-41, wherein the subject is at risk for developing a thrombotic coagulopathy when the plasma level of ADAMTS13 protein is 70% or less of the baseline value for said ADAMTS13 protein plasma level.

43. The method of any one of claims 35-42, wherein the subject is at risk for developing a thrombotic coagulopathy when the activity level of ADAMTS13 in the plasma sample is about 70% to about 100% of the baseline value for said ADAMTS13 activity level.

44. The method of any one of claims 35-43, wherein the subject is at risk for developing a thrombotic coagulopathy when the activity level of ADAMTS13 in the plasma sample is 70% or less of the baseline value for said ADAMTS13 activity level.

45. The method of any one of claims 35-44, wherein the subject is at risk for developing a thrombotic coagulopathy when the plasma level of UHMW VWF multimers is about 100% to about 110% of the baseline value for said UHMW VWF multimer plasma level.

46. The method of any one of claims 35-45, wherein the subject is at risk for developing a thrombotic coagulopathy when the plasma level of UHMW VWF multimers is 110% or more of the baseline value for said UHMW VWF multimer plasma level.

47. The method of any one of claims 35-46, wherein the subject is at risk for developing a thrombotic coagulopathy when the ratio of VWF:A13 levels in the plasma sample is 3 or less.

48. The method of any one of claims 35-47, wherein the subject is at risk for developing a thrombotic coagulopathy when the ratio of VWF:A13 levels in the plasma sample is greater than 3.

49. The method of any one of claims 35-48, wherein the baseline value is a predetermine value based on a normal control population.

50. The method any one of claims 35-49, wherein the baseline value is a mean of a predetermine range of a normal control population.

51. A method of determining whether a subject diagnosed with COVID-19 is at risk for a thrombotic coagulopathy, said method comprising the steps of:
a) measuring in a blood plasma sample one or more of:
i) a plasma level of VWF protein;
ii) an activity level of VWF in the plasma sample;
iii) a plasma level of UHMW VWF protein multimers;
iv) a plasma level of ADAMTS13 protein; or
v) an activity level of ADAMTS13 protein in the plasma sample; and
b) identifying the subject being at risk for a thrombotic coagulopathy when at least one of the following is met:
i) the plasma level of VWF protein is at least about 1.2 IU/ml;
ii) the VWF activity level is at least about 1.2 IU/ml or 1.8 IU/ml;
iii) plasma UHMW VWF protein multimers are detected;
iv) the plasma level of ADAMTS13 protein no more than about 0.7 IU/ml; or
v) the activity level of ADAMTS13 protein is no more than about 0.8 or about 0.9 IU/ml.

52. The method of claim 51, wherein in step b) the subject is at a high risk for a thrombotic coagulopathy when at least one of the following is met:
i) the plasma level of VWF protein is at least about 4.5 IU/ml;
ii) the VWF activity level is at least about 3.3 IU/ml or 4.4 IU/ml;
iii) the plasma level of ADAMTS13 protein no more than about 0.4 IU/ml; or
iv) the activity level of ADAMTS13 protein is no more than about 0.4 or about 0.5 IU/ml.

53. The method of any one of claims 35-52, wherein the method further comprises administering to the subject a composition comprising a therapeutically effective amount of ADAMTS13.

54. The method of claim 53, wherein the therapeutically effective amount of the ADAMTS13 is about 10-400 IU/kg, about 10-320 IU/kg, about 10-300 IU/kg, about 10-200 IU/kg, about 10-180 IU/kg, about 10-160 IU/kg, about 10-80 IU/kg, about 10-60 IU/kg, about 10-40 IU/kg, about 10-20 IU/kg, about 20-320 IU/kg, about 20-300 IU/kg, about 20-200 IU/kg, about 20-180 IU/kg, about 20-160 IU/kg, about 20-80 IU/kg, about 20-60 IU/kg, about 20-40 IU/kg, about 20-30 IU/kg, about 30-320 IU/kg, about 30-300 IU/kg, about 30-180 IU/kg, about 30-160 IU/kg, about 30-60 IU/kg, about 40-400 IU/kg, about 40-320 IU/kg, about 40-300 IU/kg, about 40-180 IU/kg, about 40-160 IU/kg, about 40-80 IU/kg or about 40-60 IU/kg.

55. The method of any one of claim 37, 39, 41, 43, 45, 47, or 51, wherein the method further comprises administering to the subject a composition comprising a therapeutically effective amount of ADAMTS13 and wherein the therapeutically effective amount of the ADAMTS13 is about 10-40 IU/kg, about 10-30 IU/kg, about 10-20 IU/kg, about 20-40 IU/kg, or about 20-30 IU/kg.

56. The method of claim 55, wherein the therapeutically effective amount of the ADAMTS13 is about 10 IU/kg, about 20 IU/kg, about 30 IU/kg, or about 40 IU/kg.

57. The method of any one of claim 38, 40, 42, 44, 46, 48, or 52, wherein the method further comprises administering to the subject a composition comprising a therapeutically effective amount of ADAMTS13 and wherein the therapeutically effective amount of the ADAMTS13 is about 40-400 IU/kg, about 40-320 IU/kg, about 40-300 IU/kg, about 40-180 IU/kg, about 40-160 IU/kg, about 40-80 IU/kg or about 40-60 IU/kg.

58. The method of claim 57, wherein the therapeutically effective amount of the ADAMTS13 is about 40 IU/kg, about 60 IU/kg, about 80 IU/kg, or about 160 IU/kg.

59. A kit for determining whether a subject diagnosed with COVID-19 is at risk for a thrombotic coagulopathy, said kit comprising (i) one or more reagents for determining one or more of the plasma level of VWF protein, activity level of VWF, plasma level of UHMW VWF multimers, plasma level of ADAMTS13 protein, activity level of ADAMTS13, (ii) optionally packaging and/or instructions for use, and (iii) optionally one or more reagents for detecting SARS-CoV-2 or diagnosing COVID-19.
